(19) United States
(12) Patent Application Publication
(10) Pub. No.: US 2026/0263518 A1
(43) Pub. Date: Sep. 10, 2026

(54) ENGINEERED IMMUNE CELLS EXPRESSING ANTI-U5 SNRNP200 ANTIBODIES AND USES THEREOF

(71) Applicants: Memorial Sloan-Kettering Cancer Center, New York, NY (US); Memorial Hospital for Cancer and Allied Diseases, New York, NY (US); Sloan-Kettering Institute for Cancer Research, New York, NY (US)

(72) Inventors: Omar Abdel-Wahab, New York, NY (US); Anthony Daniyan, New York, NY (US); David Knorr, New York, NY (US); Katherine Knorr, New York, NY (US); Takeshi Fujino, New York, NY (US)

(21) Appl. No.: 19/165,981

(22) PCT Filed: Mar. 15, 2024
(86) PCT No.: PCT/US2024/020184

§ 371 Date: Sep. 16, 2025

Related U.S. Application Data
(60) Provisional application No. 63/452,981, filed on Mar. 17, 2023.

(51) Int. Cl. A61K 35/17; A61K 38/00; A61K 40/11; A61K 40/31; A61K 40/32; A61K 40/42; A61K 40/50; A61P 35/04; C07K 14/54; C07K 14/705; C07K 16/40; C12N 5/078; C12N 15/867

(52) U.S. Cl. CPC A61K 35/17; A61K 38/00; A61K 40/11; A61K 40/31; A61K 40/32; A61K 40/42; A61K 40/50; A61P 35/04; C07K 14/54; C07K 14/7051; C07K 16/40; C12N 5/0634; C12N 15/867; A61K 2239/48; C07K 2317/54; C07K 2317/55; C07K 2317/565; C07K 2317/622; C07K 2319/03; C12N 2510/00; C12N 2830/34

(57) ABSTRACT

Provided herein are compositions, kits, and methods for manufacturing cells for adoptive cell therapy comprising engineered immune cells that express express a U5 snRNP200-specific receptor (e.g., a U5 snRNP200-specific chimeric antigen receptor), and uses thereof.

### CROSS-REFERENCE TO RELATED APPLICATIONS

This application is the U.S. National Phase application of International Application No. PCT/US2024/020184, filed Mar. 15, 2024, which claims the benefit of and priority to U.S. Provisional Application No. 63/452,981, filed Mar. 17, 2023, the contents of which are incorporated herein by reference in its entirety.

### SEQUENCE LISTING

The contents of the .xml file submitted electronically herewith are incorporated herein by reference in their entirety, filename: 115872-3324_SL.xml; Size: 90,112 bytes; and Date of Creation: Sep. 4, 2025.

### TECHNICAL FIELD

The present technology relates generally to compositions including engineered immune cells that express a U5 snRNP200-specific receptor (e.g., a U5 snRNP200-specific chimeric antigen receptor), and uses thereof.

### BACKGROUND

The following description of the background of the present technology is provided simply as an aid in understanding the present technology and is not admitted to describe or constitute prior art to the present technology.

Despite recent advances in the treatment of acute myeloid leukemia (AML), the 5-year survival for most adult patients is less than 10%. Furthermore, therapeutic options are limited for those unfit to receive cytotoxic chemotherapy due to age or co-morbidities. One major obstacle in improving outcomes for AML patients has been the lack of successful immunotherapies for the majority of patients with this disease. This challenge is in part due to limited knowledge of target antigens which effectively discriminate malignant cells from vital normal hematopoietic precursor cells.

Accordingly, there is an urgent need for methods and compositions that effectively treat AML.

### SUMMARY OF THE PRESENT TECHNOLOGY

In one aspect, the present disclosure provides an engineered immune cell including a receptor that comprises a U5 snRNP200 antigen binding fragment comprising: a VHCDR1 sequence, a VHCDR2 sequence, and a VHCDR3 sequence of GYYWS (SEQ ID NO: 17), EINHSGSTNYNPSLKS (SEQ ID NO: 18), and GRSTSPLDYYYYYMDV (SEQ ID NO: 19); or GYYWS (SEQ ID NO: 23), EINHSGSTNYNPSLKS (SEQ ID NO: 24), and GPRGMYSSSSGDY (SEQ ID NO: 25); or TYGMH (SEQ ID NO: 29), VIWYDGSNTYYADSVKG (SEQ ID NO: 30), and ARGRGYSAQGNRNRAYYFDY (SEQ ID NO: 31) respectively; and/or a V_{L}CDR1 sequence, a V_{L}CDR2 sequence, and a V_{L}CDR3 sequence of: QGDFLRSYYAS (SEQ ID NO: 20), GKNKRPS (SEQ ID NO: 21), and NSRDRSGNHLV (SEQ ID NO: 22); or RASQGIRNDLG (SEQ ID NO: 26), AAVSLQS (SEQ ID NO: 27), and LQHNSYPRT (SEQ ID NO: 28); or RASQSVSSNLA (SEQ ID NO: 32), GAFTRVT (SEQ ID NO: 33), and QQYNDRPPYT (SEQ ID NO: 34), respectively, and/or a nucleic acid encoding the receptor. In some embodiments, the U5 snRNP200 antigen binding fragment comprises (A) a VHCDR1 sequence, a VHCDR2 sequence, and a VHCDR3 sequence of GYYWS (SEQ ID NO: 17), EINHSGSTNYNPSLKS (SEQ ID NO: 18), and GRSTSPLDYYYYYMDV (SEQ ID NO: 19), respectively; and a V_{L}CDR1 sequence, a V_{L}CDR2 sequence, and a V_{L}CDR3 sequence of: QGDFLRSYYAS (SEQ ID NO: 20), GKNKRPS (SEQ ID NO: 21), and NSRDRSGNHLV (SEQ ID NO: 22), respectively; or (B) a VHCDR1 sequence, a VHCDR2 sequence, and a VHCDR3 sequence of GYYWS (SEQ ID NO: 23), EINHSGSTNYNPSLKS (SEQ ID NO: 24), and GPRGMYSSSSGDY (SEQ ID NO: 25), respectively; and a V_{L}CDR1 sequence, a V_{L}CDR2 sequence, and a V_{L}CDR3 sequence of: RASQGIRNDLG (SEQ ID NO: 26), AAVSLQS (SEQ ID NO: 27), and LQHNSYPRT (SEQ ID NO: 28), respectively; or (C) a VHCDR1 sequence, a VHCDR2 sequence, and a VHCDR3 sequence of TYGMH (SEQ ID NO: 29), VIWYDGSNTYYADSVKG (SEQ ID NO: 30), and ARGRGYSAQGNRNRAYYFDY (SEQ ID NO: 31) respectively; and a V_{L}CDR1 sequence, a V_{L}CDR2 sequence, and a V_{L}CDR3 sequence of: RASQSVSSNLA (SEQ ID NO: 32), GAFTRVT (SEQ ID NO: 33), and QQYNDRPPYT (SEQ ID NO: 34) respectively. In some embodiments, the nucleic acid encoding the receptor is operably linked to a promoter. The promoter may be a constitutive promoter or a conditional promoter. Additionally or alternatively, in some embodiments, the conditional promoter is induced by binding of the receptor to a U5 snRNP200 antigen. In certain embodiments, the U5 snRNP200 antigen binding fragment is an scFv, a Fab, or a F(ab)2.

Additionally or alternatively, in some embodiments, the U5 snRNP200 antigen binding fragment comprises a V_{H} amino acid sequence of any one of SEQ ID NOs: 35-37 and/or a V_{L} amino acid sequence of SEQ ID NOs: 38-40. In certain embodiments, the U5 snRNP200 antigen binding fragment comprises the V_{H} amino acid sequence and the V_{L} amino acid sequence of SEQ ID NO: 35 and SEQ ID NO: 38; SEQ ID NO: 36 and SEQ ID NO: 39; and SEQ ID NO: 37 and SEQ ID NO: 40, respectively.

In one aspect, the present disclosure provides an engineered immune cell including a receptor that comprises a U5 snRNP200 antigen binding fragment comprising an amino acid sequence selected from the group consisting of: SEQ ID NO: 2, SEQ ID NO: 6, SEQ ID NO: 8, SEQ ID NO: 10, SEQ ID NO: 12, and SEQ ID NO: 14; and/or a nucleic acid encoding the receptor. In some embodiments, the nucleic acid encoding the receptor is operably linked to a promoter. The promoter may be a constitutive promoter or a conditional promoter. Additionally or alternatively, in some embodiments, the conditional promoter is induced by binding of the receptor to a U5 snRNP200 antigen. In certain embodiments, the U5 snRNP200 antigen binding fragment is an scFv, a Fab, or a F(ab)2.

In any and all embodiments of the engineered immune cell disclosed herein, the receptor is a non-native cell receptor. In some embodiments, the receptor is a T cell receptor and/or a chimeric antigen receptor.

In any of the preceding embodiments of the engineered immune cell disclosed herein, the receptor is linked to a reporter or a selection marker, such as GFP or LNGFR or an IL-18 polypeptide. In some embodiments, the receptor is linked to the reporter or selection marker or the IL-18 polypeptide via a self-cleaving linker.

In any and all embodiments of the engineered immune cell disclosed herein, the chimeric antigen receptor comprises (i) an extracellular antigen binding domain; (ii) a transmembrane domain; and (iii) an intracellular domain. The extracellular antigen binding domain may comprise a signal peptide that is operably linked to the N-terminus of the extracellular antigen binding domain. In some embodiments, the extracellular antigen binding domain comprises a single chain variable fragment (scFv) or a human scFv. Additionally or alternatively, in some embodiments, the extracellular antigen binding domain comprises an anti-U5 snRNP200 scFv having at least 80%, 85%, 90%, 95%, 96%, 97%, 98%, or 99% sequence identity to any one of SEQ ID NOs: 2, 6, 8, 10, 12, or 14. In certain embodiments, the extracellular antigen binding domain comprises an anti-U5 snRNP200 scFv of any one of SEQ ID NOs: 2, 6, 8, 10, 12, or 14.

Additionally or alternatively, in some embodiments, the transmembrane domain comprises a CD8 transmembrane domain or a CD28 transmembrane domain and/or the intracellular domain comprises one or more costimulatory domains. Examples of costimulatory domains include, but are not limited to a CD28 costimulatory domain, a 4-1BB costimulatory domain, an OX40 costimulatory domain, an ICOS costimulatory domain, a DAP-10 costimulatory domain, a PD-1 costimulatory domain, a CTLA-4 costimulatory domain, a LAG-3 costimulatory domain, a 2B4 costimulatory domain, a BTLA costimulatory domain, a CD3-chain, or any combination thereof.

Additionally or alternatively, in some embodiments, the engineered immune cell of the present technology further comprises a signal peptide that is operably linked to the N-terminus of the IL-18 polypeptide. In certain embodiments, the signal peptide operably linked to the N-terminus of the IL-18 polypeptide comprises the amino acid sequence of SEQ ID NO: 79.

In any and all embodiments of the engineered immune cell described herein, the IL-18 polypeptide comprises the amino acid sequence of SEQ ID NO: 81. Additionally or alternatively, in some embodiments, the engineered immune cell of the present technology comprises a CD28 polypeptide comprising the amino acid sequence of SEQ ID NO: 74 or SEQ ID NO: 83.

In any and all embodiments of the engineered immune cell disclosed herein, the engineered immune cell is a lymphocyte, a T cell, a B cell, a natural killer (NK) cell, a CD4+ T cell, a CD8+ T cell or a tumor infiltrating lymphocyte. Additionally or alternatively, in some embodiments, the engineered immune cell is derived from an autologous donor or an allogenic donor.

In one aspect, the present disclosure provides a polypeptide comprising a chimeric antigen receptor comprising an amino acid sequence of any one of SEQ ID NOs: 2, 6, 8, 10, 12, 14, or 35-40. In certain embodiments, the polypeptide further comprises a self-cleaving peptide, such as a P2A self-cleaving peptide, located between the chimeric antigen receptor and a reporter or a selection marker or an IL-18 polypeptide. Additionally or alternatively, in some embodiments, the chimeric antigen receptor further comprises a leader sequence. In certain embodiments, the leader sequence comprises an amino acid sequence of SEQ ID NO: 41, SEQ ID NO: 43, SEQ ID NO: 45 or SEQ ID NO: 47.

In any and all embodiments of the polypeptide described herein, the chimeric antigen receptor comprises (i) an extracellular antigen binding domain; (ii) a transmembrane domain; and (iii) an intracellular domain. The extracellular antigen binding domain may comprise a signal peptide that is operably linked to the N-terminus of the extracellular antigen binding domain and/or binds to a U5 snRNP200 antigen. In some embodiments, the extracellular antigen binding domain comprises a single chain variable fragment (scFv) or a human scFv. Additionally or alternatively, in some embodiments, the extracellular antigen binding domain comprises an anti-U5 snRNP200 scFv having at least 80%, 85%, 90%, 95%, 96%, 97%, 98%, or 99% sequence identity to any one of SEQ ID NOs: 2, 6, 8, 10, 12, or 14. In certain embodiments, the extracellular antigen binding domain comprises an anti-U5 snRNP200 scFv of any one of SEQ ID NOs: 2, 6, 8, 10, 12, or 14.

Additionally or alternatively, in some embodiments, the transmembrane domain comprises a CD8 transmembrane domain or a CD28 transmembrane domain and/or the intracellular domain comprises one or more costimulatory domains. Examples of costimulatory domains include, but are not limited to a CD28 costimulatory domain, a 4-1BB costimulatory domain, an OX40 costimulatory domain, an ICOS costimulatory domain, a DAP-10 costimulatory domain, a PD-1 costimulatory domain, a CTLA-4 costimulatory domain, a LAG-3 costimulatory domain, a 2B4 costimulatory domain, a BTLA costimulatory domain, a CD32-chain, or any combination thereof.

In another aspect, the present disclosure provides a nucleic acid encoding any and all embodiments of the polypeptide disclosed herein. In some embodiments, the nucleic acid encoding the polypeptide is operably linked to a promoter, such as a constitutive promoter or a conditional promoter. In certain embodiments, the conditional promoter is inducible by the chimeric antigen receptor binding to a U5 snRNP200 antigen. Also disclosed herein are vectors comprising any and all embodiments of the nucleic acids described herein. The vector may be a viral vector, a plasmid, or a retroviral vector.

Also described herein are host cells comprising any and all embodiments of the nucleic acid or the vector disclosed herein. In another aspect, the present disclosure provides a kit comprising the engineered immune cell of the present technology, and instructions for use.

In one aspect, the present disclosure provides a method for preparing immune cells for cancer therapy comprising isolating immune cells from a donor subject; and transducing the immune cells with any and all embodiments of the nucleic acid or the vector disclosed herein. In another aspect, the present disclosure provides a method of treatment comprising isolating immune cells from a donor subject; transducing the immune cells with any and all embodiments of the nucleic acid or the vector disclosed herein; and administering the transduced immune cells to a recipient subject. In some embodiments, the donor subject and the recipient subject are the same. In other embodiments, the donor subject and the recipient subject are different. Additionally or alternatively, in some embodiments, the immune cells isolated from the donor subject comprise one or more lymphocytes. Examples of lymphocytes include, but are not limited to a T cell, a B cell, a natural killer (NK) cell, a CD4+ T cell, a CD8+ T cell or tumor infiltrating lymphocytes.

In yet another aspect, the present disclosure provides a method for treating cancer in a subject in need thereof comprising administering to the subject an effective amount of the engineered immune cells of the present technology. In some embodiments, the method further comprises administering to the subject a tumor specific monoclonal antibody. Also disclosed herein are methods for treating of inhibiting tumor growth or metastasis in a subject with cancer comprising contacting a tumor cell with an effective amount of the engineered immune cell of the present technology. In certain embodiments, the cancer is a relapsed or refractory cancer.

Additionally or alternatively, in some embodiments of the methods disclosed herein, the engineered immune cell is administered intravenously, intraperitoneally, subcutaneously, intramuscularly, or intratumorally.

In any of the preceding embodiments of the methods disclosed herein, the methods further comprise administering an additional cancer therapy. Examples of additional cancer therapy include chemotherapy, radiation therapy, immunotherapy, monoclonal antibodies, anti-cancer nucleic acids or proteins, anti-cancer viruses or microorganisms, and any combinations thereof.

Additionally or alternatively, in some embodiments of the methods disclosed herein, methods further comprise administering a cytokine to the subject. In certain embodiments, the cytokine is selected from the group consisting of interferon α, interferon β, interferon γ, complement C5a, IL-2, TNFalpha, CD40L, IL12, IL-23, IL15, IL17, CCL1, CCL11, CCL12, CCL13, CCL14-1, CCL14-2, CCL14-3, CCL15-1, CCL15-2, CCL16, CCL17, CCL18, CCL19, CCL19, CCL2, CCL20, CCL21, CCL22, CCL23-1, CCL23-2, CCL24, CCL25-1, CCL25-2, CCL26, CCL27, CCL28, CCL3, CCL3L1, CCL4, CCL4L1, CCL5, CCL6, CCL7, CCL8, CCL9, CCR10, CCR2, CCR5, CCR6, CCR7, CCR8, CCRL1, CCRL2, CX3CL1, CX3CR, CXCL1, CXCL10, CXCL11, CXCL12, CXCL13, CXCL14, CXCL15, CXCL16, CXCL2, CXCL3, CXCL4, CXCL5, CXCL6, CXCL7, CXCL8, CXCL9, CXCL9, CXCR1, CXCR2, CXCR4, CXCR5, CXCR6, CXCR7 and XCL2.

In any and all embodiments of the methods disclosed herein, the cancer or tumor is selected from among acute myeloid leukemia (AML), Diffuse large B-cell lymphoma (DLBCL), B-cell acute lymphoblastic leukemia (B-ALL), Follicular lymphoma, Chronic lymphocytic leukemia (CLL)/small lymphocytic lymphoma (SLL), Mantle cell lymphoma (MCL), Extranodal marginal zone B-cell lymphoma, Nodal marginal zone B-cell lymphoma, Splenic marginal zone B-cell lymphoma, Burkitt lymphoma, Lymphoplasmacytic lymphoma (Waldenstrom macroglobulinemia), Hairy cell leukemia, Primary central nervous system (CNS) lymphoma, lung cancer (e.g., non-small cell lung cancer), and metastases thereof.

Additionally or alternatively, in some embodiments, the methods further comprise sequentially, separately, or simultaneously administering to the subject at least one therapeutic agent. Examples of suitable therapeutic agents include, but are not limited to, cytarabine (cytosine arabinoside or ara-C), anthracycline drugs (such as daunorubicin (daunomycin) or idarubicin), cladribine (2-CdA), fludarabine, mitoxantrone, etoposide (VP-16), 6-thioguanine (6-TG), hydroxyurea, corticosteroid drugs (e.g., prednisone or dexamethasone), methotrexate (MTX), 6-mercaptopurine (6-MP), azacytidine, decitabine, FLT3 inhibitors (e.g., Midostaurin, Gilteritinib), IDH inhibitors (e.g., Ivosidenib, Olutasidenib, Enasidenib, Gemtuzumab ozogamicin), BCL-2 inhibitors (e.g., Venetoclax), Hedgehog pathway inhibitors (e.g., Glasdegib), Tafasitamab, blinatumomab, loncastuximab tesirine, Epratuzumab, Inotuzumab ozogamicin, hypomethylating agents, a Cytotoxic T-Lymphocyte Associated Protein 4 (CTLA4) inhibitor, a Programmed Cell Death 1 (PD-1) inhibitor, a Programmed Death Ligand 1 (PD-L1) inhibitor, BiTE/DART (e.g., Flotetuzumab, Vibecotamab), Antibody drug conjugates (e.g., IMGN632), Magrolimab, and MBG453+HMA.

### BRIEF DESCRIPTION OF THE DRAWINGS

FIGS. 1A-1E: High density immunophenotyping of acute myeloid leukemia (AML) surface antigen expression and co-expression patterns identifies U5 snRNP200 as an AML-associated antigen. FIG. 1A: Oncoprint summarizing AML patient characteristics, clinical parameters, and expression of AML-associated antigens on bone marrow leukemic blasts. FIG. 1B: Representative UMAPs comparing normal subject and age-matched AML patient bone marrow samples subjected to 36 parameter phenotyping. Heatmap colors indicate relative surface antigen expression intensity. Red dotted lined indicates unbiased identification of malignant blasts. FIG. 1C: Median fluorescent intensity (MFI) of surface antigen expression on normal bone marrow hematopoietic stem and progenitor cells versus AML blasts across patients. HSC-hematopoietic stem cell (Lin-CD⁻34⁺CD45^{dim}CD90⁺CD38⁻), MPP=multipotent progenitor (Lin⁻CD34⁺CD45^{dim}CD90⁻CD38⁻), CMP-common myeloid progenitor (Lin⁻CD34⁺CD45RA⁻CD38⁺CD123⁺), GMP=granulocyte-macrophage progenitor (Lin⁻CD34⁺CD45RA⁺CD38⁺CD123⁺), MEP=megakaryocyte-erythroid progenitor (Lin⁻CD34 CD45RA⁻CD38⁺CD123⁻); p from Mann-Whitney test: U5 snRNP200 Blasts vs. HSC ***p=0.0006, blasts vs. MPP ***p=0.0001, blasts vs. CMP **p=0.002, blasts vs. GMP **p=0.007, blasts vs. MEP ***p=0.0004; CD47 blasts vs. HSC ***p=0.0005, blasts vs. MPP **p=0.002, blasts vs. CMP *p=0.02, blasts vs. MEP *p=0.01; TIM-3 blasts vs. HSC *p=0.03, blasts vs. MPP **p=0.003, blasts vs. CMP *p=0.03. Bar graphs represent ±SEM. FIG. 1D: Circos plot depicting co-expression between U5 snRNP200 and known AML associated surface antigens on AML patient bone marrow blasts. FIG. 1E: Correlogram depicting Pearson Correlation coefficients of surface antigen intensity on AML patient bone marrow blasts. Spheres annotated with “1” show the highest positive correlation.

FIGS. 2A-2G: Validation of cell surface membrane U5 snRNP200 expression and CD32A regulation of U5 snRNP200 cell surface membrane localization. FIG. 2A: Schema of vector engineered for knockin of N-terminal HaloTag into the SNRNP200 locus in human AML cell line K562. Figure discloses SEQ ID NO: 86.

FIG. 2B: Western blot of HaloTag (two exposure times are shown and denoted as light and heavy) and U5 snRNP200 in sub-cellular fractions of K562 cell clones expressing HaloTagged endogenous U5 snRNP200. Loading controls for cell fractions include tubulin (cytoplasmic), sodium/potassium ATPase pump (membrane), and SP1 (soluble nuclear).

FIG. 2C: Representative flow cytometry histograms of MFI values (left) for cell impermeable (top panels) and permeable (bottom panels) fluorescent HaloTag ligands in K562 cells from FIG. 2B and quantification of signal (right). FIG. 2D: Schema of whole genome CRISPR screen to identify genes positively and negatively associated with U5 snRNP200 cell surface expression on AML cells. As shown the Brunello sgRNA library (via GFP⁺ lentivirus) was stably introduced in K562 and U937 cells and subsequently the top 10% and bottom 10% of U5 snRNP200 surface expressing GFP⁺ cells were sorted for sgRNA sequencing. FIG. 2E: Statistically significant sgRNAs associated with low U5 snRNP200 expression in U937 (y-axis) and K562 cells (x-axis). RRA=Robust Rank Aggregation. As shown, knockout of FCGR2A (which encodes CD32A) was significantly associated with low cell-surface snRNP200 expression across both cell lines. FIG. 2F: Gene ontology analysis of genes required for cell surface U5 snRNP200 expression from CRISPR screen in (FIG. 2E). FIG. 2G: Histograms of CD32A (left) and U5 snRNP200 (right) expression on U937 cells following stable knockout (KO) of CD32A and re-expression of CD32A using cDNA impervious to KO.

FIGS. 3A-3J: Fc engineered U5 snRNP200 antibody optimized for activation of immune cells demonstrates robust anti-leukemic effects. FIG. 3A: Histogram overlays of U5 snRNP200 surface expression on peripheral blood B-cells (left) and malignant myeloid cells (middle and right) by flow cytometry in murine genetically engineered models of AML. FIG. 3B: Schema of mouse inversion 3 AML model transplant and treatment schedule. FIG. 3C: Table describing mouse IgG Fc subclass binding to activating and inhibitory Fc receptors. FIG. 3D: Kaplan-Meier survival curve of recipient mice engrafted with inversion 3 AML cells following treatment with U5 snRNP200 antibody engineered with IgG2a Fc subclass or control (PBS). FIG. 3E: Kaplan-Meier survival curve of recipient mice engrafted with mouse RN2 (MLL-AF9 overexpression+NRAS^{G12D} mutation) cells following treatment with U5 snRNP200 antibody engineered with IgG2a Fc or IgG2b Fc or control. p from Log-rank test. **p= 0.001. FIG. 3F: Quantification of bioluminescent imaging comparing RN2 disease burden among control and U5 snRNP200 antibody treated mice. p from unpaired t-test; *p=0.02. FIG. 3G: Representative images of bioluminescent signal in control and U5 snRNP200 antibody treated mice. FIG. 3H: Kaplan-Meier survival curve of recipient mice engrafted with inversion 3 AML cells following treatment with U5 snRNP200 antibody engineered with IgG2a Fc subclass or control with or without concomitant azacitidine treatment (p from log-rank test; *p=0.01, **p=0.004, ****p<0.0001). FIG. 3I: Fc receptor Activating: Inhibitory ratios (CD16.2: CD32B) on peripheral blood CD45.2-monocytes/macrophages after 5 days of in vivo control or azacitidine treatment in inversion 3 engrafted mice. Bar graphs represent ±SEM (p from unpaired t-test; left *p=0.036, right *p=0.023). FIG. 3J: Representative expression histograms from individual mice in FIG. 3I.

FIG. 4: Generation of anti-U5 snRNP200 chimeric antigen receptors (CARs). U5 snRNP200 CARs include a single-chain Fragment variant (scFv), a co-stimulatory molecule CD28 and its transmembrane (TM) domain, and an intracellular T cell activation domain of CD35. scFv is tagged with the Myc sequence and is connected with mCherry via the P2A sequence. The sequences of scFv are derived from antibodies for U5 snRNP200 detected in AML patients (clones 23, 31, 37) (Marijn et al, Blood, 2018). In the generated H construct, the scFv antigen binding fragment are arranged in V_{H}-V_{L} order, and in the L construct, the scFv antigen binding fragment are arranged in V_{L}-V_{H} order.

FIG. 5: DNA and amino acid sequences of the 23H scFv region represented by SEQ ID NO: 1 and SEQ ID NO: 2, respectively. Heavy chain (blue sequence) and Light chain (red sequence) regions are connected by 15-amino acid GS linker GGGGSGGGGSGGGGS (SEQ ID NO: 3).

FIG. 6: DNA and amino acid sequences of the 23L scFv region represented by SEQ ID NO: 5 and SEQ ID NO: 6, respectively. Heavy chain (blue sequence) and Light chain (red sequence) regions are connected by 15-amino acid GS linker GGGGSGGGGSGGGGS (SEQ ID NO: 3).

FIG. 7: DNA and amino acid sequences of the 31H scFv region. represented by SEQ ID NO: 7 and SEQ ID NO: 8, respectively. Heavy chain (blue sequence) and Light chain (red sequence) regions are connected by 15-amino acid GS linker GGGGSGGGGSGGGGS (SEQ ID NO: 3).

FIG. 8: DNA and amino acid sequences of the 31L scFv region represented by SEQ ID NO: 9 and SEQ ID NO: 10, respectively. Heavy chain (blue sequence) and Light chain (red sequence) regions are connected by 15-amino acid GS linker

(SEQ ID NO: 3)
GGGGSGGGGSGGGGS.

FIG. 9: DNA and amino acid sequences of the 37H scFv region represented by SEQ ID NO: 11 and SEQ ID NO: 12, respectively. Heavy chain (blue sequence) and Light chain (red sequence) regions are connected by 15-amino acid GS linker

(SEQ ID NO: 3)
GGGGSGGGGSGGGGS.

FIG. 10: DNA and amino acid sequences of the 37 scFv region represented by SEQ ID NO: 13 and SEQ ID NO: 14, respectively. Heavy chain (blue sequence) and Light chain (red sequence) regions are connected by 15-amino acid GS linker

(SEQ ID NO: 3)
GGGGSGGGGSGGGGS.

FIG. 11: Anti-U5 snRNP200 CAR T constructs are successfully expressed on the cell surface of human T cells. Human T cells were transduced with U5 snRNP200 CAR T constructs. mCherry represents expression of CAR T constructs. Surface expression of U5 snRNP200 CARs was confirmed by staining with Myc antibodies. Non-transduced T cells that do not express mCherry nor Myc are used as negative control.

FIG. 12: Surface expression of U5 snRNP200 in AML cell lines. AML cell lines, including U937, OCI-AML2, OCI-AML3, and K562, were stained with PE-conjugated U5 snRNP200 23, 31, and 37 antibody clones.

FIG. 13: Anti-tumor effects of anti-U5 snRNP200 CAR T cells on AML cell lines. Anti-U5 snRNP200 CAR T cells were co-cultured with AML cell lines expressing firefly luciferase at the indicated test ratios (1:4-1:512). As tumor cells express luciferase, the cell viability can be assessed by the luciferase activity. 24 hours after co-culture, bioluminescence was measured to assess the cytotoxic activity of U5 snRNP200 CAR T cells on tumor cells. The cytotoxic effect is expressed as the signal reduction in tumor cells cocultured with U5 snRNP200 CAR T cells compared to those cocultured with non-targeting CAR T cells.

FIG. 14: Anti-U5 snRNP200 CAR T cells are proliferative and exhibit the cytotoxic activity against AML cells. Anti-U5 snRNP200 CAR T cells were co-cultured with U937 or OCI-AML2 tumor cells expressing enhanced GFP at the E:T ratio of 1:5 to perform the time-lapse imaging. The number of CAR T and tumor cells were assessed by counting mCherry-positive and GFP-positive cells, respectively over time.

FIG. 15: T cell receptor signaling is activated in anti-U5 snRNP200 CAR T cells. Anti-U5 snRNP200 CAR T cells were co-cultured with U937 or OCI-AML2 cells at the ratio of 1:5 for 48 hours. Cells were subsequently stained with anti-pAKT 1/2/3, anti-pERK 1/2, and anti-pZAP70 antibodies to evaluate the activity of T cell receptor signaling.

FIG. 16: Anti-U5 snRNP200 CAR T cells secrete multiple cytokines and effector molecules. Anti-U5 snRNP200 CAR T cells were co-cultured with U937 or OCI-AML2 cells at the ratio of 1:1. 24 hours after co-culture, the supernatant of culture media was collected for Luminex assay to conduct the cytokine profiling. The boxed area highlights treated samples that show the highest upregulation of all assayed cytokines.

FIG. 17: Surface expression of U5 snRNP200 in primary AML cells. AML patient-derived tumor cells (AML-60B and AML-133C) were stained with PE-conjugated anti-U5 snRNP200 23, 31, and 37 antibody clones. Cells derived from both patients express U5 snRNP200 on the cell surface. Both patients are therapy refractory cases.

FIG. 18: Cytotoxic activity of anti-U5 snRNP200 CAR T cells against primary AML cells. Anti-U5 snRNP200 CAR T cells (2 donors for AML-60B and 1 donor for AML-133C) were co-cultured with primary AML cells (AML-60B and AML-133C) at the ratio of 1:5 for 7 days. The number of CAR T and tumor cells were counted. (Left panel) Fold changes in the number of U5 snRNP200 CAR T cells compared with non-targeting CAR T cells (5DEL) are shown. (Right panel) Fold changes in the number of tumor cells co-cultured with U5 snRNP200 CAR T cells compared with those co-cultured with non-targeting CAR T cells (5DEL) are shown. CAR T cells expressing 23H, 37H, and 37L scFvs are highly proliferative and effective on tumor cells.

FIG. 19: Surface expression of U5 snRNP200 in B cell malignancies. DLBCL and B-ALL cells were stained with PE-conjugated anti-U5 snRNP200 23, 31, and 37 antibody clones.

FIGS. 20A-20B show cytotoxic effects of anti-U5 snRNP200 CAR T cells on DLBCL cell lines as determined using 24 hour killing assay. Anti-U5 snRNP200 CAR T cells were co-cultured with DLBCL cells expressing firefly luciferase at the indicated ratio. 24 hours after co-culture, bioluminescence was measured to assess the cytotoxic activity of anti-U5 snRNP200 CAR T cells on tumor cells. The cytotoxic effect is expressed as the signal reduction in tumor cells co-cultured with anti-U5 snRNP200 CAR T cells compared to those co-cultured with non-targeting CAR T cells. Anti-CD19 CAR T cells were used as positive control in FIG. 20A.

FIGS. 20C-20D show cytotoxic effects of anti-U5 snRNP200 CAR T cells on DLBCL cell lines as determined using IncuCyte assay. Anti-U5 snRNP200 CAR T cells were co-cultured with NALM6 (B-ALL), TMD8 (DLBCL), or RS4; 11 (B-ALL) cells expressing enhanced GFP at the E:T ratio of 1:2 (NALM6), 2:1 (TMD8), or 1:1 (RS4; 11) to perform the time-lapse imaging. The number of CAR T and tumor cells were assessed by counting mCherry-positive and GFP-positive cells, respectively over time. Anti-CD19 CAR T cells were used as positive control.

FIG. 21: Anti-tumor effect of anti-U5 snRNP200 CAR T cells on U937 human AML cells in vivo. (Upper panel) AML cell lines were stained with PE-conjugated anti-U5 snRNP200 23 and 37 antibody clones. (Lower panel) Anti-U5 snRNP200 CAR T cells and U937 cells expressing firefly luciferase were transplanted into immunodeficient NOD-Prkdc^{em26Cd52}Il2rg^{em26Cd22}/NjuCrl (NCG) mice. In vivo bioluminescence imaging was performed on the indicated days after transplantation of U937 cells.

FIG. 22: Cytotoxic/anti-tumor effects of anti-U5 snRNP200 CAR T cells on DLBCL cell lines. Anti-U5 snRNP200 CAR T cells were co-cultured with DLBCL cells expressing firefly luciferase at the indicated ratio. 24 hours after co-culture, bioluminescence was measured to assess the cytotoxic activity of anti-U5 snRNP200 CAR T cells on tumor cells. The cytotoxic effect is expressed as the signal reduction in tumor cells co-cultured with anti-U5 snRNP200 CAR T cells compared to those co-cultured with non-targeting CAR T cells. Anti-CD19 CAR T cells were used as positive control. CAR T cells exhibit anti-tumor effects on DLBCL cell lines. Anti-U5 snRNP200 CAR T cells exhibit anti-tumor effects on DLBCL cell lines.

FIGS. 23A-23C: Development of armored, IL-18 secreting anti-U5 snRNP200 CAR-Ts and their preclinical activity in acute myeloid leukemia.

FIGS. 24A-24D: Application of anti-U5 snRNP CAR-T cells to B-cell malignancies and non-small cell lung cancer.

### DETAILED DESCRIPTION

It is to be appreciated that certain aspects, modes, embodiments, variations and features of the present methods are described below in various levels of detail in order to provide a substantial understanding of the present technology.

The present disclosure is not to be limited in terms of the particular embodiments described in this application, which are intended as single illustrations of individual aspects of the disclosure. All the various embodiments of the present disclosure will not be described herein. Many modifications and variations of the disclosure can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that the present disclosure is not limited to particular uses, methods, reagents, compounds, compositions or biological systems, which can, of course, vary. It is also to be understood that the terminology used herein is for the purpose of describing particular embodiments only, and is not intended to be limiting.

In practicing the present methods, many conventional techniques in molecular biology, protein biochemistry, cell biology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

Currently there are no effective immunotherapies for AML, and this is, in part, due to a lack of known antigens which are unique to AML and not present on normal hematopoietic precursors. As described herein, high density immunophenotyping of acute myeloid leukemia (AML) surface antigen expression and co-expression patterns identified U5 snRNP200 as an AML-associated antigen. U5 snRNP200 is a ubiquitously expressed nuclear RNA helicase and core component of the spliceosome. It is therefore unexpected that it would translocate to the cell membrane. As described in the Examples herein, the cell surface localization of U5 snRNP200 was validated and its mechanism of cell surface trafficking in AML cells was delineated. While U5 snRNP200 is absent from normal hematopoietic stem and progenitor cells, interestingly it is expressed on all B-cells (a previously unknown finding).

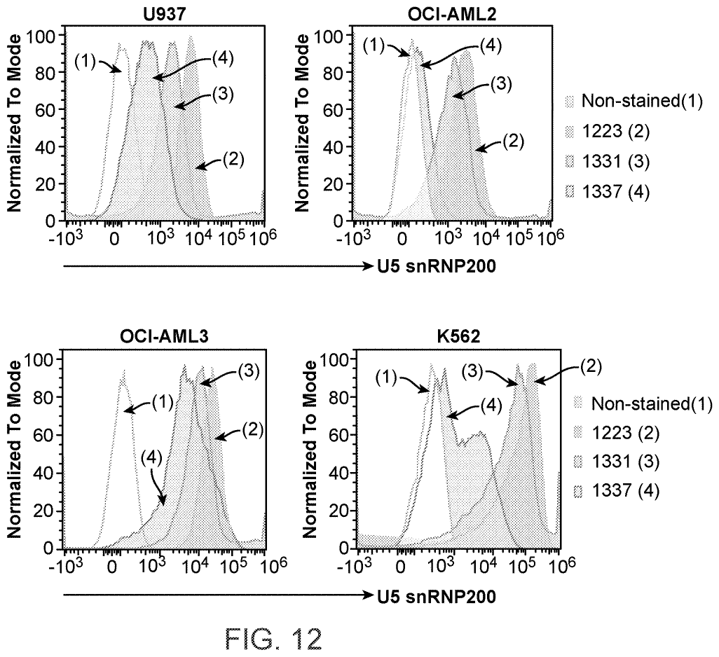

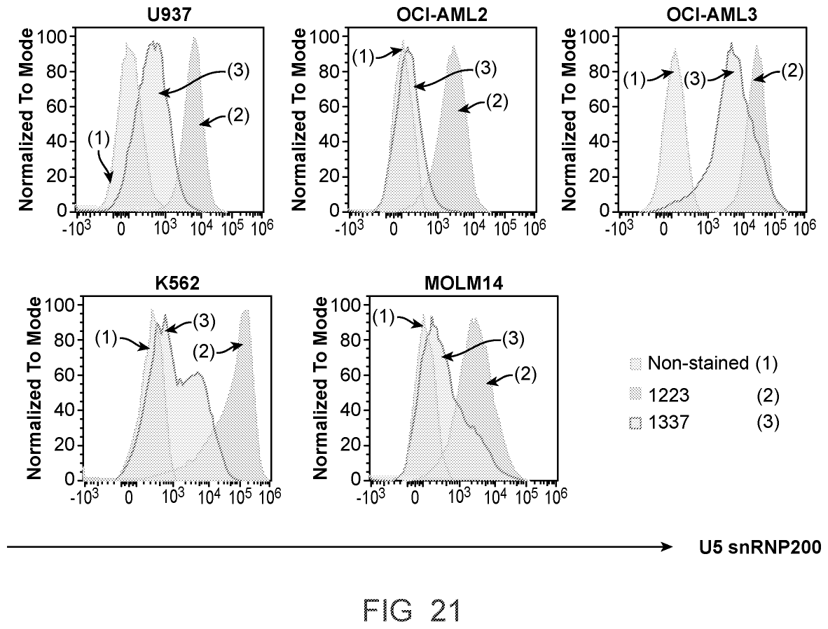

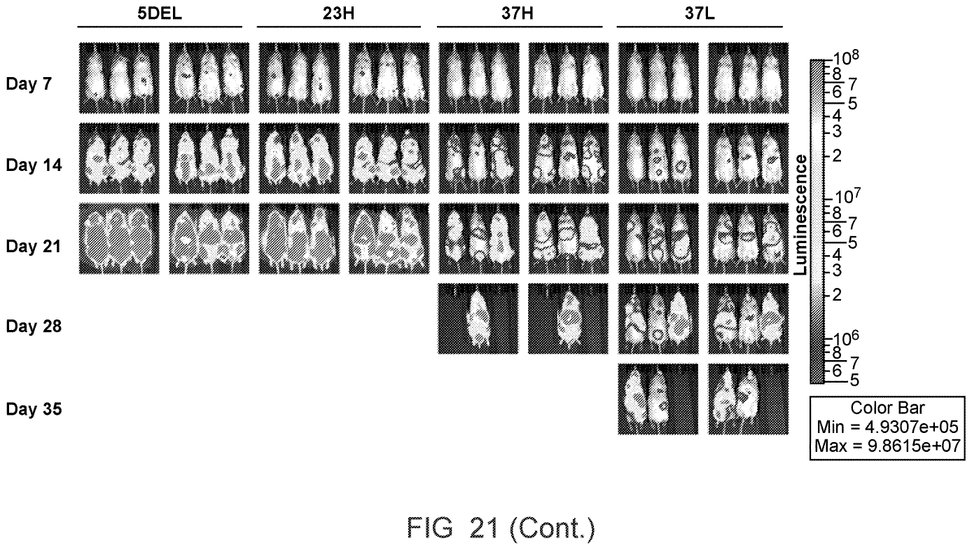

Moreover, the clone 37 anti-U5 snRNP200 CAR T cells showed superior in vivo anti-tumor efficacy and survival compared with the clone 23 anti-U5 snRNP200 CAR T cells 21 days after transplantation of AML cells (FIG. 21), even though clone 23 showed higher binding in AML cells (FIG. 12).

### Definitions

As it would be understood, the section or subsection headings as used herein is for organizational purposes only and are not to be construed as limiting and/or separating the subject matter described.

Unless defined otherwise, all technical and scientific terms used herein have the meaning commonly understood by a person skilled in the art to which this disclosure belongs. The following references provide one of skill with a general definition of many of the terms used in the present disclosure. Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise. The terminology used herein is for the purpose of describing particular embodiments only and is not intended to be limiting of the disclosure.

As used herein, the term “comprising” is intended to mean that the compounds, compositions and methods include the recited elements, but not exclude others. “Consisting essentially of” when used to define compounds, compositions and methods, shall mean excluding other elements of any essential significance to the combination. Thus, a composition consisting essentially of the elements as defined herein would not exclude trace contaminants, e.g., from the isolation and purification method and pharmaceutically acceptable carriers, preservatives, and the like. “Consisting of” shall mean excluding more than trace elements of other ingredients. Embodiments defined by each of these transition terms are within the scope of this technology.

All numerical designations, e.g., pH, temperature, time, concentration, and molecular weight, including ranges, are approximations which are varied (+) or (−) by increments of 1.0 or 0.1, as appropriate or alternatively by a variation of +/−20% or +/−15%, or alternatively 10% or alternatively 5% or alternatively 2%. As will be understood by one skilled in the art, for any and all purposes, all ranges disclosed herein also encompass any and all possible subranges and combinations of subranges thereof. Furthermore, as will be understood by one skilled in the art, a range includes each individual member.

As used herein, the singular forms “a”, “an” and “the” are intended to include the plural forms as well, unless the context clearly indicates otherwise. For example, the term “a cell” includes a plurality of cells, including mixtures thereof.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, or within 2-fold, of a value.

As used herein, the term “administration” of an agent to a subject includes any route of introducing or delivering the agent to a subject to perform its intended function. Administration can be carried out by any suitable route, including, but not limited to, intravenously, intramuscularly, intraperitoneally, subcutaneously, and other suitable routes as described herein. Administration includes self-administration and the administration by another. “Administration” of a cell or vector or other agent and compositions containing same can be performed in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician or in the case of animals, by the treating veterinarian. In some embodiments, administering or a grammatical variation thereof also refers to more than one doses with certain interval. In some embodiments, the interval is 1 day, 2 days, 3 days, 4 days, 5 days, 6 days, 1 week, 10 days, 2 weeks, 3 weeks, 1 month, 2 months, 3 months, 4 months, 5 months, 6 months, 1 year or longer. In some embodiments, one dose is repeated for once, twice, three times, four times, five times, six times, seven times, eight times, nine times, ten times or more. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include oral administration, intraperitoneal, infusion, nasal administration, inhalation, injection, and topical application. In some embodiments, the administration is an infusion (for example to peripheral blood of a subject) over a certain period of time, such as about 30 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 24 hours or longer.

As used herein “adoptive cell therapeutic composition” refers to any composition comprising cells suitable for adoptive cell transfer. In exemplary embodiments, the adoptive cell therapeutic composition comprises a cell type selected from a group consisting of a tumor infiltrating lymphocyte (TIL), TCR (i.e. heterologous T-cell receptor) modified lymphocytes (e.g., eTCR T cells and caTCR T cells) and CAR (i.e. chimeric antigen receptor) modified lymphocytes (e.g., CAR T cells). In another embodiment, the adoptive cell therapeutic composition comprises a cell type selected from a group consisting of T-cells, CD8+ cells, CD4+ cells, NK-cells, delta-gamma T-cells, regulatory T-cells and peripheral blood mononuclear cells. In another embodiment, TILs, T-cells, CD8+ cells, CD4+ cells, NK-cells, delta-gamma T-cells, regulatory T-cells or peripheral blood mononuclear cells form the adoptive cell therapeutic composition. In one embodiment, the adoptive cell therapeutic composition comprises T cells.

The term “amino acid” refers to naturally occurring and non-naturally occurring amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrolysine and selenocysteine. Amino acid analogs refer to agents that have the same basic chemical structure as a naturally occurring amino acid, i.e., an a carbon that is bound to a hydrogen, a carboxyl group, an amino group, and an R group, such as, homoserine, norleucine, methionine sulfoxide, methionine methyl sulfonium. Such analogs have modified R groups (such as, norleucine) or modified peptide backbones, but retain the same basic chemical structure as a naturally occurring amino acid. In some embodiments, amino acids forming a polypeptide are in the D form. In some embodiments, the amino acids forming a polypeptide are in the L form. In some embodiments, a first plurality of amino acids forming a polypeptide are in the D form, and a second plurality of amino acids are in the L form.

Amino acids are referred to herein by either their commonly known three letter symbols or by the one-letter symbols recommended by the IUPAC-IUB Biochemical Nomenclature Commission. Nucleotides, likewise, are referred to by their commonly accepted single-letter code.

As used herein, the term “analog” refers to a structurally related polypeptide or nucleic acid molecule having the function of a reference polypeptide or nucleic acid molecule.

As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments that lack the Fc fragment of intact antibody, clear more rapidly from the circulation, and may have less non-specific tissue binding of an intact antibody (Wahl et al., J. Nucl. Med. 24:316-325 (1983)). Antibodies may comprise whole native antibodies, monoclonal antibodies, human antibodies, humanized antibodies, camelised antibodies, multispecific antibodies, bispecific antibodies, chimeric antibodies, Fab, Fab′, single chain V region fragments (scFv), single domain antibodies (e.g., nanobodies and single domain camelid antibodies), VNAR fragments, Bi-specific T-cell engager (BiTE) antibodies, minibodies, disulfide-linked Fvs (sdFv), and anti-idiotypic (anti-Id) antibodies, intrabodies, fusion polypeptides, unconventional antibodies and antigen binding fragments of any of the above. In particular, antibodies include immunoglobulin molecules and immunologically active fragments of immunoglobulin molecules, i.e., molecules that contain an antigen binding site. Immunoglobulin molecules can be of any type (e.g., IgG, IgE, IgM, IgD, IgA, and IgY), class (e.g., IgG1, IgG2, IgG3, IgG4, IgAQ1, and IgA2), or subclass.

In certain embodiments, an antibody is a glycoprotein comprising at least two heavy (H) chains and two light (L) chains inter-connected by disulfide bonds. Each heavy chain is comprised of a heavy chain variable region (abbreviated herein as V_{H}) and a heavy chain constant (C_{H}) region. The heavy chain constant region is comprised of three domains, C_{H}1, C_{H}2, and C_{H}3. Each light chain is comprised of a light chain variable region (abbreviated herein as V_{L}) and a light chain constant C_{L} region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. As used herein interchangeably, the terms “antigen binding portion”, “antigen binding fragment”, or “antigen binding region” of an antibody, refer to the region or portion of an antibody that binds to the antigen and which confers antigen specificity to the antibody; fragments of antigen binding proteins, for example antibodies, include one or more fragments of an antibody that retain the ability to specifically bind to an antigen. It has been shown that the antigen binding function of an antibody can be performed by fragments of a full-length antibody. Examples of antigen binding portions encompassed within the term “antibody fragments” of an antibody include a Fab fragment, a monovalent fragment consisting of the V_{L}, V_{H}, C_{L} and C_{H}1 domains; a F(ab)₂ fragment, a bivalent fragment comprising two Fab fragments linked by a disulfide bridge at the hinge region; a Fd fragment consisting of the V_{H} and C_{H}1 domains; a Fv fragment consisting of the V_{L} and V_{H} domains of a single arm of an antibody; a dAb fragment (Ward et al., Nature 341:544-546 (1989)), which consists of a V_{H} domain; and an isolated complementarity determining region (CDR). An “isolated antibody” or “isolated antigen binding protein” is one which has been identified and separated and/or recovered from a component of its natural environment. “Synthetic antibodies” or “recombinant antibodies” are generally generated using recombinant technology or using peptide synthetic techniques known to those of skill in the art.

Antibodies and antibody fragments can be wholly or partially derived from mammals (e.g., humans, non-human primates, goats, guinea pigs, hamsters, horses, mice, rats, rabbits and sheep) or non-mammalian antibody producing animals (e.g., chickens, ducks, geese, snakes, and urodele amphibians). The antibodies and antibody fragments can be produced in animals or produced outside of animals, such as from yeast or phage (e.g., as a single antibody or antibody fragment or as part of an antibody library).

Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules. These are known as single chain Fv (scFv); see e.g., Bird et al., Science 242:423-426 (1988); and Huston et al., Proc. Natl. Acad. Sci. 85:5879-5883 (1988). These antibody fragments are obtained using conventional techniques known to those of ordinary skill in the art, and the fragments are screened for utility in the same manner as are intact antibodies.

As used herein, the term “single-chain variable fragment” or “scFv” is a fusion protein of the variable regions of the heavy (V_{H}) and light chains (V_{L}) of an immunoglobulin (e.g., mouse or human) covalently linked to form a V_{H}::V_{L} heterodimer. The heavy (V_{H}) and light chains (V_{L}) are either joined directly or joined by a peptide-encoding linker (e.g., about 10, 15, 20, 25 amino acids), which connects the N-terminus of the V_{H} with the C-terminus of the V_{L}, or the C-terminus of the V_{H} with the N-terminus of the V_{L}. The linker is usually rich in glycine for flexibility, as well as serine or threonine for solubility. The linker can link the heavy chain variable region and the light chain variable region of the extracellular antigen binding domain. In certain embodiments, the linker comprises amino acids having GGGGSGGGGSGGGGS (SEQ ID NO: 3). In certain embodiments, the nucleic acid sequence encoding the amino acid sequence of SEQ ID NO: 3 is ggcggcggcggatctggaggtggtggctcaggtggcggaggctcc (SEQ ID NO: 4).

Despite removal of the constant regions and the introduction of a linker, scFv proteins retain the specificity of the original immunoglobulin. Single chain Fv polypeptide antibodies can be expressed from a nucleic acid comprising V_{H}- and V_{L}-encoding sequences as described by Huston, et al. (Proc. Nat. Acad. Sci. USA, 85:5879-5883 (1988)). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hybridoma (Larchmt) 27 (6): 455-51 (2008); Peter et al., J Cachexia Sarcopenia Muscle (2012); Shieh et al., J Imunol 183 (4): 2277-85 (2009); Giomarelli et al., Thromb Haemost 97 (6): 955-63 (2007); Fife eta., J Clin Invst 116 (8): 2252-61 (2006); Brocks et al., Immunotechnology 3 (3): 173-84 (1997); Moosmayer et al., Ther Immunol 2 (10): 31-40 (1995). Agonistic scFvs having stimulatory activity have been described (see, e.g., Peter et al., J Biol Chem 25278 (38): 36740-7 (2003); Xie et al., Nat Biotech 15 (8): 768-71 (1997); Ledbetter et al., Crit Rev Immunol 17 (5-6): 427-55 (1997); Ho et al., Bio Chim Biophys Acta 1638 (3): 257-66 (2003)).

As used herein, an “antigen” refers to a molecule to which an antibody can selectively bind. The target antigen may be a protein (e.g., an antigenic peptide), carbohydrate, nucleic acid, lipid, hapten, or other naturally occurring or synthetic compound. An antigen may also be administered to an animal subject to generate an immune response in the subject.

By “binding affinity” is meant the strength of the total noncovalent interactions between a single binding site of a molecule (e.g., an antibody) and its binding partner (e.g., an antigen). Without wishing to be bound by theory, affinity depends on the closeness of stereochemical fit between antibody combining sites and antigen determinants, on the size of the area of contact between them, and on the distribution of charged and hydrophobic groups. Affinity also includes the term “avidity,” which refers to the strength of the antigen-antibody bond after formation of reversible complexes (e.g., either monovalent or multivalent). Methods for calculating the affinity of an antibody for an antigen are known in the art, comprising use of binding experiments to calculate affinity. The affinity of a molecule X for its partner Y can generally be represented by the dissociation constant (K_{d}). A low-affinity complex contains an antibody that generally tends to dissociate readily from the antigen, whereas a high-affinity complex contains an antibody that generally tends to remain bound to the antigen for a longer duration. Antibody activity in functional assays (e.g., flow cytometry assay) is also reflective of antibody affinity. Antibodies and affinities can be phenotypically characterized and compared using functional assays (e.g., flow cytometry assay).

As used herein, “CDRs” are defined as the complementarity determining region amino acid sequences of an antibody which are the hypervariable regions of immunoglobulin heavy and light chains. See, e.g., Kabat et al., Sequences of Proteins of Immunological Interest, 4th U. S. Department of Health and Human Services, National Institutes of Health (1987). Generally, antibodies comprise three heavy chain and three light chain CDRs or CDR regions in the variable region. CDRs provide the majority of contact residues for the binding of the antibody to the antigen or epitope. In certain embodiments, the CDRs regions are delineated using the Kabat system (Kabat, E. A., et al. Sequences of Proteins of Immunological Interest, Fifth Edition, U.S. Department of Health and Human Services, NIH Publication No. 91-3242 (1991)).

As used herein, a “cancer” is a disease state characterized by the presence in a subject of cells demonstrating abnormal uncontrolled replication and in some aspects, the term may be used interchangeably with the term “tumor.” The term “cancer or tumor antigen” refers to an antigen known to be associated and expressed in a cancer cell or tumor cell (such as on the cell surface) or tissue, and the term “cancer or tumor targeting antibody” refers to an antibody that targets such an antigen. In some embodiments, the cancer or tumor antigen is not expressed in a non-cancer cell or tissue. In some embodiments, the cancer or tumor antigen is expressed in a non-cancer cell or tissue at a level significantly lower compared to a cancer cell or tissue. In some embodiments, the cancer is AML or a B cell malignancy (e.g., Diffuse large B-cell lymphoma (DLBCL), B-cell acute lymphoblastic leukemia (B-ALL), Follicular lymphoma, Chronic lymphocytic leukemia (CLL)/small lymphocytic lymphoma (SLL), Mantle cell lymphoma (MCL), Extranodal marginal zone B-cell lymphoma, Nodal marginal zone B-cell lymphoma, Splenic marginal zone B-cell lymphoma, Burkitt lymphoma, Lymphoplasmacytic lymphoma (Waldenstrom macroglobulinemia), Hairy cell leukemia, Primary central nervous system (CNS) lymphoma) or lung cancer (e.g., non-small cell lung cancer). In some embodiments, the cancer is a primary cancer or a metastatic cancer. In some embodiments, the cancer is a relapsed cancer. In some embodiments, the cancer reaches a remission, but can relapse. In some embodiments, the cancer is unresectable.

As used herein, the term “cell population” refers to a group of at least two cells expressing similar or different phenotypes. In non-limiting examples, a cell population can include at least about 10, at least about 100, at least about 200, at least about 300, at least about 400, at least about 500, at least about 600, at least about 700, at least about 800, at least about 900, at least about 1000 cells, at least about 10,000 cells, at least about 100,000 cells, at least about 1×10⁶ cells, at least about 1×10⁷ cells, at least about 1×10⁸ cells, at least about 1×10⁹ cells, at least about 1×10¹⁰ cells, at least about 1×10¹¹ cells, at least about 1×10¹² cells, or more cells expressing similar or different phenotypes.

As used herein, the term “chimeric co-stimulatory receptor” or “CCR” refers to a chimeric receptor that binds to an antigen and provides co-stimulatory signals, but does not provide a T-cell activation signal.

As used herein, a “cleavable peptide”, which is also referred to as a “cleavable linker,” means a peptide that can be cleaved, for example, by an enzyme. One translated polypeptide comprising such cleavable peptide can produce two final products, therefore, allowing expressing more than one polypeptides from one open reading frame. One example of cleavable peptides is a self-cleaving peptide, such as a 2A self-cleaving peptide. 2A self-cleaving peptides, is a class of 18-22 aa-long peptides, which can induce the cleaving of the recombinant protein in a cell. In some embodiments, the 2A self-cleaving peptide is selected from P2A, T2A, E2A, F2A and BmCPV2A. See, for example, Wang Y, et al. Sci Rep. 2015; 5:16273. Published 2015 Nov. 5. As used herein, the terms “T2A” and “2A peptide” are used interchangeably to refer to any 2A peptide or fragment thereof, any 2A-like peptide or fragment thereof, or an artificial peptide comprising the requisite amino acids in a relatively short peptide sequence (on the order of 20 amino acids long depending on the virus of origin) containing the consensus polypeptide motif D-V/I-E-X-N-P-G-P (SEQ ID NO: 15), wherein X refers to any amino acid generally thought to be self-cleaving.

As used herein, “complementary” sequences refer to two nucleotide sequences which, when aligned anti-parallel to each other, contain multiple individual nucleotide bases which pair with each other. Paring of nucleotide bases forms hydrogen bonds and thus stabilizes the double strand structure formed by the complementary sequences. It is not necessary for every nucleotide base in two sequences to pair with each other for sequences to be considered “complementary”. Sequences may be considered complementary, for example, if at least 30%, 40%, 50%, 55%, 60%, 65%, 70%, 75%, 80%, 85%, 90%, 95%, 98%, 99%, or 100% of the nucleotide bases in two sequences pair with each other. In some embodiments, the term complementary refers to 100% of the nucleotide bases in two sequences pair with each other. In addition, sequences may still be considered “complementary” when the total lengths of the two sequences are significantly different from each other. For example, a primer of 15 nucleotides may be considered “complementary” to a longer polynucleotide containing hundreds of nucleotides if multiple individual nucleotide bases of the primer pair with nucleotide bases in the longer polynucleotide when the primer is aligned anti-parallel to a particular region of the longer polynucleotide. Nucleotide bases paring is known in the field, such as in DNA, the purine adenine (A) pairs with the pyrimidine thymine (T) and the pyrimidine cytosine (C) always pairs with the purine guanine (G); while in RNA, adenine (A) pairs with uracil (U) and guanine (G) pairs with cytosine (C). Further, the nucleotide bases aligned anti-parallel to each other in two complementary sequences, but not a pair, are referred to herein as a mismatch.

A “composition” is intended to mean a combination of active agent and another compound or composition, inert (for example, a nanoparticle, detectable agent or label) or active, such as an adjuvant, diluent, binder, stabilizer, buffers, salts, lipophilic solvents, preservative, adjuvant or the like and include carriers, such as pharmaceutically acceptable carriers. In some embodiments, the carrier (such as the pharmaceutically acceptable carrier) comprises, or consists essentially of, or yet further consists of a nanoparticle, such as an polymeric nanoparticle carrier or an lipid nanoparticle that can be used alone or in combination with another carrier, such as an adjuvant or solvent. Carriers also include pharmaceutical excipients and additives proteins, peptides, amino acids, lipids, and carbohydrates (e.g., sugars, including monosaccharides, di-, tri, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol. A composition as disclosed herein can be a pharmaceutical composition. A “pharmaceutical composition” is intended to include the combination of an active agent with a carrier, inert or active, making the composition suitable for diagnostic or therapeutic use in vitro, in vivo or ex vivo.

As used herein, the term “conservative sequence modification” refers to an amino acid modification that does not significantly affect or alter the binding characteristics of the presently disclosed CAR (e.g., the extracellular antigen binding domain of the CAR) comprising the amino acid sequence. Conservative modifications can include amino acid substitutions, additions, and deletions. Modifications can be introduced into the human scFv of the presently disclosed CAR by standard techniques known in the art, such as site-directed mutagenesis and PCR-mediated mutagenesis. Amino acids can be classified into groups according to their physicochemical properties such as charge and polarity.

Conservative amino acid substitutions are ones in which the amino acid residue is replaced with an amino acid within the same group. For example, amino acids can be classified by charge: positively-charged amino acids include lysine, arginine, histidine; negatively-charged amino acids include aspartic acid and glutamic acid; and neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. Thus, one or more amino acid residues within a CDR region can be replaced with other amino acid residues from the same group and the altered antibody can be tested for retained function (i.e., the functions set forth in (c) through (1) above) using the functional assays described herein. In certain embodiments, no more than one, no more than two, no more than three, no more than four, no more than five residues within a specified sequence or a CDR region are altered.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a composition known to exhibit the desired therapeutic effect) and a negative control (a subject or a sample that does not receive the therapy or receives a placebo) are typically employed.

As used herein, the term, “co-stimulatory signaling domain,” or “co-stimulatory domain”, refers to the portion of the CAR comprising the intracellular domain of a co-stimulatory molecule. Co-stimulatory molecules are cell surface molecules other than antigen receptors or Fc receptors that provide a second signal required for efficient activation and function of T lymphocytes upon binding to antigen. Examples of such co-stimulatory molecules include CD27, CD28, 4-1BB (CD137), OX40 (CD134), CD30, CD40, PD-1, ICOS (CD278), LFA-1, CD2, CD7, LIGHT, NKD2C, B7-H2 and a ligand that specifically binds CD83. Accordingly, while the present disclosure provides exemplary costimulatory domains derived from CD28 and 4-1BB, other costimulatory domains are contemplated for use with the CARs described herein. The inclusion of one or more co-stimulatory signaling domains can enhance the efficacy and expansion of T cells expressing CAR receptors. The intracellular signaling and co-stimulatory signaling domains can be linked in any order in tandem to the carboxyl terminus of the transmembrane domain.

As used herein, the phrase “derived” means isolated, purified, mutated, or engineered, or any combination thereof. For example, an immune cell derived from a donor refers to the immune cell isolated from a biological sample of the donor and optionally engineered.

As used herein, the term “effective amount” or “therapeutically effective amount” refers to a quantity of an agent sufficient to achieve a beneficial or desired clinical result upon treatment. In the context of therapeutic applications, the amount of a therapeutic agent administered to the subject can depend on the type and severity of the disease or condition and on the characteristics of the individual, such as general health, age, sex, body weight, effective concentration of the engineered immune cells administered, and tolerance to drugs. It can also depend on the degree, severity, and type of disease. The skilled artisan will be able to determine appropriate dosages depending on these and other factors. An effective amount can be administered to a subject in one or more doses. In terms of treatment, an effective amount is an amount that is sufficient to palliate, ameliorate, stabilize, reverse or slow the progression of the disease, or otherwise reduce the pathological consequences of the disease. The effective amount is generally determined by the physician on a case-by-case basis and is within the skill of one in the art.

As used herein, the term “excipient” refers to a natural or synthetic substance formulated alongside the active ingredient of a medication, included for the purpose of long-term stabilization, bulking up solid formulations, or to confer a therapeutic enhancement on the active ingredient in the final dosage form, such as facilitating drug absorption, reducing viscosity, or enhancing solubility.

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression can include splicing of the mRNA in a eukaryotic cell. The expression level of a gene can be determined by measuring the amount of mRNA or protein in a cell or tissue sample. In one aspect, the expression level of a gene from one sample can be directly compared to the expression level of that gene from a control or reference sample. In another aspect, the expression level of a gene from one sample can be directly compared to the expression level of that gene from the same sample following administration of the compositions disclosed herein. The term “expression” also refers to one or more of the following events: (1) production of an RNA template from a DNA sequence (e.g., by transcription) within a cell; (2) processing of an RNA transcript (e.g., by splicing, editing, 5′ cap formation, and/or 3′ end formation) within a cell; (3) translation of an RNA sequence into a polypeptide or protein within a cell; (4) post-translational modification of a polypeptide or protein within a cell; (5) presentation of a polypeptide or protein on the cell surface; and (6) secretion or presentation or release of a polypeptide or protein from a cell. The level of expression of a polypeptide can be assessed using any method known in art, including, for example, methods of determining the amount of the polypeptide produced from the host cell. Such methods can include, but are not limited to, quantitation of the polypeptide in the cell lysate by ELISA, Coomassie blue staining following gel electrophoresis, Lowry protein assay and Bradford protein assay.

As used herein, an “expression vector” includes vectors capable of expressing DNA that is operably linked with regulatory sequences, such as promoter regions, that are capable of effecting expression of such DNA fragments. Such additional segments can include promoter and terminator sequences, and optionally can include one or more origins of replication, one or more selectable markers, an enhancer, a polyadenylation signal, and the like. Expression vectors are generally derived from plasmid or viral DNA, or can contain elements of both. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome.

As used herein, “F(ab)” refers to a fragment of an antibody structure that binds to an antigen but is monovalent and does not have a Fc portion, for example, an antibody digested by the enzyme papain yields two F(ab) fragments and an Fc fragment (e.g., a heavy (H) chain constant region; Fc region that does not bind to an antigen).

As used herein, “F(ab′)₂” refers to an antibody fragment generated by pepsin digestion of whole IgG antibodies, wherein this fragment has two antigen binding (ab′) (bivalent) regions, wherein each (ab′) region comprises two separate amino acid chains, a part of a H chain and a light (L) chain linked by an S—S bond for binding an antigen and where the remaining H chain portions are linked together. A “F(ab′)2” fragment can be split into two individual Fab′ fragments.

As used herein, the term “heterologous nucleic acid molecule or polypeptide” refers to a nucleic acid molecule (e.g., a cDNA, DNA or RNA molecule) or polypeptide that is either not normally expressed or is expressed at an aberrant level in a cell or sample obtained from a cell. This nucleic acid can be from another organism, or it can be, for example, an mRNA molecule that is not normally expressed in a cell or sample.

As used herein, a “host cell” is a cell that is used to receive, maintain, reproduce and amplify an expression vector. A host cell also can be used to express the polypeptide encoded by the expression vector. The nucleic acid contained in the expression vector is replicated when the host cell divides, thereby amplifying the nucleic acids.

As used herein, the term “immune cell” refers to any cell that plays a role in the immune response of a subject. Immune cells are of hematopoietic origin, and include lymphocytes, such as B cells and T cells; natural killer cells; myeloid cells, such as monocytes, macrophages, dendritic cells, eosinophils, neutrophils, mast cells, basophils, and granulocytes. As used herein, the term “engineered immune cell” refers to an immune cell that is genetically modified. As used herein, the term “native immune cell” refers to an immune cell that naturally occurs in the immune system.

As used herein, the term “increase” or “enhance” means to alter positively by at least about 5%, including, but not limited to, alter positively by about 5%, by about 10%, by about 25%, by about 30%, by about 50%, by about 75%, or by about 100%.

As used herein, the term “isolated,” “purified,” or “biologically pure” refers to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or polypeptide of the presently disclosed subject matter is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

As used herein, the term “isolated cell” refers to a cell that is separated from the molecular and/or cellular components that naturally accompany the cell.

As used herein, the term “ligand” refers to a molecule that binds to a receptor. In particular, the ligand binds a receptor on another cell, allowing for cell-to-cell recognition and/or interaction.

The term “linker” refers to synthetic sequences (e.g., amino acid sequences) that connect or link two sequences, e.g., that link two polypeptide domains. In some embodiments, the linker comprises from a total of 1 to 200 amino acid residues; or about 1 to 10 amino acid residues, or alternatively 8 amino acids, or alternatively 6 amino acids, or alternatively 5 amino acids that may be repeated from 1 to 10, or alternatively to about 8, or alternatively to about 6, or alternatively to about 5, or alternatively, to about 4, or alternatively to about 3, or alternatively to about 2 times. For example, the linker may comprise up to 15 amino acid residues consisting of a pentapeptide repeated three times. In one embodiment, the linker sequence is a (G4S)n (SEQ ID NO: 16), wherein n is 1, or 2, or 3, or 4, or 5, or 6, or 7, or 8, or 9, or 10, or 11, or 12, or 13, or 14, or 15.

The term “lymphocyte” refers to all immature, mature, undifferentiated, and differentiated white blood cell populations that are derived from lymphoid progenitors including tissue specific and specialized varieties, and encompasses, by way of non-limiting example, B cells, T cells, NKT cells, and NK cells. In some embodiments, lymphocytes include all B cell lineages including pre-B cells, progenitor B cells, early pro-B cells, late pro-B cells, large pre-B cells, small pre-B cells, immature B cells, mature B cells, plasma B cells, memory B cells, B-1 cells, B-2 cells, and anergic AN1/T3 cell populations.

As used herein, “operably linked” with reference to nucleic acid sequences, regions, elements or domains means that the nucleic acid regions are functionally related to each other. For example, a nucleic acid encoding a leader peptide can be operably linked to a nucleic acid encoding a polypeptide, whereby the nucleic acids can be transcribed and translated to express a functional fusion protein, wherein the leader peptide affects secretion of the fusion polypeptide. In some instances, the nucleic acid encoding a first polypeptide (e.g., a leader peptide) is operably linked to nucleic acid encoding a second polypeptide and the nucleic acids are transcribed as a single mRNA transcript, but translation of the mRNA transcript can result in one of two polypeptides being expressed. For example, an amber stop codon can be located between the nucleic acid encoding the first polypeptide and the nucleic acid encoding the second polypeptide, such that, when introduced into a partial amber suppressor cell, the resulting single mRNA transcript can be translated to produce either a fusion protein containing the first and second polypeptides, or can be translated to produce only the first polypeptide. In another example, a promoter can be operably linked to nucleic acid encoding a polypeptide, whereby the promoter regulates or mediates the transcription of the nucleic acid.

“Optional” or “optionally” means that the subsequently described circumstance may or may not occur, so that the description includes instances where the circumstance occurs and instances where it does not.

As used herein, the “percent homology” between two amino acid sequences is equivalent to the percent identity between the two sequences. The percent identity between the two sequences is a function of the number of identical positions shared by the sequences (i.e., % homology=#of identical positions/total #of positions×100), taking into account the number of gaps, and the length of each gap, which need to be introduced for optimal alignment of the two sequences. The comparison of sequences and determination of percent identity between two sequences can be accomplished using a mathematical algorithm.

The percent homology between two amino acid sequences can be determined using the algorithm of E. Meyers and W. Miller (Comput. Appl. Biosci., 4:1 1-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the amino acids sequences of the presently disclosed subject matter can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the specified sequences disclosed herein. To obtain gapped alignments for comparison purposes, Gapped BLAST can be utilized as described in Altschul et al., (1997) Nucleic Acids Res. 25 (17): 3389-3402. When utilizing BLAST and Gapped BLAST programs, the default parameters of the respective programs (e.g., XBLAST and NBLAST) can be used.

“Pharmaceutically acceptable carriers” refers to any diluents, excipients, or carriers that may be used in the compositions disclosed herein. In some embodiments, a pharmaceutically acceptable carrier comprises, or consists essentially of, or yet further consists of a nanoparticle, such as an polymeric nanoparticle carrier or an lipid nanoparticle (LNP). Additionally or alternatively, pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They can be selected with respect to the intended form of administration, that is, oral tablets, capsules, elixirs, syrups and the like, and consistent with conventional pharmaceutical practices.

The terms “polynucleotide”, “nucleic acid” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this disclosure that is a polynucleotide encompasses both the double-stranded form and each of two complementary single-stranded forms known or predicted to make up the double-stranded form. A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

The terms “polypeptide,” “peptide,” and “protein” are used interchangeably herein to refer to a polymer of amino acid residues. The terms apply to naturally occurring amino acid polymers as well as amino acid polymers in which one or more amino acid residues are a non-naturally occurring amino acid, e.g., an amino acid analog. The terms encompass amino acid chains of any length, including full length proteins, wherein the amino acid residues are linked by covalent peptide bonds.

As used herein, the term “reduce” means to alter negatively by at least about 5%, including, but not limited to, alter negatively by about 5%, by about 10%, by about 25%, by about 30%, by about 50%, by about 75%, or by about 100%.

As used herein, “regulatory sequence” of a nucleic acid molecule means a cis-acting nucleotide sequence that influences expression, positively or negatively, of an operably linked gene. Regulatory regions include sequences of nucleotides that confer inducible (i.e., require a substance or stimulus for increased transcription) expression of a gene. When an inducer is present or at increased concentration, gene expression can be increased. Regulatory regions also include sequences that confer repression of gene expression (i.e., a substance or stimulus decreases transcription). When a repressor is present or at increased concentration, gene expression can be decreased. Regulatory regions are known to influence, modulate or control many in vivo biological activities including cell proliferation, cell growth and death, cell differentiation and immune modulation. Regulatory regions typically bind to one or more trans-acting proteins, which results in either increased or decreased transcription of the gene.

Particular examples of gene regulatory regions are promoters and enhancers. Promoters are sequences located around the transcription or translation start site, typically positioned 5′ of the translation start site. Promoters usually are located within 1 Kb of the translation start site, but can be located further away, for example, 2 Kb, 3 Kb, 4 Kb, 5 Kb or more, up to and including 10 Kb. Polymerase II and III are examples of promoters. A polymerase II or “pol II” promoter catalyzes the transcription of DNA to synthesize precursors of mRNA, and most shRNA and microRNA. Examples of pol II promoters are known in the art and include without limitation, the phosphoglycerate kinase (“PGK”) promoter; EF1-alpha; CMV (minimal cytomegalovirus promoter); and LTRs from retroviral and lentiviral vectors. In some embodiments, the promoter is a constitutive promoter. As used herein, the term “constitutive promoter” refers to a promoter that allows for continual transcription of the coding sequence or gene under its control in all or most tissues of a subject at all or most developing stages. Non-limiting examples of the constitutive promoters include a CMV promoter, a simian virus 40 (SV40) promoter, a polyubiquitin C (UBC) promoter, an EF1-alpha promoter, a PGK promoter and a CAG promoter. In some embodiments, the promoter is a conditional promoter, which allows for continual transcription of the coding sequence or gene under certain conditions. In further embodiments, the conditional promoter is an immune cell specific promoter, which allows for continual transcription of the coding sequence or gene in an immune cell. Non-limiting examples of the immune cell specific promoters include a promoter of a B29 gene promoter, a CD14 gene promoter, a CD43 gene promoter, a CD45 gene promoter, a CD68 gene promoter, a IFN-β gene promoter, a WASP gene promoter, a T-cell receptor β-chain gene promoter, a V9γ (TRGV9) gene promoter, a V2δ (TRDV2) gene promoter, and the like.

Enhancers are known to influence gene expression when positioned 5′ or 3′ of the gene, or when positioned in or a part of an exon or an intron. Enhancers also can function at a significant distance from the gene, for example, at a distance from about 3 Kb, 5 Kb, 7 Kb, 10 Kb, 15 Kb or more.

Regulatory regions also include, but are not limited to, in addition to promoter regions, sequences that facilitate translation, splicing signals for introns, maintenance of the correct reading frame of the gene to permit in-frame translation of mRNA and, stop codons, leader sequences and fusion partner sequences, internal ribosome binding site (IRES) elements for the creation of multigene, or polycistronic, messages, polyadenylation signals to provide proper polyadenylation of the transcript of a gene of interest and stop codons, and can be optionally included in an expression vector.

As used herein, the term “sample” refers to clinical samples obtained from a subject. In certain embodiments, a sample is obtained from a biological source (i.e., a “biological sample”), such as tissue, bodily fluid, or microorganisms collected from a subject. Sample sources include, but are not limited to, mucus, sputum, bronchial alveolar lavage (BAL), bronchial wash (BW), whole blood, bodily fluids, cerebrospinal fluid (CSF), urine, plasma, serum, or tissue.

As used herein, the term “secreted” in reference to a polypeptide means a polypeptide that is released from a cell via the secretory pathway through the endoplasmic reticulum, Golgi apparatus, and as a vesicle that transiently fuses at the cell plasma membrane, releasing the proteins outside of the cell. Small molecules, such as drugs, can also be secreted by diffusion through the membrane to the outside of cell.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

As used herein, the term “specifically binds” or “specifically binds to” or “specifically target” refers to a molecule (e.g., a polypeptide or fragment thereof) that recognizes and binds a molecule of interest (e.g., an antigen), but which does not substantially recognize and bind other molecules. The terms “specific binding,” “specifically binds to,” or is “specific for” a particular molecule (e.g., an antigen), as used herein, can be exhibited, for example, by a molecule having a K_{d} for the molecule to which it binds to of about 10⁻⁴M, 10⁻⁵M, 10⁻⁶M, 10⁻⁷M, 10⁻⁸M, 10⁻⁹M, 10⁻¹⁰M, 10⁻¹¹ M, or 10⁻¹² M.

As used herein, the terms “subject,” “individual,” or “patient” are used interchangeably and refer to an individual organism, a vertebrate, or a mammal and may include humans, non-human primates, rodents, and the like (e.g., which is to be the recipient of a particular treatment, or from whom cells are harvested). In certain embodiments, the individual, patient or subject is a human.

“Substantially” or “essentially” means nearly totally or completely, for instance, 95% or greater of some given quantity. In some embodiments, “substantially” or “essentially” means 95%, 96%, 97%, 98%, 99%, 99.5%, or 99.9%.

The terms “substantially homologous” or “substantially identical” mean a polypeptide or nucleic acid molecule that exhibits at least 50% or greater homology or identity to a reference amino acid sequence (for example, any one of the amino acid sequences described herein) or nucleic acid sequence (for example, any one of the nucleic acid sequences described herein). For example, such a sequence is at least about 60%, about 65%, about 70%, about 75%, about 80%, about 85%, about 90%, about 95% or about 99% homologous or identical at the amino acid level or nucleic acid to the sequence used for comparison (e.g., a wild-type, or native, sequence). In some embodiments, a substantially homologous or substantially identical polypeptide contains one or more amino acid substitutions, insertions, or deletions relative to the sequence used for comparison. In some embodiments, a substantially homologous or substantially identical polypeptide contains one or more non-natural amino acids or amino acid analogs, including, D-amino acids and retroinverso amino, to replace homologous sequences.

Sequence homology or sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. In an exemplary approach to determining the degree of identity, a BLAST program may be used, with a probability score between e⁻³ and e⁻¹⁰⁰ indicating a closely related sequence.

Nucleic acid molecules useful in the presently disclosed subject matter include any nucleic acid molecule that encodes a polypeptide or a fragment thereof. In certain embodiments, nucleic acid molecules useful in the presently disclosed subject matter include nucleic acid molecules that encode an antibody or an antigen binding portion thereof. Such nucleic acid molecules need not be 100% identical with an endogenous nucleic acid sequence, but will typically exhibit substantial identity. Polynucleotides having “substantial homology” or “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger, Methods Enzymol. 152:399 (1987); Kimmel, A. R. Methods Enzymol. 152:507 (1987)). For example, stringent salt concentration will ordinarily be less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% w/v formamide, or at least about 50% w/v formamide. Stringent temperature conditions will ordinarily include temperatures of at least about 30° C., at least about 37° C., or at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In certain embodiments, hybridization will occur at 30° C. in 750 mM NaCl, 75 mM trisodium citrate, and 1% w/v SDS. In certain embodiments, hybridization will occur at 37° C. in 500 mM NaCl, 50 mM trisodium citrate, 1% w/v SDS, 35% w/v formamide, and 100 μg/ml denatured salmon sperm DNA (ssDNA). In certain embodiments, hybridization will occur at 42° C. in 250 mM NaCl, 25 mM trisodium citrate, 1% w/v SDS, 50% w/v formamide, and 200 μg ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will less than about 30 mM NaCl and 3 mM trisodium citrate, or less than about 15 mM NaCl and 1.5 mM trisodium citrate. Stringent temperature conditions for the wash steps will ordinarily include a temperature of at least about 25° C., at least about 42° C., or at least about 68° C. In certain embodiments, wash steps will occur at 25° C. in 30 mM NaCl, 3 mM trisodium citrate, and 0.1% w/v SDS. In certain embodiments, wash steps will occur at 42° C. in 15 mM NaCl, 1.5 mM trisodium citrate, and 0.1% w/v SDS. In certain embodiments, wash steps will occur at 68° C. in 15 mM NaCl, 1.5 mM trisodium citrate, and 0.1% w/v SDS. Additional variations on these conditions will be readily apparent to those skilled in the art. Hybridization techniques are well known to those skilled in the art and are described, for example, in Benton and Davis (Science 196:180 (1977)); Grunstein and Rogness (Proc. Natl. Acad. Sci., USA 72:3961 (1975)); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

As used herein, “synthetic,” with reference to, for example, a synthetic nucleic acid molecule or a synthetic gene or a synthetic peptide refers to a nucleic acid molecule or polypeptide molecule that is produced by recombinant methods and/or by chemical synthesis methods. As used herein, production by recombinant means by using recombinant DNA methods means the use of the well-known methods of molecular biology for expressing proteins encoded by cloned DNA.

As used herein, the term “T-cell” includes naïve T cells, CD4⁺ T cells, CD8⁺ T cells, memory T cells (including central memory T cells, stem-cell-like memory T cells (or stem-like memory T cells), and two types of effector memory T cells: e.g., TEM cells and TEMRA cells), activated T cells, anergic T cells, tolerant T cells, chimeric B cells, Regulatory T cells (also known as suppressor T cells), Natural killer T cells, Mucosal associated invariant T cells, and γδ T cells, and antigen-specific T cells.

As used herein, “T cell receptor” or “TCR”, is a protein complex found on the surface of T cells, that is responsible for recognizing fragments of antigen as peptides bound to major histocompatibility complex molecules. TCR is composed of two disulfide-linked protein chains. Cells expressing a TCR containing the highly variable alpha (α) and beta (β) chains are referred to as αβ T cells. Cells expressing an alternate TCR, formed by variable gamma (γ) and delta (δ) chains, are referred to as γδ T cells. When the TCR engages with antigenic peptide and MHC (peptide/MHC), the T lymphocyte is activated through signal transduction, that is, a series of biochemical events mediated by associated enzymes, co-receptors, specialized adaptor molecules, and activated or released transcription factors. In some embodiments, the TCR is a native T cell receptor that is endogenous to the immune cells. In some embodiments, the TCR is an artificial receptor that mimics native TCR function, i.e., recognizing peptide antigens of key intracellular proteins in the context of MHC on the cell surface.

As used herein “tumor-infiltrating immune cells” or “TILs” refer to immune cells that have left the bloodstream and migrated into a tumor.

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. Therapeutic effects of treatment include, without limitation, inhibiting recurrence of disease, alleviation of symptoms, diminishment of any direct or indirect pathological consequences of the disease, preventing metastases, decreasing the rate of disease progression, amelioration or palliation of the disease state, and remission or improved prognosis. By “treating a cancer” is meant that the symptoms associated with the cancer are, e.g., alleviated, reduced, cured, or placed in a state of remission.

It is also to be appreciated that the various modes of treatment of diseases as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

The compositions used in accordance with the disclosure can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions are administered in a manner compatible with the dosage formulation and in such amount as is therapeutically or prophylactically effective. The formulations are easily administered in a variety of dosage forms, such as the type of injectable solutions described herein.

### Adoptive Cell Therapy (ACT)

CAR T cell therapy has gained momentum after several promising clinical trials for the treatment of B-cell neoplasms and the FDA approval of a CD19 targeted CAR T cell for treatment of B cell acute lymphoid leukemia (Sadelain et al., Nature 545:423-431 (2017); Yu et al., J Hematol Oncol. 10:78 (2017); Kakarla and Gottschalk, Cancer J. 20:151-155 (2014); Wang et al., J Hematol Oncol. 10:53 (2017)). CAR T cell therapy involves isolating a patient's own T cells, engineering them to express a CAR, and reinfusing the engineered T cells back into the patient. The CAR contains an extracellular single-chain variable fragment (scFv), a transmembrane domain, and an intracellular signaling domain. Surface expression of a tumor-targeted scFv on the T cell results in tumor antigen-directed T cell activation and specific tumor killing via its signaling domain. However, many patients with hematologic cancers treated with CAR T cell therapy relapse with antigen loss variants as a result of tumor editing (Wang et al., J Hematol Oncol. 10:53 (2017)). Furthermore, translation of CAR T cell therapy to solid tumors has been difficult due to the immunosuppressive tumor environment (TME) (Yu et al., J Hematol Oncol. 10:78 (2017); Kakarla and Gottschalk, Cancer J. 20:151-155 (2014)).

In some embodiments, the engineered immune cells provided herein express a receptor or other cell-surface ligand that binds to a U5 snRNP200 antigen. In some embodiments, the receptor is a wild-type or native receptor. In some embodiments, the receptor is an engineered receptor or a non-native receptor. In some embodiments, the engineered receptor is an engineered TCR (eTCR). In some embodiments, the engineered receptor is a chimeric antibody TCR (caTCR). In some embodiments, the engineered receptor is a chimeric antigen receptor (CAR).

In exemplary embodiments, the engineered immune cells provided herein express a native receptor, a non-native receptor, or an engineered receptor (e.g., a CAR, caTCR, or eTCR) or other cell-surface ligand that binds to a U5 snRNP200 antigen.

### Chimeric Antigen Receptors

In some embodiments, the engineered immune cells provided herein express at least one chimeric antigen receptor (CAR). CARs are engineered receptors, which graft or confer a specificity of interest onto an immune effector cell. For example, CARs can be used to graft the specificity of a monoclonal antibody onto an immune cell, such as a T cell. In some embodiments, transfer of the coding sequence of the CAR is facilitated by nucleic acid vector, such as a retroviral vector.

There are currently three generations of CARs. In some embodiments, the engineered immune cells provided herein express a “first generation” CAR. “First generation” CARs are typically composed of an extracellular antigen binding domain (e.g., a single-chain variable fragment (scFv)) fused to a transmembrane domain fused to cytoplasmic/intracellular domain of the T cell receptor (TCR) chain. “First generation” CARs typically have the intracellular domain from the CD3ζ chain, which is the primary transmitter of signals from endogenous TCRs. “First generation” CARs can provide de novo antigen recognition and cause activation of both CD4⁺ and CD8⁺ T cells through their CD3ζ chain signaling domain in a single fusion molecule, independent of HLA-mediated antigen presentation.

In some embodiments, the engineered immune cells provided herein express a “second generation” CAR. “Second generation” CARs add intracellular domains from various co-stimulatory molecules (e.g., CD28, 4-1BB, ICOS, OX40) to the cytoplasmic tail of the CAR to provide additional signals to the T cell. “Second generation” CARs comprise those that provide both co-stimulation (e.g., CD28 or 4-1BB) and activation (e.g., CD35). Preclinical studies have indicated that “Second Generation” CARs can improve the antitumor activity of T cells. For example, robust efficacy of “Second Generation” CAR modified T cells was demonstrated in clinical trials targeting the CD19 molecule in patients with chronic lymphoblastic leukemia (CLL) and acute lymphoblastic leukemia (ALL).

In some embodiments, the engineered immune cells provided herein express a “third generation” CAR. “Third generation” CARs comprise those that provide multiple co-stimulation (e.g., CD28 and 4-1BB) and activation (e.g., CD3).

In accordance with the presently disclosed subject matter, the CARs of the engineered immune cells provided herein comprise an extracellular antigen-binding domain, a transmembrane domain and an intracellular domain. Further, the activity of the engineered immune cells can be adjusted by selection of co-stimulatory molecules included in the chimeric antigen receptor.

Extracellular Antigen-Binding Domain of a CAR. In certain embodiments, the extracellular antigen-binding domain of a CAR specifically binds a U5 snRNP200 antigen. In certain embodiments, the extracellular antigen-binding domain is derived from a monoclonal antibody (mAb) that binds to a U5 snRNP200 antigen. In some embodiments, the extracellular antigen-binding domain comprises an scFv. In some embodiments, the extracellular antigen-binding domain comprises a Fab, which is optionally crosslinked. In some embodiments, the extracellular binding domain comprises a F(ab)₂. In some embodiments, any of the foregoing molecules are included in a fusion protein with a heterologous sequence to form the extracellular antigen-binding domain. In certain embodiments, the extracellular antigen-binding domain comprises a human scFv that binds specifically to a U5 snRNP200 antigen. In certain embodiments, the scFv is identified by screening scFv phage library with a U5 snRNP200 antigen-Fc fusion protein.

In certain embodiments, the extracellular antigen-binding domain of a presently disclosed CAR has a high binding specificity and high binding affinity to a U5 snRNP200 antigen. For example, in some embodiments, the extracellular antigen-binding domain of the CAR (embodied, for example, in a human scFv or an analog thereof) binds to a particular U5 snRNP200 antigen with a dissociation constant (K_{d}) of about 1×10⁻⁵ M or less. In certain embodiments, the K_{d} is about 5×10⁻⁶ M or less, about 1×10⁻⁶ M or less, about 5×10⁻⁷ M or less, about 1×10⁻⁷ M or less, about 5×10⁻⁸ M or less, about 1×10⁻⁸ M or less, about 5×10⁻⁹ or less, about 4×10⁻⁹ or less, about 3×10⁻⁹ or less, about 2×10⁻⁹ or less, or about 1×10⁻⁹ M or less. In certain non-limiting embodiments, the K_{d} is from about 3×10⁻⁹ M or less. In certain non-limiting embodiments, the K_{d} is from about 3×10⁻⁹ to about 2×10⁻⁷.

Binding of the extracellular antigen-binding domain (embodiment, for example, in an scFv or an analog thereof) of a presently disclosed U5 snRNP200-specific CAR can be confirmed by, for example, enzyme-linked immunosorbent assay (ELISA), radioimmunoassay (RIA), FACS analysis, bioassay (e.g., growth inhibition), or Western Blot assay. Each of these assays generally detect the presence of protein-antibody complexes of particular interest by employing a labeled reagent (e.g., an antibody, or an scFv) specific for the complex of interest. For example, the scFv can be radioactively labeled and used in a radioimmunoassay (RIA) (see, for example, Weintraub, B., Principles of Radioimmunoassays, Seventh Training Course on Radioligand Assay Techniques, The Endocrine Society, March 1986, which is incorporated by reference herein). The radioactive isotope can be detected by such means as the use of a γ counter or a scintillation counter or by autoradiography. In certain embodiments, the extracellular antigen-binding domain of the U5 snRNP200-specific CAR is labeled with a fluorescent marker. Non-limiting examples of fluorescent markers include green fluorescent protein (GFP), blue fluorescent protein (e.g., EBFP, EBFP2, Azurite, and mKalamal), cyan fluorescent protein (e.g., ECFP, Cerulean, and CyPet), and yellow fluorescent protein (e.g., YFP, Citrine, Venus, and YPet). In certain embodiments, the scFv of a presently disclosed U5 snRNP200-specific CAR is labeled with GFP.

In some embodiments, the extracellular antigen-binding domain of the expressed CAR binds to a U5 snRNP200 antigen that is expressed by a tumor cell. In some embodiments, the extracellular antigen-binding domain of the expressed CAR binds to a U5 snRNP200 antigen that is expressed on the surface of a tumor cell.

In certain embodiments, the extracellular antigen-binding domain (e.g., human scFv) comprises a heavy chain variable (V_{H}) region and a light chain variable (V_{L}) region, optionally linked with a linker sequence, for example a linker peptide (e.g., SEQ ID NO: 3), between the heavy chain variable (V_{H}) region and the light chain variable (V_{L}) region.

In certain non-limiting embodiments, an extracellular antigen-binding domain of the presently disclosed CAR can comprise a linker connecting the heavy chain variable (V_{H}) region and light chain variable (V_{L}) region of the extracellular antigen-binding domain. As used herein, the term “linker” refers to a functional group (e.g., chemical or polypeptide) that covalently attaches two or more polypeptides or nucleic acids so that they are connected to one another. As used herein, a “peptide linker” refers to one or more amino acids used to couple two proteins together (e.g., to couple V_{H} and V_{L} domains). In certain embodiments, the linker comprises amino acids having the sequence set forth in SEQ ID NO: 3. In certain embodiments, the nucleotide sequence encoding the amino acid sequence of SEQ ID NO: 3 is set forth in SEQ ID NO: 4.

Additionally or alternatively, in some embodiments, the extracellular antigen-binding domain can comprise a leader or a signal peptide sequence that directs the nascent protein into the endoplasmic reticulum. The signal peptide or leader can be essential if the CAR is to be glycosylated and anchored in the cell membrane. The signal sequence or leader sequence can be a peptide sequence (about 5, about 10, about 15, about 20, about 25, or about 30 amino acids long) present at the N-terminus of the newly synthesized proteins that direct their entry to the secretory pathway.

In certain embodiments, the signal peptide is operably linked to the N-terminus of the extracellular antigen-binding domain. In certain embodiments, the signal peptide comprises a human CD8 signal polypeptide comprising amino acids having the sequence set forth in SEQ ID NO: 41 as provided below: MALPVTALLLPLALLLHAARP (SEQ ID NO: 41).

The nucleotide sequence encoding the amino acid sequence of SEQ ID NO: 41 is set forth in SEQ ID NO: 42, which is provided below:

(SEQ ID NO: 42)
ATGGCCCTGCCAGTAACGGCTCTGCTGCTGCCACTTGCTCTGCT
CCTCCATGCAGCCAGGCCT.

In certain embodiments, the signal peptide comprises a human CD8 signal polypeptide comprising amino acids having the sequence set forth in SEQ ID NO: 43 as provided below: MALPVTALLLPLALLLHA (SEQ ID NO: 43).

The nucleotide sequence encoding the amino acid sequence of SEQ ID NO: 43 is set forth in SEQ ID NO: 44, which is provided below:

(SEQ ID NO: 44)
ATGGCTCTCCCAGTGACTGCCCTACTGCTTCCCCTAGCGCTTCT
CCTGCATGCA.

In certain embodiments, the signal peptide comprises a mouse CD8 signal polypeptide comprising amino acids having the sequence set forth in SEQ ID NO: 45 as provided below: MASPLTRFLSLNLLLLGESII (SEQ ID NO: 45).

The nucleotide sequence encoding the amino acid sequence of SEQ ID NO: 45 is set forth in SEQ ID NO: 46, which is provided below:

(SEQ ID NO: 46)
ATGGCCAGCCCCCTGACCAGGTTCCTGAGCCTGAACCTGCTGCT
GCTGGGCGAGAGCATCATC.

In certain embodiments, the signal peptide comprises a mouse CD8 signal polypeptide comprising amino acids having the sequence set forth in SEQ ID NO: 47 as provided below: MASPLTRFLSLNLLLLGE (SEQ ID NO: 47).

The nucleotide sequence encoding the amino acid sequence of SEQ ID NO: 47 is set forth in SEQ ID NO: 48, which is provided below:

(SEQ ID NO: 48)
ATGGCCAGCCCCCTGACCAGGTTCCTGAGCCTGAACCTGCTGCT
GCTGGGCGAG.

Transmembrane Domain of a CAR. In certain non-limiting embodiments, the transmembrane domain of the CAR comprises a hydrophobic alpha helix that spans at least a portion of the membrane. Different transmembrane domains result in different receptor stability. After antigen recognition, receptors cluster and a signal is transmitted to the cell. In accordance with the presently disclosed subject matter, the transmembrane domain of the CAR can comprise a CD8 polypeptide, a CD28 polypeptide, a CD3ζ polypeptide, a CD4 polypeptide, a 4-1BB polypeptide, an OX40 polypeptide, an ICOS polypeptide, a CTLA-4 polypeptide, a PD-1 polypeptide, a LAG-3 polypeptide, a 2B4 polypeptide, a BTLA polypeptide, a synthetic peptide (e.g., a transmembrane peptide not based on a protein associated with the immune response), or a combination thereof.

In certain embodiments, the transmembrane domain of a presently disclosed CAR comprises a CD28 polypeptide. The CD28 polypeptide can have an amino acid sequence that is at least about 85%, about 90%, about 95%, about 96%, about 97%, about 98%, about 99% or 100% homologous to the sequence having a UniProtKB Reference No: P10747 or NCBI Reference No: NP006130 (SEQ ID NO: 49), or fragments thereof, and/or may optionally comprise up to one or up to two or up to three conservative amino acid substitutions. In certain embodiments, the CD28 polypeptide can have an amino acid sequence that is a consecutive portion of SEQ ID NO: 49 which is at least 20, or at least 30, or at least 40, or at least 50, and up to 220 amino acids in length. Additionally or alternatively, in non-limiting various embodiments, the CD28 polypeptide has an amino acid sequence of amino acids 1 to 220, 1 to 50, 50 to 100, 100 to 150, 114 to 220, 150 to 200, or 200 to 220 of SEQ ID NO: 49. In certain embodiments, the CAR of the present disclosure comprises a transmembrane domain comprising a CD28 polypeptide, and optionally an intracellular domain comprising a co-stimulatory signaling region that comprises a CD28 polypeptide. In certain embodiments, the CD28 polypeptide comprised in the transmembrane domain and the intracellular domain has an amino acid sequence of amino acids 114 to 220 of SEQ ID NO: 49. In certain embodiments, the CD28 polypeptide comprised in the transmembrane domain has an amino acid sequence of amino acids 153 to 179 of SEQ ID NO: 49.

SEQ ID NO: 49 is provided below:

(SEQ ID NO: 49)
MLRLLLALNLFPSIQVTGNKILVKQSPMLVAYDNAVNLSCKYSY
NLFSREFRASLHKGLDSAVEVCVVYGNYSQQLQVYSKTGFNCDG
KLGNESVTFYLQNLYVNQTDIYFCKIEVMYPPPYLDNEKSNGTI
IHVKGKHLCPSPLFPGPSKPFWVLVVVGGVLACYSLLVTVAFII
FWVRSKRSRLLHSDYMNMTPRRPGPTRKHYQPYAPPRDFAAYRS

In accordance with the presently disclosed subject matter, a “CD28 nucleic acid molecule” refers to a polynucleotide encoding a CD28 polypeptide. In certain embodiments, the CD28 nucleic acid molecule encoding the CD28 polypeptide comprised in the transmembrane domain (and optionally the intracellular domain (e.g., the co-stimulatory signaling region)) of the presently disclosed CAR (e.g., amino acids 114 to 220 of SEQ ID NO: 49 or amino acids 153 to 179 of SEQ ID NO: 49) comprises at least a portion of the sequence set forth in SEQ ID NO: 50 as provided below.

(SEQ ID NO: 50)
attgaagttatgtatcctcctccttacctagacaatgagaagag
caatggaaccattatccatgtgaaagggaaacacctttgtccaa
gtcccctatttcccggaccttctaagcccttttgggtgctggtg
gtggttggtggagtcctggcttgctatagcttgctagtaacagt
ggcctttattattttctgggtgaggagtaagaggagcaggctcc
tgcacagtgactacatgaacatgactccccgccgccccgggccc
acccgcaagcattaccagccctatgccccaccacgcgacttcgc
agcctatcgctcc

In some embodiments, the CD28 polypeptide comprised in the transmembrane domain and the intracellular domain has an amino acid sequence of

(SEQ ID NO: 74)
AAAIEVMYPPPYLDNEKSNGTIIHVKGKHLCPSPLFPGPSKPFW
VLVVVGGVLACYSLLVTVAFIIFWVRSKRSRLLHSDYMNMTPRR
PGPTRKHYQPYAPPRDFAAYRS;
or
(SEQ ID NO: 83)
AAAIEVMYPPPYLDNEKSNGTIIHVKGKHLCPSPLFPGPSKPFW
VLVVVGGVLACYSLLVTVAFIIFWVRSKRSRLLHSDYSNVTPRR
PGPTRKHYQPYAPPRDFAAYRS

In certain embodiments, the transmembrane domain comprises a CD8 polypeptide. The CD8 polypeptide can have an amino acid sequence that is at least about 85%, about 90%, about 95%, about 96%, about 97%, about 98%, about 99% or about 100%) homologous to SEQ ID NO: 51 (homology herein may be determined using standard software such as BLAST or FASTA) as provided below, or fragments thereof, and/or may optionally comprise up to one or up to two or up to three conservative amino acid substitutions. In certain embodiments, the CD8 polypeptide can have an amino acid sequence that is a consecutive portion of SEQ ID NO: 51 which is at least 20, or at least 30, or at least 40, or at least 50, and up to 235 amino acids in length. Additionally or alternatively, in various embodiments, the CD8 polypeptide has an amino acid sequence of amino acids 1 to 235, 1 to 50, 50 to 100, 100 to 150, 150 to 200, or 200 to 235 of SEQ ID NO: 51.

(SEQ ID NO: 51)
MALPVTALLLPLALLLHAARPSQFRVSPLDRTWNLGETVELKCQ
VLLSNPTSGCSWLFQPRGAAASPTFLLYLSQNKPKAAEGLDTQR
FSGKRLGDTFVLTLSDFRRENEGYYFCSALSNSIMYFSHFVPVF
LPAKPTTTPAPRPPTPAPTIASQPLSLRPEACRPAAGGAVHTRG
LDFACDIYIWAPLAGTCGVLLLSLVITLYCNHRNRRRVCKCPRP
WKSGDKPSLSARYV

In certain embodiments, the transmembrane domain comprises a CD8 polypeptide comprising amino acids having the sequence set forth in SEQ ID NO: 52 as provided below:

(SEQ ID NO: 52)
PTTTPAPRPPTPAPTIASQPLSLRPEACRPAAGGAVHTRGLDFA
CDIYIWAPLAGTCGVLLLSLVITLYCN

In accordance with the presently disclosed subject matter, a “CD8 nucleic acid molecule” refers to a polynucleotide encoding a CD8 polypeptide. In certain embodiments, the CD8 nucleic acid molecule encoding the CD8 polypeptide comprised in the transmembrane domain of the presently disclosed CAR (SEQ ID NO: 52) comprises nucleic acids having the sequence set forth in SEQ ID NO: 53 as provided below.

(SEQ ID NO: 53)
CCCACCACGACGCCAGCGCCGCGACCACCAACCCCGGCGCCCAC
GATCGCGTCGCAGCCCCTGTCCCTGCGCCCAGAGGCGTGCCGGC
CAGCGGCGGGGGGCGCAGTGCACACGAGGGGGCTGGACTTCGCC
TGTGATATCTACATCTGGGCGCCCCTGGCCGGGACTTGTGGGGT
CCTTCTCCTGTCACTGGTTATCACCCTTTACTGCAAC

In certain non-limiting embodiments, a CAR can also comprise a spacer region that links the extracellular antigen-binding domain to the transmembrane domain. The spacer region can be flexible enough to allow the antigen-binding domain to orient in different directions to facilitate antigen recognition while preserving the activating activity of the CAR. In certain non-limiting embodiments, the spacer region can be the hinge region from IgG1, the C_{H}2C_{H}3 region of immunoglobulin and portions of CD3, a portion of a CD28 polypeptide (e.g., SEQ ID NO: 49), a portion of a CD8 polypeptide (e.g., SEQ ID NO: 51), a variation of any of the foregoing which is at least about 80%, at least about 85%, at least about 90%, or at least about 95% homologous thereto, or a synthetic spacer sequence. In certain non-limiting embodiments, the spacer region may have a length between about 1-50 (e.g., 5-25, 10-30, or 30-50) amino acids.

Intracellular Domain of a CAR. In certain non-limiting embodiments, an intracellular domain of the CAR can comprise a CD35 polypeptide, which can activate or stimulate a cell (e.g., a cell of the lymphoid lineage, e.g., a T cell). CD3ζ comprises 3 ITAMs, and transmits an activation signal to the cell (e.g., a cell of the lymphoid lineage, e.g., a T cell) after antigen is bound. The CD3 polypeptide can have an amino acid sequence that is at least about 85%, about 90%, about 95%, about 96%, about 97%, about 98%, about 99% or about 100% homologous to the sequence having a NCBI Reference No: NP_932170 (SEQ ID NO: 54), or fragments thereof, and/or may optionally comprise up to one or up to two or up to three conservative amino acid substitutions.

In certain embodiments, the CD3ζ polypeptide can have an amino acid sequence that is a consecutive portion of SEQ ID NO: 55 which is at least 20, or at least 30, or at least 40, or at least 50, and up to 164 amino acids in length. Additionally or alternatively, in various embodiments, the CD3ζ polypeptide has an amino acid sequence of amino acids 1 to 164, 1 to 50, 50 to 100, 100 to 150, or 150 to 164 of SEQ ID NO: 55. In certain embodiments, the CD3ζ polypeptide has an amino acid sequence of amino acids 52 to 164 of SEQ ID NO: 55.

SEQ ID NO: 55 is provided below:

(SEQ ID NO: 55)
MKWKALFTAAILQAQLPITEAQSFGLLDPKLCYLLDGILFIYGV
ILTALFLRVKFSRSADAPAYQQGQNQLYNELNLGRREEYDVLDK
RRGRDPEMGGKPQRRKNPQEGLYNELQKDKMAEAYSEIGMKGER
RRGKGHDGLYQGLSTATKDTYDALHMQALPPR

In certain embodiments, the CD3ζ polypeptide has the amino acid sequence set forth in SEQ ID NO: 56, which is provided below:

(SEQ ID NO: 56)
RVKFSRSAEPPAYQQGQNQLYNELNLGRREEYDVLDKRRGRDPE
MGGKPRRKNPQEGLYNELQKDKMAEAYSEIGMKGERRRGKGHDG
LYQGLSTATKDTYDALHMQALPPR

In certain embodiments, the CD35 polypeptide has the amino acid sequence set forth in SEQ ID NO: 57, which is provided below:

(SEQ ID NO: 57)
RVKFSRSADAPAYQQGQNQLYNELNLGRREEYDVLDKRRGRDPE
MGGKPRRKNPQEGLYNELQKDKMAEAYSEIGMKGERRRGKGHDG
LYQGLSTATKDTYDALHMQALPPR

In accordance with the presently disclosed subject matter, a “CD3ζ nucleic acid molecule” refers to a polynucleotide encoding a CD3ζ polypeptide. In certain embodiments, the CD3 nucleic acid molecule encoding the CD3ζ polypeptide (SEQ ID NO: 56) comprised in the intracellular domain of the presently disclosed CAR comprises a nucleotide sequence as set forth in SEQ ID NO: 58 as provided below.

(SEQ ID NO: 58)
AGAGTGAAGTTCAGCAGGAGCGCAGAGCCCCCCGCGTACCAGCA
GGGCCAGAACCAGCTCTATAACGAGCTCAATCTAGGACGAAGAG
AGGAGTACGATGTTTTGGACAAGAGACGTGGCCGGGACCCTGAG
ATGGGGGGAAAGCCGAGAAGGAAGAACCCTCAGGAAGGCCTGTA
CAATGAACTGCAGAAAGATAAGATGGCGGAGGCCTACAGTGAGA
TTGGGATGAAAGGCGAGCGCCGGAGGGGCAAGGGGCACGATGGC
CTTTACCAGGGTCTCAGTACAGCCACCAAGGACACCTACGACGC
CCTTCACATGCAGGCCCTGCCCCCTCGCG

In certain embodiments, the CD35 nucleic acid molecule encoding the CD35 polypeptide (SEQ ID NO: 57) comprised in the intracellular domain of the presently disclosed CAR comprises a nucleotide sequence as set forth in SEQ ID NO: 59 as provided below.

(SEQ ID NO: 59)
AGAGTGAAGTTCAGCAGGAGCGCAGACGCCCCCGCGTACCAGCA
GGGCCAGAACCAGCTCTATAACGAGCTCAATCTAGGACGAAGAG
AGGAGTACGATGTTTTGGACAAGAGACGTGGCCGGGACCCTGAG
ATGGGGGGAAAGCCGAGAAGGAAGAACCCTCAGGAAGGCCTGTA
CAATGAACTGCAGAAAGATAAGATGGCGGAGGCCTACAGTGAGA
TTGGGATGAAAGGCGAGCGCCGGAGGGGCAAGGGGCACGATGGC
CTTTACCAGGGTCTCAGTACAGCCACCAAGGACACCTACGACGC
CCTTCACATGCAGGCCCTGCCCCCTCGCTAA

In certain non-limiting embodiments, an intracellular domain of the CAR further comprises at least one signaling region. The at least one signaling region can include a CD28 polypeptide, a 4-1BB polypeptide, an OX40 polypeptide, an ICOS polypeptide, a DAP-10 polypeptide, a PD-1 polypeptide, a CTLA-4 polypeptide, a LAG-3 polypeptide, a 2B4 polypeptide, a BTLA polypeptide, a synthetic peptide (not based on a protein associated with the immune response), or a combination thereof.

In certain embodiments, the signaling region is a co-stimulatory signaling region.

In certain embodiments, the co-stimulatory signaling region comprises at least one co-stimulatory molecule, which can provide optimal lymphocyte activation. As used herein, “co-stimulatory molecules” refer to cell surface molecules other than antigen receptors or their ligands that are required for an efficient response of lymphocytes to antigen. The at least one co-stimulatory signaling region can include a CD28 polypeptide, a 4-1BB polypeptide, an OX40 polypeptide, an ICOS polypeptide, a DAP-10 polypeptide, or a combination thereof. The co-stimulatory molecule can bind to a co-stimulatory ligand, which is a protein expressed on cell surface that upon binding to its receptor produces a co-stimulatory response, i.e., an intracellular response that effects the stimulation provided when an antigen binds to its CAR molecule. Co-stimulatory ligands, include, but are not limited to CD80, CD86, CD70, OX40L, 4-1BBL, CD48, TNFRSF14, and PD-L1. As one example, a 4-1BB ligand (i.e., 4-1BBL) may bind to 4-1BB (also known as “CD 137”) for providing an intracellular signal that in combination with a CAR signal induces an effector cell function of the CAR⁺ T cell. CARs comprising an intracellular domain that comprises a co-stimulatory signaling region comprising 4-1BB, ICOS or DAP-10 are disclosed in U.S. Pat. No. 7,446,190, which is herein incorporated by reference in its entirety. In certain embodiments, the intracellular domain of the CAR comprises a co-stimulatory signaling region that comprises a CD28 polypeptide. In certain embodiments, the intracellular domain of the CAR comprises a co-stimulatory signaling region that comprises two co-stimulatory molecules: CD28 and 4-1BB or CD28 and OX40.

4-1BB can act as a tumor necrosis factor (TNF) ligand and have stimulatory activity. The 4-1BB polypeptide can have an amino acid sequence that is at least about 85%, about 90%, about 95%, about 96%, about 97%, about 98%, about 99% or 100% homologous to the sequence having a UniProtKB Reference No: P41273 or NCBI Reference No: NP_001552 (SEQ ID NO: 60) or fragments thereof, and/or may optionally comprise up to one or up to two or up to three conservative amino acid substitutions.

SEQ ID NO: 60 is provided below:

(SEQ ID NO: 60)
MGNSCYNIVATLLLVLNFERTRSLQDPCSNCPAGTFCDNNRNQICSPCPP
NSFSSAGGQRTCDICRQCKGVFRTRKECSSTSNAECDCTPGFHCLGAGCS
MCEQDCKQGQELTKKGCKDCCFGTFNDQKRGICRPWTNCSLDGKSVLVNG
TKERDVVCGPSPADLSPGASSVTPPAPAREPGHSPQIISFFLALTSTALL
FLLFFLTLRFSVVKRGRKKLLYIFKQPFMRPVQTTQEEDGCSCRFPEEEE
GGCEL

In certain embodiments, the 4-1BB co-stimulatory domain has the amino acid sequence set forth in SEQ ID NO: 61, which is provided below:

(SEQ ID NO: 61)
KRGRKKLLYIFKQPFMRPVQTTQEEDGCSCRFPEEEEGGCEL

In accordance with the presently disclosed subject matter, a “4-1BB nucleic acid molecule” refers to a polynucleotide encoding a 4-1BB polypeptide. In certain embodiments, the 4-1BB nucleic acid molecule encoding the 4-1BB polypeptide (SEQ ID NO: 61) comprised in the intracellular domain of the presently disclosed CAR comprises a nucleotide sequence as set forth in SEQ ID NO: 62 as provided below.

(SEQ ID NO: 62)
AAACGGGGCAGAAAGAAGCTCCTGTATATATTCAAACAACCATTTATGAG
ACCAGTACAAACTACTCAAGAGGAAGATGGCTGTAGCTGCCGATTTCCAG
AAGAAGAAGAAGGAGGATGTGAACTG

An OX40 polypeptide can have an amino acid sequence that is at least about 85%, about 90%, about 95%, about 96%, about 97%, about 98%, about 99% or 100% homologous to the sequence having a UniProtKB Reference No: P43489 or NCBI Reference No: NP_003318 (SEQ ID NO: 63), or fragments thereof, and/or may optionally comprise up to one or up to two or up to three conservative amino acid substitutions.

SEQ ID NO: 63 is provided below:

(SEQ ID NO: 63)
MCVGARRLGRGPCAALLLLGLGLSTVTGLHCVGDTYPSNDRCCHECRPGN
GMVSRCSRSQNTVCRPCGPGFYNDVVSSKPCKPCTWCNLRSGSERKQLCT
ATQDTVCRCRAGTQPLDSYKPGVDCAPCPPGHFSPGDNQACKPWTNCTLA
GKHTLQPASNSSDAICEDRDPPATQPQETQGPPARPITVQPTEAWPRTSQ
GPSTRPVEVPGGRAVAAILGLGLVLGLLGPLAILLALYLLRRDQRLPPDA
HKPPGGGSFRTPIQEEQADAHSTLAKI

In accordance with the presently disclosed subject matter, an “OX40 nucleic acid molecule” refers to a polynucleotide encoding an OX40 polypeptide.

An ICOS polypeptide can have an amino acid sequence that is at least about 85%, about 90%, about 95%, about 96%, about 97%, about 98%, about 99% or 100% homologous to the sequence having a NCBI Reference No: NP_036224 (SEQ ID NO: 64) or fragments thereof, and/or may optionally comprise up to one or up to two or up to three conservative amino acid substitutions.

SEQ ID NO: 64 is provided below:

(SEQ ID NO: 64)
MKSGLWYFFLFCLRIKVLTGEINGSANYEMFIFHNGGVQILCKYPDIVQQ
FKMQLLKGGQILCDLTKTKGSGNTVSIKSLKFCHSQLSNNSVSFFLYNLD
HSHANYYFCNLSIFDPPPFKVTLTGGYLHIYESQLCCQLKFWLPIGCAAF
VVVCILGCILICWLTKKKYSSSVHDPNGEYMFMRAVNTAKKSRLTDVTL-

In accordance with the presently disclosed subject matter, an “ICOS nucleic acid molecule” refers to a polynucleotide encoding an ICOS polypeptide.

CTLA-4 is an inhibitory receptor expressed by activated T cells, which when engaged by its corresponding ligands (CD80 and CD86; B7-1 and B7-2, respectively), mediates activated T cell inhibition or anergy. In both preclinical and clinical studies, CTLA-4 blockade by systemic antibody infusion, enhanced the endogenous anti-tumor response albeit, in the clinical setting, with significant unforeseen toxicities.

CTLA-4 contains an extracellular V domain, a transmembrane domain, and a cytoplasmic tail. Alternate splice variants, encoding different isoforms, have been characterized. The membrane-bound isoform functions as a homodimer interconnected by a disulfide bond, while the soluble isoform functions as a monomer. The intracellular domain is similar to that of CD28, in that it has no intrinsic catalytic activity and contains one YVKM motif (SEQ ID NO: 65) able to bind PI3K, PP2A and SHP-2 and one proline-rich motif able to bind SH3 containing proteins. One role of CTLA-4 in inhibiting T cell responses seem to be directly via SHP-2 and PP2A dephosphorylation of TCR-proximal signaling proteins such as CD3 and LAT. CTLA-4 can also affect signaling indirectly via competing with CD28 for CD80/86 binding. CTLA-4 has also been shown to bind and/or interact with PI3K, CD80, AP2M1, and PPP2R5A.

In accordance with the presently disclosed subject matter, a CTLA-4 polypeptide can have an amino acid sequence that is at least about 85%, about 90%, about 95%, about 96%, about 97%, about 98%, about 99% or about 100% homologous to UniProtKB/Swiss-Prot Ref. No.: P16410.3 (SEQ ID NO: 66) (homology herein may be determined using standard software such as BLAST or FASTA) or fragments thereof, and/or may optionally comprise up to one or up to two or up to three conservative amino acid substitutions.

SEQ ID NO: 66 is provided below:

(SEQ ID NO: 66)
MACLGFQRHKAQLNLATRTWPCTLLFFLLFIPVFCKAMHVAQPAVVLASS
RGIASFVCEYASPGKATEVRVTVLRQADSQVTEVCAATYMMGNELTFLDD
SICTGTSSGNQVNLTIQGLRAMDTGLYICKVELMYPPPYYLGIGNGTQIY
VIDPEPCPDSDFLLWILAAVSSGLFFYSFLLTAVSLSKMLKKRSPLTTGV
YVKMPPTEPECEKQFQPYFIPIN

In accordance with the presently disclosed subject matter, a “CTLA-4 nucleic acid molecule” refers to a polynucleotide encoding a CTLA-4 polypeptide.

PD-1 is a negative immune regulator of activated T cells upon engagement with its corresponding ligands PD-L1 and PD-L2 expressed on endogenous macrophages and dendritic cells. PD-1 is a type I membrane protein of 268 amino acids. PD-1 has two ligands, PD-L1 and PD-L2, which are members of the B7 family. The protein's structure comprises an extracellular IgV domain followed by a transmembrane region and an intracellular tail. The intracellular tail contains two phosphorylation sites located in an immunoreceptor tyrosine-based inhibitory motif and an immunoreceptor tyrosine-based switch motif, that PD-1 negatively regulates TCR signals. SHP-I and SHP-2 phosphatases bind to the cytoplasmic tail of PD-1 upon ligand binding. Upregulation of PD-L1 is one mechanism tumor cells may evade the host immune system. In pre-clinical and clinical trials, PD-1 blockade by antagonistic antibodies induced anti-tumor responses mediated through the host endogenous immune system. In accordance with the presently disclosed subject matter, a PD-1 polypeptide can have an amino acid sequence that is at least about 85%, about 90%, about 95%, about 96%, about 97%, about 98%, about 99% or about 100% homologous to NCBI Reference No: NP_005009.2 (SEQ ID NO: 67) or fragments thereof, and/or may optionally comprise up to one or up to two or up to three conservative amino acid substitutions.

SEQ ID NO: 67 is provided below:

(SEQ ID NO: 67)
MQIPQAPWPVVWAVLQLGWRPGWFLDSPDRPWNPPTFSPALLVVTEGDNA
TFTCSFSNTSESFVLNWYRMSPSNQTDKLAAFPEDRSQPGQDCRFRVTQL
PNGRDFHMSVVRARRNDSGTYLCGAISLAPKAQIKESLRAELRVTERRAE
VPTAHPSPSPRPAGQFQTLVVGVVGGLLGSLVLLVWVLAVICSRAARGTI
GARRTGQPLKEDPSAVPVFSVDYGELDFQWREKTPEPPVPCVPEQTEYAT
IVFPSGMGTSSPARRGSADGPRSAQPLRPEDGHCSWPL

In accordance with the presently disclosed subject matter, a “PD-1 nucleic acid molecule” refers to a polynucleotide encoding a PD-1 polypeptide.

Lymphocyte-activation protein 3 (LAG-3) is a negative immune regulator of immune cells. LAG-3 belongs to the immunoglobulin (Ig) superfamily and contains 4 extracellular Ig-like domains. The LAG3 gene contains 8 exons. The sequence data, exon/intron organization, and chromosomal localization all indicate a close relationship of LAG3 to CD4. LAG3 has also been designated CD223 (cluster of differentiation 223). In accordance with the presently disclosed subject matter, a LAG-3 polypeptide can have an amino acid sequence that is at least about 85%, about 90%, about 95%, about 96%, about 97%, about 98%, about 99% or about 100% homologous to UniProtKB/Swiss-Prot Ref. No.: P18627.5 (SEQ ID NO: 68) or fragments thereof, and/or may optionally comprise up to one or up to two or up to three conservative amino acid substitutions.

SEQ ID NO: 68 is provided below:

(SEQ ID NO: 68)
MWEAQFLGLLFLQPLWVAPVKPLQPGAEVPVVWAQEGAPAQLPCSPTIPL
QDLSLLRRAGVTWQHQPDSGPPAAAPGHPLAPGPHPAAPSSWGPRPRRYT
VLSVGPGGLRSGRLPLQPRVQLDERGRQRGDFSLWLRPARRADAGEYRAA
VHLRDRALSCRLRLRLGQASMTASPPGSLRASDWVILNCSFSRPDRPASV
HWFRNRGQGRVPVRESPHHHLAESFLFLPQVSPMDSGPWGCILTYRDGFN
VSIMYNLTVLGLEPPTPLTVYAGAGSRVGLPCRLPAGVGTRSFLTAKWTP
PGGGPDLLVTGDNGDFTLRLEDVSQAQAGTYTCHIHLQEQQLNATVTLAI
ITVTPKSFGSPGSLGKLLCEVTPVSGQERFVWSSLDTPSQRSFSGPWLEA
QEAQLLSQPWQCQLYQGERLLGAAVYFTELSSPGAQRSGRAPGALPAGHL
LLFLILGVLSLLLLVTGAFGFHLWRRQWRPRRFSALEQGIHPPQAQSKIE
ELEQEPEPEPEPEPEPEPEPEPEQL

In accordance with the presently disclosed subject matter, a “LAG-3 nucleic acid molecule” refers to a polynucleotide encoding a LAG-3 polypeptide.

Natural Killer Cell Receptor 2B4 (2B4) mediates non-MHC restricted cell killing on NK cells and subsets of T cells. To date, the function of 2B4 is still under investigation, with the 2B4-S isoform believed to be an activating receptor, and the 2B4-L isoform believed to be a negative immune regulator of immune cells. 2B4 becomes engaged upon binding its high-affinity ligand, CD48. 2B4 contains a tyrosine-based switch motif, a molecular switch that allows the protein to associate with various phosphatases. 2B4 has also been designated CD244 (cluster of differentiation 244).

In accordance with the presently disclosed subject matter, a 2B4 polypeptide can have an amino acid sequence that is at least about 85%, about 90%, about 95%, about 96%, about 97%, about 98%, about 99% or about 100% homologous to UniProtKB/Swiss-Prot Ref. No.: Q9BZW8.2 (SEQ ID NO: 69) or fragments thereof, and/or may optionally comprise up to one or up to two or up to three conservative amino acid substitutions.

SEQ ID NO: 69 is provided below:

(SEQ ID NO: 69)
MLGQVVTLILLLLLKVYQGKGCQGSADHVVSISGVPLQLQPNSIQTKVDS
IAWKKLLPSQNGFHHILKWENGSLPSNTSNDRFSFIVKNLSLLIKAAQQQ
DSGLYCLEVTSISGKVQTATFQVFVFESLLPDKVEKPRLQGQGKILDRGR
CQVALSCLVSRDGNVSYAWYRGSKLIQTAGNLTYLDEEVDINGTHTYTCN
VSNPVSWESHTLNLTQDCQNAHQEFRFWPFLVIIVILSALFLGTLACFCV
WRRKRKEKQSETSPKEFLTIYEDVKDLKTRRNHEQEQTFPGGGSTIYSMI
QSQSSAPTSQEPAYTLYSLIQPSRKSGSRKRNHSPSFNSTIYEVIGKSQP
KAQNPARLSRKELENFDVYS

In accordance with the presently disclosed subject matter, a “2B4 nucleic acid molecule” refers to a polynucleotide encoding a 2B4 polypeptide.

B- and T-lymphocyte attenuator (BTLA) expression is induced during activation of T cells, and BTLA remains expressed on Th1 cells but not Th2 cells. Like PD1 and CTLA4, BTLA interacts with a B7 homolog, B7H4. However, unlike PD-1 and CTLA-4, BTLA displays T-Cell inhibition via interaction with tumor necrosis family receptors (TNF-R), not just the B7 family of cell surface receptors. BTLA is a ligand for tumor necrosis factor (receptor) superfamily, member 14 (TNFRSF14), also known as herpes virus entry mediator (HVEM). BTLA-HVEM complexes negatively regulate T-cell immune responses. BTLA activation has been shown to inhibit the function of human CD8⁺ cancer-specific T cells. BTLA has also been designated as CD272 (cluster of differentiation 272).

In accordance with the presently disclosed subject matter, a BTLA polypeptide can have an amino acid sequence that is at least about 85%>, about 90%, about 95%, about 96%, about 97%, about 98%, about 99% or about 100% homologous to UniProtKB/Swiss-Prot Ref. No.: Q7Z6A9.3 (SEQ ID NO: 70) or fragments thereof, and/or may optionally comprise up to one or up to two or up to three conservative amino acid substitutions.

SEQ ID NO: 70 is provided below:

(SEQ ID NO: 70)
MKTLPAMLGTGKLFWVFFLIPYLDIWNIHGKESCDVQLYIKRQSEHSILA
GDPFELECPVKYCANRPHVTWCKLNGTTCVKLEDRQTSWKEEKNISFFIL
HFEPVLPNDNGSYRCSANFQSNLIESHSTTLYVTDVKSASERPSKDEMAS
RPWLLYRLLPLGGLPLLITTCFCLFCCLRRHQGKQNELSDTAGREINLVD
AHLKSEQTEASTRQNSQVLLSETGIYDNDPDLCFRMQEGSEVYSNPCLEE
NKPGIVYASLNHSVIGPNSRLARNVKEAPTEYASICVRS

In accordance with the presently disclosed subject matter, a “BTLA nucleic acid molecule” refers to a polynucleotide encoding a BTLA polypeptide.

### Engineered Immune Cells

As described herein, immune cells can be engineered to constitutively or conditionally express a receptor including an anti-U5 snRNP200 antigen binding fragment that binds to a U5 snRNP200 antigen present on the cell surface of the cancer cells, such as AML or B cell malignancies (e.g., Diffuse large B-cell lymphoma (DLBCL), B-cell acute lymphoblastic leukemia (B-ALL), Follicular lymphoma, Chronic lymphocytic leukemia (CLL)/small lymphocytic lymphoma (SLL), Mantle cell lymphoma (MCL), Extranodal marginal zone B-cell lymphoma, Nodal marginal zone B-cell lymphoma, Splenic marginal zone B-cell lymphoma, Burkitt lymphoma, Lymphoplasmacytic lymphoma (Waldenstrom macroglobulinemia), Hairy cell leukemia, Primary central nervous system (CNS) lymphoma) or lung cancer (e.g., non-small cell lung cancer). The engineered immune cells of the present technology express a receptor comprising an anti-U5 snRNP200 antigen binding fragment (e.g., scFv) that permits delivery of the immune cell to the target cancer cells. In some embodiments, the engineered immune cells provided herein express a T-cell receptor (TCR) (e.g., caTCR, or eTCR) or other cell-surface ligand that binds to a U5 snRNP200 antigen. In some embodiments, the receptor is a chimeric antigen receptor (CAR). In exemplary embodiments provided herein, the engineered immune cells provided herein express a receptor (e.g., a CAR, caTCR, or eTCR) or other cell-surface ligand that binds to a U5 snRNP200 antigen.

Provided herein are engineered immune cells (e.g., T cells) that express a U5 snRNP200-specific antigen receptor (e.g., CAR, caTCR, or eTCR) that effectively target cancer cells. The engineered immune cells (e.g., T cells) provided herein that express a U5 snRNP200-specific antigen receptor (e.g., CAR, caTCR, or eTCR) are useful in methods for eliminating cancer cells, and/or treating cancer, such as AML or B cell malignancies (e.g., Diffuse large B-cell lymphoma (DLBCL), B-cell acute lymphoblastic leukemia (B-ALL), Follicular lymphoma, Chronic lymphocytic leukemia (CLL)/small lymphocytic lymphoma (SLL), Mantle cell lymphoma (MCL), Extranodal marginal zone B-cell lymphoma, Nodal marginal zone B-cell lymphoma, Splenic marginal zone B-cell lymphoma, Burkitt lymphoma, Lymphoplasmacytic lymphoma (Waldenstrom macroglobulinemia), Hairy cell leukemia, Primary central nervous system (CNS) lymphoma) or lung cancer (e.g., non-small cell lung cancer), in a subject in need thereof.

In certain embodiments, the engineered immune cells will proliferate extensively (e.g., 100 times or more) when it encounters a U5 snRNP200 antigen at a tissue site, thus significantly increasing production of the receptor comprising the anti-U5 snRNP200 antigen binding fragment. The engineered immune cells (e.g., T cells) can be generated by in vitro transduction of immune cells with a nucleic acid encoding the receptor comprising the anti-U5 snRNP200 antigen binding fragment (e.g., CAR, caTCR, or eTCR). Further, the activity of the engineered immune cells (e.g., T cells) can be adjusted by selection of co-stimulatory molecules included in the receptor (e.g., CAR, caTCR, or eTCR).

In some embodiments, the receptor (e.g., a CAR, caTCR, or eTCR) comprises a U5 snRNP200 antigen binding fragment (e.g., scFv) comprising a VHCDR1 sequence, a VHCDR2 sequence, and a VHCDR3 sequence of GYYWS (SEQ ID NO: 17), EINHSGSTNYNPSLKS (SEQ ID NO: 18), and GRSTSPLDYYYYYMDV (SEQ ID NO: 19); or GYYWS (SEQ ID NO: 23), EINHSGSTNYNPSLKS (SEQ ID NO: 24), and GPRGMYSSSSGDY (SEQ ID NO: 25); or TYGMH (SEQ ID NO: 29), VIWYDGSNTYYADSVKG (SEQ ID NO: 30), and ARGRGYSAQGNRNRAYYFDY (SEQ ID NO: 31) respectively. Additionally or alternatively, in some embodiments, the U5 snRNP200 antigen binding fragment (e.g., scFv) comprises a V_{L}CDR1 sequence, a V_{L}CDR2 sequence, and a V_{L}CDR3 sequence of QGDFLRSYYAS (SEQ ID NO: 20), GKNKRPS (SEQ ID NO: 21), and NSRDRSGNHLV (SEQ ID NO: 22); or RASQGIRNDLG (SEQ ID NO: 26), AAVSLQS (SEQ ID NO: 27), and LQHNSYPRT (SEQ ID NO: 28); or RASQSVSSNLA (SEQ ID NO: 32), GAFTRVT (SEQ ID NO: 33), and QQYNDRPPYT (SEQ ID NO: 34) respectively.

Additionally or alternatively, in some embodiments, the amino acid sequence of the V_{H} of the anti-U5 snRNP200 antigen binding fragment (e.g., scFv) is:

(SEQ ID NO: 35)
QVQLQQWGAGLLKPSETLSLTCAVYGGSFSGYYWSWIRQPPGKGLEWIGE
INHSGSTNYNPSLKSRVTISVDTSKNQFSLKLSSVTAADTAVYYCARGRS
TSPLDYYYYYMDVWAKGTTVTVS,
or
(SEQ ID NO: 36)
QVQLQQWGAGLLKPSETLSLTCAVYGGSFSGYYWSWIRQPPGKGLEWIGE
INHSGSTNYNPSLKSRVTISVDTSKKQFSLKLSSVTAADTAVYYCARGPR
GMYSSSSGDYWGQGTLVTVS
(SEQ ID NO: 37)
QVQLVESGGGVVQPGRSLRLSCAASGFTFSTYGMHWVRQAPGKGLEWVAV
IWYDGSNTYYADSVKGRFTISRDNSKNTLYLQIKSLRAEDTAVYYCARGR
GYSAQGNRNRAYYFDYWGQGTLVTVS.

Additionally or alternatively, in some embodiments, the amino acid sequence of the V_{L} of the anti-U5 snRNP200 antigen binding fragment (e.g., scFv) is:

(SEQ ID NO: 38)
SSELTQDPAVSVALGQTVRITCQGDFLRSYYASWYQQKPGQAPVLVIFGK
NKRPSGIPDRFSGSSSGNTASLTITGAQAEDEADYYCNSRDRSGNHLVFG
GGTKLTVL,
(SEQ ID NO: 39)
DIQMTQSPSSLSASVGDRVTITCRASQGIRNDLGWYQQKPGKAPKRLIYA
AVSLQSGVPSRFSGSGSGTEFTLTISSLQPEDFATYYCLQHNSYPRTFGQ
GTKLEIK,
or
(SEQ ID NO: 40)
EIVMTQSPATLSVSPGERVILSCRASQSVSSNLAWYQQKPGQPPRLLIYG
AFTRVTGVPARFSGSGSGTEFTLTISSLQSEDFAVYYCQQYNDRPPYTFG
QGTKLEIKRAVDQ

In some embodiments, the V_{H} comprises an amino acid sequence that is at least 90%, at least 95%, or 100% identical to any one of SEQ ID NOs: 35-37 and/or the V_{L} comprises an amino acid sequence that is at least 90%, at least 95%, or 100% identical to any one of SEQ ID NOs: 38-40. In some embodiments, the antigen binding fragment specifically binds to U5 snRNP200. In certain embodiments, the antigen binding fragment is selected from the group consisting of Fab, F(ab′)2, Fab′, scFv, and Fv. The antigen binding fragment may be monoclonal, chimeric, humanized, or bispecific.

Additionally or alternatively, in some embodiments, the anti-U5 snRNP200 antigen binding fragment (e.g., scFv) comprises an amino acid sequence selected from the group consisting of:

(SEQ ID NO: 2)
QVQLQQWGAGLLKPSETLSLTCAVYGGSFSGYYWSWIRQPPGKGLEWIGE
INHSGSTNYNPSLKSRVTISVDTSKNQFSLKLSSVTAADTAVYYCARGRS
TSPLDYYYYYMDVWAKGTTVTVSGGGGSGGGGSGGGGSSSELTQDPAVSV
ALGQTVRITCQGDFLRSYYASWYQQKPGQAPVLVIFGKNKRPSGIPDRFS
GSSSGNTASLTITGAQAEDEADYYCNSRDRSGNHLVFGGGTKLTVL;
(SEQ ID NO: 6)
SSELTQDPAVSVALGQTVRITCQGDFLRSYYASWYQQKPGQAPVLVIFGK
NKRPSGIPDRFSGSSSGNTASLTITGAQAEDEADYYCNSRDRSGNHLVFG
GGTKLTVLGGGGSGGGGSGGGGSQVQLQQWGAGLLKPSETLSLTCAVYGG
SFSGYYWSWIRQPPGKGLEWIGEINHSGSTNYNPSLKSRVTISVDTSKNQ
FSLKLSSVTAADTAVYYCARGRSTSPLDYYYYYMDVWAKGTTVTVS;
(SEQ ID NO: 8)
QVQLQQWGAGLLKPSETLSLTCAVYGGSFSGYYWSWIRQPPGKGLEWIGK
INHSGSTNYNPSLKSRVTISVDTSKKQFSLKLSSVTAADTAVYYCARGPR
GMYSSSSGDYWGQGTLVTVSGGGGSGGGGSGGGGSDIQMTQSPSSLSASV
GDRVTITCRASQGIRNDLGWYQQKPGKAPKRLIYAAVSLQSGVPSRFSGS
GSGTEFTLTISSLQPEDFATYYCLQHNSYPRTFGQGTKLEIK;
(SEQ ID NO: 10)
DIQMTQSPSSLSASVGDRVTITCRASQGIRNDLGWYQQKPGKAPKRLIYA
AVSLQSGVPSRFSGSGSGTEFTLTISSLQPEDFATYYCLQHNSYPRTFGQ
GTKLEIKGGGGSGGGGSGGGGSQVQLQQWGAGLLKPSETLSLTCAVYGGS
FSGYYWSWIRQPPGKGLEWIGEINHSGSTNYNPSLKSRVTISVDTSKKQF
SLKLSSVTAADTAVYYCARGPRGMYSSSSGDYWGQGTLVTVS;
(SEQ ID NO: 12)
QVQLVESGGGVVQPGRSLRLSCAASGFTFSTYGMHWVRQAPGKGLEWVAV
IWYDGSNTYYADSVKGRFTISRDNSKNTLYLQIKSLRAEDTAVYYCARGR
GYSAQGNRNRAYYFDYWGQGTLVTVSGGGGSGGGGSGGGGSEIVMTQSPA
TLSVSPGERVILSCRASQSVSSNLAWYQQKPGQPPRLLIYGAFTRVTGVP
ARFSGSGSGTEFTLTISSLQSEDFAVYYCQQYNDRPPYTFGQGTKLEIKR
AVDQ;
and
(SEQ ID NO: 14)
EIVMTQSPATLSVSPGERVILSCRASQSVSSNLAWYQQKPGQPPRLLIYG
AFTRVTGVPARFSGSGSGTEFTLTISSLQSEDFAVYYCQQYNDRPPYTFG
QGTKLEIKRAVDQGGGGGGGGSGGGGSQVQLVESGGGVVQPGRSLRLSCA
ASGFTFSTYGMHWVRQAPGKGLEWVAVIWYDGSNTYYADSVKGRFTISRD
NSKNTLYLQIKSLRAEDTAVYYCARGRGYSAQGNRNRAYYFDYWGQGTLV
TVS.

Additionally or alternatively, in some embodiments, the anti-U5 snRNP200 antigen binding fragment (e.g., scFv) comprises an amino acid sequence that has at least 80%, 85%, 90%, 95%, 96%, 97%, 98%, or 99% sequence identity to any one of SEQ ID NOs: 2, 6, 8, 10, 12, or 14. In some embodiments, the anti-U5 snRNP200 antigen binding fragment (e.g., scFv) comprises an amino acid sequence that is about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NOs: 2, 6, 8, 10, 12, or 14. In some embodiments, the anti-U5 snRNP200 antigen binding fragment is an scFv, a Fab, or a (Fab) 2.

Additionally or alternatively, in some embodiments, the anti-U5 snRNP200 antigen binding fragment (e.g., scFv) is encoded by a nucleic acid sequence selected from the group consisting of:

(SEQ ID NO: 1)
gaggtgcagctacagcagtggggcgcaggactgttgaagccttcggagac
cctgtccctcacctgcgctgtctatggtgggtccttcagtggttactact
ggagctggatccgccagcccccagggaaggggctggagtggattggggaa
atcaatcatagtggaagcaccaactacaacccgtccctcaagagtcgagt
caccatatcagtagacacgtccaagaaccagttctccctgaagctgagct
ctgtgaccgccgcggacacggctgtgtattactgtgcgaggggccgtagt
accagcccgctcgactactactactactacatggacgtctgggccaaagg
gaccacggtcaccgtctccGGTGGAGGTGGATCAGGTGGAGGTGGATCTG
GTGGAGGTGGATCTtcttctgagctgactcaggaccctgctgtgtctgtg
gccttgggacagacagtcaggatcacatgccaaggagacttcctcagaag
ctattatgcaagctggtaccagcagaagccaggacaggcccctgtacttg
tcatctttggtaaaaacaagcggccctcagggatcccagaccgattctct
ggctccagctcaggaaacacagcttccttgaccatcactggggctcaggc
ggaagatgaggctgactattactgtaactcccgggaccgcagtggtaacc
acctggtgttcggcggagggaccaagctgaccgtccta;
(SEQ ID NO: 5)
ccttctgagctgactcaggaccctgctgtgtctgtggccttgggacagac
agtcaggatcacatgccaaggagacttcctcagaagctattatgcaagct
ggtaccagcagaagccaggacaggcccctgtacttgtcatctttggtaaa
aacaagcggccctcagggatcccagaccgattctctggctccagctcagg
aaacacagcttccttgaccatcactggggctcaggcggaagatgaggctg
actattactgtaactcccgggaccgcagtggtaaccacctggtgttcggc
ggagggaccaagctgaccgtcctaGGTGGAGGTGGATCAGGTGGAGGTGG
ATCTGGTGGAGGTGGATCTcaggtgcagctacagcagtggggcgcaggac
tgttgaagccttcggagaccctgtccctcacctgcgctgtctatggtggg
tccttcagtggttactactggagctggatccgccagcccccagggaaggg
gctggagtggattggggaaatcaatcatagtggaagcaccaactacaacc
cgtccctcaagagtcgagtcaccatatcagtagacacgtccaagaaccag
ttctccctgaagctgagctctgtgaccgccgcggacacggctgtgtatta
ctgtgcgaggggccgtagtaccagcccgctcgactactactactactaca
tggacgtctgggccaaagggaccacggtcaccgtctcc;
(SEQ ID NO: 7)
caggtgcagctacagcagtggggcgcaggactgttgaagccttcggagac
cctgtccctcacctgcgctgtctatggtgggtccttcagtggttactact
ggagctggatccgccagcccccagggaaggggctggagtggattggggaa
atcaatcatagtggaagcaccaactacaacccgtccctcaagagtcgagt
caccatatcagtagacacgtccaagaagcagttctccctgaagctgagct
ctgtgaccgccgcggacacggctgtgtattattgtgcgagaggcccccgg
ggcatgtatagcagctcgtccggggactactggggccagggaaccctggt
caccgtctccGGTGGAGGTGGATCAGGTGGAGGTGGATCTGGTGGAGGTG
GATCTgacatccagatgacccagtctccatcctccctgtctgcatctgta
ggagacagagtcaccatcacttgccgggcaagtcagggcattagaaatga
tttaggctggtatcagcagaaaccagggaaagcccctaagcgcctgatct
atgctgcagtcagtttgcaaagtggggtcccatcaaggttcagcggcagt
ggatctgggacagaattcactctcacaatcagcagcctgcagcctgaaga
ttttgcaacttattactgtctacagcataatagttaccctcggacttttg
gccaggggaccaagctggagatcaaa;
(SEQ ID NO: 9)
tatatacaaatgacgcagagtccttcaagtctttcagcctccgtagggga
tcgggtaacgatcacatgtcgagcatcccaaggcattcggaatgacttgg
gatggtatcaacagaagccgggcaaggccccaaagaggttgatatatgct
gctgtttcactgcaatccggcgtaccttccagattttcaggaagtggaag
gggaccgaatttactctgacgataagtagcctccaacctgaagacttcgc
cacctactattgtttgcaacataattcttatccaaggacttttgggcaag
gcacgaaacttgaaattaagGGTGGAGGTGGATCAGGTGGAGGTGGATCT
GGTGGAGGTGGATCTcaggtgcagctacagcagtggggcgcaggactgtt
gaagccttcggagaccctgtccctcacctgcgctgtctatggtgggtcct
tcagtggttactactggagctggatccgccagcccccagggaaggggctg
gagtggattggggaaatcaatcatagtggaagcaccaactacaacccgtc
cctcaagagtcgagtcaccatatcagtagacacgtccaagaagcagttct
ccctgaagctgagctctgtgaccgccgcggacacggctgtgtattattgt
gcgagaggcccccggggcatgtatagcagctcgtccggggactactgggg
ccagggaaccctggtcaccgtctcc;
(SEQ ID NO: 11)
gaggtgcagctggtggagtctgggggaggcgtggtccagcctgggaggtc
cctgagactctcctgtgcagcgtctggattcaccttcagtacctatggca
tgcactgggtccgccaggctccaggcaaggggcttgagtgggtggcagtt
atatggtatgatggaagtaatacatactatgcagactccgtgaagggccg
attcaccatctccagagacaattccaagaacacactgtatctgcaaataa
agagcctgagagccgaggacacggctgtctattactgtgcgagaggccgt
ggatatagtgcccaagggaatcggaatagggcttactactttgactactg
gggccagggaaccctggtcaccgtctccGGTGGAGGTGGATCAGGTGGAG
GTGGATCTGGTGGAGGTGGATCTgaaatagtgatgacgcagtctccagcc
accctgtctgtgtctccaggggaaagggtcatcctctcctgcagggccag
tcagagtgttagcagcaacttagcctggtaccagcagaaacctggccagc
ctcccaggctcctcatctatggtgcattcacgagggtcactggtgtccca
gccaggttcagtggcagtgggtctgggacagaattcactctcaccatcag
cagcctgcagtctgaagattttgcagtttattactgtcagcagtacaatg
accggcccccgtacacttttggccaggggaccaagctggagatcaaacgg
gctgtcgaccaa;
and
(SEQ ID NO: 13)
gaaatagtgatgacgcagtctccagccaccctgtctgtgtctccagggga
aagggtcatcctctcctgcagggccagtcagagtgttagcagcaacttag
cctggtaccagcagaaacctggccagcctcccaggctcctcatctatggt
gcattcacgagggtcactggtgtcccagccaggttcagtggcagtgggtc
tgggacagaattcactctcaccatcagcagcctgcagtctgaagattttg
cagtttattactgtcagcagtacaatgaccggcccccgtacacttttggc
caggggaccaagctggagatcaaaCGGGCTGTCGACCAAGGAGGTGGAGG
ATCTGGGGGTGGAGGTTCTGGTGGGGGTGGATCTcaggtgcagctggtgg
agtctgggggaggcgtggtccagccagggcggtccctgagactctcctgt
gcagcgtctggattcaccttcagtacctatggcatgcactgggtccgcca
ggctccaggcaaggggcttgagtgggggcagttatatggtatgatggaag
taatacatactatgcagactccgtgaagggccgattcaccatctccagag
acaattccaagaacacactgtatctgcaaataaagagcctgagagccgag
gacacggctgtctattactgtgcgagaggccgtggatatagtgcccaagg
gaatcggaatagggcttactactttgactactggggccagggaaccctgg
tcaccgtctcc.

Linker sequences are capitalized

Additionally or alternatively, in some embodiments, the anti-U5 snRNP200 antigen binding fragment (e.g., scFv) is encoded by a nucleic acid sequence that has at least 80%, 85%, 90%, 95%, 96%, 97%, 98%, or 99% sequence identity to any one of SEQ ID NOs: 1, 5, 7, 9, 11 or 13. In some embodiments, the anti-U5 snRNP200 antigen binding fragment (e.g., scFv) is encoded by a nucleic acid that is about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NOs: 1, 5, 7, 9, 11 or 13.

Additionally or alternatively, in certain embodiments, the U5 snRNP200-specific receptor (e.g., a CAR, caTCR, or eTCR) of the present technology and a reporter or selection marker (e.g., GFP, LNGFR) or an IL-18 polypeptide are expressed as a single polypeptide linked by a self-cleaving linker, such as a P2A linker. In certain embodiments, the receptor (e.g., a CAR, caTCR, or eTCR) and a reporter or selection marker (e.g., GFP, LNGFR) or an IL-18 polypeptide are expressed as two separate polypeptides.

In any and all of the preceding embodiments, the CAR comprises an extracellular binding fragment (e.g., anti-U5 snRNP200 scFv) that specifically binds to a U5 snRNP200 antigen or polypeptide, a transmembrane domain comprising a CD28 polypeptide and/or a CD8 polypeptide, and an intracellular domain comprising a CD3ζ polypeptide and optionally a co-stimulatory signaling region disclosed herein. The CAR may also comprise a signal peptide or a leader sequence operably linked to the N-terminus of the extracellular U5 snRNP200 binding fragment. The signal peptide comprises amino acids having the sequence set forth in SEQ ID NO: 41, SEQ ID NO: 43, SEQ ID NO: 45 or SEQ ID NO: 47.

Additionally or alternatively, in some embodiments, the nucleic acid encoding the receptor (e.g., a CAR, caTCR, or eTCR) of the present technology is operably linked to an inducible promoter. In some embodiments, the nucleic acid encoding the receptor (e.g., a CAR, caTCR, or eTCR) of the present technology is operably linked to a constitutive promoter.

In some embodiments, the inducible promoter is a synthetic Notch promoter that is activatable in an immune cell including the receptor (e.g., CAR, caTCR, or eTCR), where the intracellular domain of the receptor contains a transcriptional regulator that is released from the membrane when engagement of the receptor (e.g., CAR, caTCR, or eTCR) with the U5 snRNP200 antigen/polypeptide induces intramembrane proteolysis (see, e.g., Morsut et al., Cell 164 (4): 780-791 (2016). Accordingly, further transcription of the U5 snRNP200-specific receptor (e.g., a CAR, caTCR, or eTCR) is induced upon binding of the engineered immune cell with the U5 snRNP200 antigen/polypeptide.

The presently disclosed subject matter also provides isolated nucleic acid molecules encoding the receptor (e.g., a CAR, caTCR, or eTCR) constructs described herein or a functional portion thereof.

In certain embodiments, the isolated nucleic acid molecule encodes an anti-U5 snRNP200-targeted CAR comprising (a) an U5 snRNP200 binding fragment (e.g., an anti-U5 snRNP200 scFv) that specifically binds to a U5 snRNP200 antigen, (b) a transmembrane domain comprising a CD8 polypeptide or CD28 polypeptide, and (c) an intracellular domain comprising a CD28 polypeptide and/or a CD3ζ polypeptide, and optionally one or more of (i) a co-stimulatory signaling region disclosed herein, (ii) a P2A self-cleaving peptide, and (iii) a reporter or selection marker (e.g., GFP, LNGFR) or an IL-18 polypeptide provided herein. The at least one co-stimulatory signaling region can include a CD28 polypeptide, a 4-1BB polypeptide, an OX40 polypeptide, an ICOS polypeptide, a DAP-10 polypeptide, a PD-1 polypeptide, a CTLA-4 polypeptide, a LAG-3 polypeptide, a 2B4 polypeptide, a BTLA polypeptide, a synthetic peptide (not based on a protein associated with the immune response), or a combination thereof. In certain embodiments, the isolated nucleic acid molecule encodes an U5 snRNP200-targeted CAR comprising a U5 snRNP200 binding fragment (e.g., an anti-U5 snRNP200 scFv) that specifically binds to a U5 snRNP200 antigen/polypeptide, fused to a synthetic Notch transmembrane domain and an intracellular cleavable transcription factor. In certain embodiments, the present disclosure provides an isolated nucleic acid molecule encoding a U5 snRNP200-specific CAR that is inducible by release of the transcription factor of a synthetic Notch system.

In certain embodiments, the isolated nucleic acid molecule encodes a functional portion of a presently disclosed receptor (e.g., a CAR, caTCR, or eTCR) constructs. As used herein, the term “functional portion” refers to any portion, part or fragment of a receptor (e.g., a CAR, caTCR, or eTCR), which portion, part or fragment retains the biological activity of the parent receptor (e.g., a CAR, caTCR, or eTCR). For example, functional portions encompass the portions, parts or fragments of a U5 snRNP200-specific receptor (e.g., a CAR, caTCR, or eTCR) that retains the ability to recognize a target cancer cell, to treat cancer to a similar, same, or even a higher extent as the parent receptor (e.g., a CAR, caTCR, or eTCR). In certain embodiments, an isolated nucleic acid molecule encoding a functional portion of a U5 snRNP200-specific receptor (e.g., a CAR, caTCR, or eTCR) can encode a protein comprising, e.g., about 10%, about 20%, about 25%, about 30%, about 35%, about 40%, about 45%, about 50%, about 55%, about 60%, about 65%, about 70%, about 75%, about 80%, about 85%, about 90%, and about 95%, or more of the parent receptor (e.g., a CAR, caTCR, or eTCR).

The presently disclosed subject matter provides engineered immune cells expressing a U5 snRNP200-specific receptor (e.g., a CAR, caTCR, or eTCR) or other ligand that comprises an extracellular antigen-binding domain, a transmembrane domain and an intracellular domain, where the extracellular antigen-binding domain specifically binds a U5 snRNP200 antigen/polypeptide. In certain embodiments immune cells can be transduced with a presently disclosed receptor (e.g., a CAR, caTCR, or eTCR) constructs such that the cells express the receptor (e.g., a CAR, caTCR, or eTCR). The presently disclosed subject matter also provides methods of using such cells for the treatment of cancer such as AML or B cell malignancies (e.g., Diffuse large B-cell lymphoma (DLBCL), B-cell acute lymphoblastic leukemia (B-ALL), Follicular lymphoma, Chronic lymphocytic leukemia (CLL)/small lymphocytic lymphoma (SLL), Mantle cell lymphoma (MCL), Extranodal marginal zone B-cell lymphoma, Nodal marginal zone B-cell lymphoma, Splenic marginal zone B-cell lymphoma, Burkitt lymphoma, Lymphoplasmacytic lymphoma (Waldenstrom macroglobulinemia), Hairy cell leukemia, Primary central nervous system (CNS) lymphoma) or lung cancer (e.g., non-small cell lung cancer).

The presently disclosed subject matter also provides methods of using such cells for the treatment of a tumor. The engineered immune cells of the presently disclosed subject matter can be cells of the lymphoid lineage or myeloid lineage. Examples of myeloid cells include but are not limited to, mast cells, monocytes, macrophages, dendritic cells, eosinophils, neutrophils, basophils. The lymphoid lineage, comprising B, T, and natural killer (NK) cells, provides for the production of antibodies, regulation of the cellular immune system, detection of foreign agents in the blood, detection of cells foreign to the host, and the like. Non-limiting examples of immune cells of the lymphoid lineage include T cells, Natural Killer (NK) cells, embryonic stem cells, and pluripotent stem cells (e.g., those from which lymphoid cells can be differentiated). T cells can be lymphocytes that mature in the thymus and are chiefly responsible for cell-mediated immunity. T cells are involved in the adaptive immune system. The T cells of the presently disclosed subject matter can be any type of T cells, including, but not limited to, T helper cells, cytotoxic T cells, memory T cells (including central memory T cells, stem-cell-like memory T cells (or stem-like memory T cells)), and two types of effector memory T cells: e.g., TEM cells and TEMRA cells, Regulatory T cells (also known as suppressor T cells), Natural killer T cells, Mucosal associated invariant T cells, and γδ T cells. Cytotoxic T cells (CTL or killer T cells) are a subset of T lymphocytes capable of inducing the death of infected somatic or tumor cells.

Natural killer (NK) cells can be lymphocytes that are part of cell-mediated immunity and act during the innate immune response. NK cells do not require prior activation in order to perform their cytotoxic effect on target cells.

The engineered immune cells of the presently disclosed subject matter can express an extracellular U5 snRNP200 binding domain (e.g., an anti-U5 snRNP200 scFv, an anti-U5 snRNP200 Fab that is optionally crosslinked, an anti-U5 snRNP200 F(ab)₂) that specifically binds to a U5 snRNP200 antigen, for the treatment of cancer such as AML or B cell malignancies (e.g., Diffuse large B-cell lymphoma (DLBCL), B-cell acute lymphoblastic leukemia (B-ALL), Follicular lymphoma, Chronic lymphocytic leukemia (CLL)/small lymphocytic lymphoma (SLL), Mantle cell lymphoma (MCL), Extranodal marginal zone B-cell lymphoma, Nodal marginal zone B-cell lymphoma, Splenic marginal zone B-cell lymphoma, Burkitt lymphoma, Lymphoplasmacytic lymphoma (Waldenstrom macroglobulinemia), Hairy cell leukemia, Primary central nervous system (CNS) lymphoma) or lung cancer (e.g., non-small cell lung cancer). Such engineered immune cells can be administered to a subject (e.g., a human subject) in need thereof for the treatment of cancer such as AML or B cell malignancies (e.g., Diffuse large B-cell lymphoma (DLBCL), B-cell acute lymphoblastic leukemia (B-ALL), Follicular lymphoma, Chronic lymphocytic leukemia (CLL)/small lymphocytic lymphoma (SLL), Mantle cell lymphoma (MCL), Extranodal marginal zone B-cell lymphoma, Nodal marginal zone B-cell lymphoma, Splenic marginal zone B-cell lymphoma, Burkitt lymphoma, Lymphoplasmacytic lymphoma (Waldenstrom macroglobulinemia), Hairy cell leukemia, Primary central nervous system (CNS) lymphoma) or lung cancer (e.g., non-small cell lung cancer). In some embodiments, the immune cell is a lymphocyte, such as a T cell, a B cell or a natural killer (NK) cell. In certain embodiments, the engineered immune cell is a T cell. The T cell can be a CD4⁺ T cell or a CD8⁺ T cell. In certain embodiments, the T cell is a CD4⁺ T cell. In certain embodiments, the T cell is a CD8⁺ T cell.

The engineered immune cells of the present disclosure can further include at least one recombinant or exogenous co-stimulatory ligand. For example, the engineered immune cells of the present disclosure can be further transduced with at least one co-stimulatory ligand, such that the engineered immune cells co-expresses or is induced to co-express the U5 snRNP200-specific receptor (e.g., a CAR, caTCR, or eTCR) and the at least one co-stimulatory ligand. The interaction between the U5 snRNP200-specific receptor (e.g., a CAR, caTCR, or eTCR) and the at least one co-stimulatory ligand provides a non-antigen-specific signal important for full activation of an immune cell (e.g., T cell). Co-stimulatory ligands include, but are not limited to, members of the tumor necrosis factor (TNF) superfamily, and immunoglobulin (Ig) superfamily ligands. TNF is a cytokine involved in systemic inflammation and stimulates the acute phase reaction. Its primary role is in the regulation of immune cells. Members of TNF superfamily share a number of common features. The majority of TNF superfamily members are synthesized as type II transmembrane proteins (extracellular C-terminus) containing a short cytoplasmic segment and a relatively long extracellular region. TNF superfamily members include, without limitation, nerve growth factor (NGF), CD40L (CD40L)/CD 154, CD137L/4-1BBL, TNF-α, CD134L/OX40L/CD252, CD27L/CD70, Fas ligand (FasL), CD30L/CD153, tumor necrosis factor beta (TNFP)/lymphotoxin-alpha (LT-α), lymphotoxin-beta (LT-β), CD257/B cell-activating factor (BAFF)/Blys/THANK/Tall-1, glucocorticoid-induced TNF Receptor ligand (GITRL), TNF-related apoptosis-inducing ligand (TRAIL), and LIGHT (TNFSF14). The immunoglobulin (Ig) superfamily is a large group of cell surface and soluble proteins that are involved in the recognition, binding, or adhesion processes of cells. These proteins share structural features with immunoglobulins-they possess an immunoglobulin domain (fold). Immunoglobulin superfamily ligands include, but are not limited to, CD80 and CD86, both ligands for CD28, or PD-L1/(B7-H1) that are ligands for PD-1. In certain embodiments, the at least one co-stimulatory ligand is selected from the group consisting of 4-1BBL, CD80, CD86, CD70, OX40L, CD48, TNFRSF14, PD-L1, and combinations thereof. In certain embodiments, the engineered immune cell comprises one recombinant co-stimulatory ligand (e.g., 4-1BBL). In certain embodiments, the engineered immune cell comprises two recombinant co-stimulatory ligands (e.g., 4-1BBL and CD80). CARs comprising at least one co-stimulatory ligand are described in U.S. Pat. No. 8,389,282, which is incorporated by reference in its entirety.

Furthermore, the engineered immune cells of the present disclosure can further comprise at least one exogenous cytokine. For example, a presently disclosed engineered immune cell can be further transduced with at least one cytokine, such that the engineered immune cell secretes the at least one cytokine as well as expresses the U5 snRNP200-specific receptor (e.g., a CAR, caTCR, or eTCR). In certain embodiments, the at least one cytokine is selected from the group consisting of IL-2, IL-3, IL-6, IL-7, IL-11, IL-12, IL-15, IL-17, and IL-21.

The engineered immune cells can be generated from peripheral donor lymphocytes, e.g., those disclosed in Sadelain, M., et al., Nat Rev Cancer 3:35-45 (2003) (disclosing peripheral donor lymphocytes genetically modified to express CARs), in Morgan, R. A. et al., Science 314:126-129 (2006) (disclosing peripheral donor lymphocytes genetically modified to express a full-length tumor antigen-recognizing T cell receptor complex comprising the α and β heterodimer), in Panelli et al., J Immunol 164:495-504 (2000); Panelli et al., J Immunol 164:4382-4392 (2000) (disclosing lymphocyte cultures derived from tumor infiltrating lymphocytes (TILs) in tumor biopsies), and in Dupont et al., Cancer Res 65:5417-5427 (2005); Papanicolaou et al., Blood 102:2498-2505 (2003) (disclosing selectively inv/Yro-expanded antigen-specific peripheral blood leukocytes employing artificial antigen-presenting cells (AAPCs) or pulsed dendritic cells). The engineered immune cells (e.g., T cells) can be autologous, non-autologous (e.g., allogeneic), or derived in vitro from engineered progenitor or stem cells.

In certain embodiments, the engineered immune cells of the present disclosure (e.g., T cells) express from about 1 to about 5, from about 1 to about 4, from about 2 to about 5, from about 2 to about 4, from about 3 to about 5, from about 3 to about 4, from about 4 to about 5, from about 1 to about 2, from about 2 to about 3, from about 3 to about 4, or from about 4 to about 5 vector copy numbers per cell of a presently disclosed U5 snRNP200-specific receptor (e.g., a CAR, caTCR, or eTCR).

For example, the higher the receptor (e.g., a CAR, caTCR, or eTCR) expression level in an engineered immune cell, the greater cytotoxicity and cytokine production the engineered immune cell exhibits. An engineered immune cell (e.g., T cell) having a high U5 snRNP200-specific receptor (e.g., a CAR, caTCR, or eTCR) expression level can induce antigen-specific cytokine production or secretion and/or exhibit cytotoxicity to a tissue or a cell having a low expression level of U5 snRNP200, e.g., about 2,000 or less, about 1,000 or less, about 900 or less, about 800 or less, about 700 or less, about 600 or less, about 500 or less, about 400 or less, about 300 or less, about 200 or less, about 100 or less of U5 snRNP200 antigen binding sites/cell. Additionally or alternatively, the cytotoxicity and cytokine production of a presently disclosed engineered immune cell (e.g., T cell) are proportional to the expression level of U5 snRNP200 antigen in a target tissue or a target cell. For example, the higher the expression level of U5 snRNP200 antigen in the target, the greater cytotoxicity and cytokine production the engineered immune cell exhibits.

The unpurified source of immune cells may be any source known in the art, such as the bone marrow, fetal, neonate or adult or other hematopoietic cell source, e.g., fetal liver, peripheral blood or umbilical cord blood. Various techniques can be employed to separate the cells. For instance, negative selection methods can remove non-immune cells initially. Monoclonal antibodies are particularly useful for identifying markers associated with particular cell lineages and/or stages of differentiation for both positive and negative selections.

A large proportion of terminally differentiated cells can be initially removed by a relatively crude separation. For example, magnetic bead separations can be used initially to remove large numbers of irrelevant cells. Suitably, at least about 80%, usually at least 70% of the total hematopoietic cells will be removed prior to cell isolation.

Procedures for separation include, but are not limited to, density gradient centrifugation; resetting; coupling to particles that modify cell density; magnetic separation with antibody-coated magnetic beads; affinity chromatography; cytotoxic agents joined to or used in conjunction with a mAb, including, but not limited to, complement and cytotoxins; and panning with antibody attached to a solid matrix, e.g., plate, chip, elutriation or any other convenient technique.

Techniques for separation and analysis include, but are not limited to, flow cytometry, which can have varying degrees of sophistication, e.g., a plurality of color channels, low angle and obtuse light scattering detecting channels, impedance channels.

The cells can be selected against dead cells, by employing dyes associated with dead cells such as propidium iodide (PI). Usually, the cells are collected in a medium comprising 2% fetal calf serum (FCS) or 0.2% bovine serum albumin (BSA) or any other suitable (e.g., sterile), isotonic medium.

In some embodiments, the engineered immune cells comprise one or more additional modifications. For example, in some embodiments, the engineered immune cells comprise and express (are transduced to express) an antigen recognizing receptor that binds to a second antigen that is different than the first U5 snRNP200 antigen. The inclusion of an antigen recognizing receptor in addition to a presently disclosed receptor (e.g., a CAR, caTCR, or eTCR) on the engineered immune cell can increase the avidity of the receptor (e.g., a CAR, caTCR, or eTCR) (or the engineered immune cell comprising the same) on a target cell, especially, the receptor (e.g., a CAR, caTCR, or eTCR) is one that has a low binding affinity to a particular U5 snRNP200 antigen, e.g., a K_{d} of about 2×10⁻⁸ M or more, about 5×10⁻⁸ M or more, about 8×10⁻⁸ M or more, about 9×10⁻⁸ M or more, about 1×10⁻⁷ M or more, about 2×10⁻⁷ M or more, or about 5×10⁻⁷ M or more.

In certain embodiments, the antigen recognizing receptor is a chimeric co-stimulatory receptor (CCR). CCR is described in Krause, et al., J. Exp. Med. 188 (4): 619-626 (1998), and US20020018783, the contents of which are incorporated by reference in their entireties. CCRs mimic co-stimulatory signals, but unlike, CARs, do not provide a T-cell activation signal, e.g., CCRs lack a CD35 polypeptide. CCRs provide co-stimulation, e.g., a CD28-like signal, in the absence of the natural co-stimulatory ligand on the antigen-presenting cell. A combinatorial antigen recognition, i.e., use of a CCR in combination with a CAR, can augment T-cell reactivity against the dual-antigen expressing cells, thereby improving selective targeting. Kloss et al., describe a strategy that integrates combinatorial antigen recognition, split signaling, and, critically, balanced strength of T-cell activation and costimulation to generate T cells that eliminate target cells that express a combination of antigens while sparing cells that express each antigen individually (Kloss et al., Nature Biotechnology 31 (1): 71-75 (2013)). With this approach, T-cell activation requires CAR-mediated recognition of one antigen, whereas costimulation is independently mediated by a CCR specific for a second antigen. To achieve tumor selectivity, the combinatorial antigen recognition approach diminishes the efficiency of T-cell activation to a level where it is ineffective without rescue provided by simultaneous CCR recognition of the second antigen. In certain embodiments, the CCR comprises (a) an extracellular antigen-binding domain that binds to an antigen different than the first U5 snRNP200 antigen, (b) a transmembrane domain, and (c) a co-stimulatory signaling region that comprises at least one co-stimulatory molecule, including, but not limited to, CD28, 4-1BB, OX40, ICOS, PD-1, CTLA-4, LAG-3, 2B4, and BTLA. In certain embodiments, the co-stimulatory signaling region of the CCR comprises one co-stimulatory signaling molecule. In certain embodiments, the one co-stimulatory signaling molecule is CD28. In certain embodiments, the one co-stimulatory signaling molecule is 4-1BB. In certain embodiments, the co-stimulatory signaling region of the CCR comprises two co-stimulatory signaling molecules. In certain embodiments, the two co-stimulatory signaling molecules are CD28 and 4-1BB. A second antigen is selected so that expression of both the first U5 snRNP200 antigen and the second antigen is restricted to the targeted cells (e.g., cancerous cells). In some embodiments, the second antigen is CD19, CD22, CD20, CD21, CD23, CD72 and ROR1. Similar to a CAR, the extracellular antigen-binding domain of the CCR can be an scFv, a Fab, a F(ab)₂; or a fusion protein with a heterologous sequence to form the extracellular antigen-binding domain. In certain embodiments, the CCR comprises an scFv that binds to a B cell antigen (e.g., CD19, CD22, CD20, CD21, CD23, CD72 and ROR1), transmembrane domain comprising a CD28 polypeptide, and a co-stimulatory signaling region comprising two co-stimulatory signaling molecules that are CD28 and 4-1BB.

In certain embodiments, the antigen recognizing receptor is a truncated CAR. A “truncated CAR” is different from a CAR by lacking an intracellular signaling domain. For example, a truncated CAR comprises an extracellular antigen-binding domain and a transmembrane domain, and lacks an intracellular signaling domain. In accordance with the presently disclosed subject matter, the truncated CAR has a high binding affinity to the second antigen expressed on the targeted cells. The truncated CAR functions as an adhesion molecule that enhances the avidity of a presently disclosed CAR, especially, one that has a low binding affinity to a U5 snRNP200 antigen, thereby improving the efficacy of the presently disclosed CAR or engineered immune cell (e.g., T cell) comprising the same. In certain embodiments, the truncated CAR comprises an extracellular antigen-binding domain that binds to a B cell antigen (e.g., CD19, CD22, CD20, CD21, CD23, CD72 and ROR1), a transmembrane domain comprising a CD8 polypeptide. A presently disclosed T cell comprises or is transduced to express a presently disclosed CAR targeting U5 snRNP200 antigen and a truncated CAR targeting a B cell antigen (e.g., CD19, CD22, CD20, CD21, CD23, CD72 and ROR1). In certain embodiments, the targeted cells are AML or B cell malignancies. In some embodiments, the engineered immune cells are further modified to suppress expression of one or more genes. In some embodiments, the engineered immune cells are further modified via genome editing. Various methods and compositions for targeted cleavage of genomic DNA have been described. Such targeted cleavage events can be used, for example, to induce targeted mutagenesis, induce targeted deletions of cellular DNA sequences, and facilitate targeted recombination at a predetermined chromosomal locus. See, for example, U.S. Pat. Nos. 7,888,121; 7,972,854; 7,914,796; 7,951,925; 8,110,379; 8,409,861; 8,586,526; U.S. Patent Publications 20030232410; 20050208489; 20050026157; 20050064474; 20060063231; 201000218264; 20120017290; 20110265198; 20130137104; 20130122591; 20130177983 and 20130177960, the disclosures of which are incorporated by reference in their entireties. These methods often involve the use of engineered cleavage systems to induce a double strand break (DSB) or a nick in a target DNA sequence such that repair of the break by an error born process such as non-homologous end joining (NHEJ) or repair using a repair template (homology directed repair or HDR) can result in the knock out of a gene or the insertion of a sequence of interest (targeted integration). Cleavage can occur through the use of specific nucleases such as engineered zinc finger nucleases (ZFN), transcription-activator like effector nucleases (TALENs), or using the CRISPR/Cas system with an engineered crRNA/tracr RNA (‘single guide RNA’) to guide specific cleavage. In some embodiments, the engineered immune cells are modified to disrupt or reduce expression of an endogenous T-cell receptor gene (see, e.g., WO 2014153470, which is incorporated by reference in its entirety). In some embodiments, the engineered immune cells are modified to result in disruption or inhibition of PD1, PDL-1 or CTLA-4 (see, e.g., U.S. Patent Publication 20140120622), or other immunosuppressive factors known in the art (Wu et al. (2015) Oncoimmunology 4 (7): e1016700, Mahoney et al. (2015) Nature Reviews Drug Discovery 14, 561-584).

In any and all embodiments of the engineered immune cells disclosed herein, the engineered immune cells further comprise a construct encoding a secretable form of IL-18. Exemplary amino acid sequences and nucleic acid sequences of components of CAR constructs that secrete IL-18 are provided below:

MycTag_hCD28_hCD3Z_P2A_hIL18
MycTag
(SEQ ID NO: 72)
EQKLISEEDL
(SEQ ID NO: 73)
GAACAGAAACTGATCTCTGAAGAAGACCTG
hCD28 EC/TM/IC with preceding AAA ExtraCellular/TransMembrane/IntraCellular)
(SEQ ID NO: 74)
AAAIEVMYPPPYLDNEKSNGTIIHVKGKHLCPSPLFPGPSKPFWVLVVVGGVLACY
SLLVTVAFIIFWVRSKRSRLLHSDYMNMTPRRPGPTRKHYQPYAPPRDFAAYRS
(SEQ ID NO: 75)
GCGGCCGCAATTGAAGTTATGTATCCTCCTCCTTACCTAGACAATGAGAAGAGC
AATGGAACCATTATCCATGTGAAAGGGAAACACCTTTGTCCAAGTCCCCTATTT
CCCGGACCTTCTAAGCCCTTTTGGGTGCTGGTGGTGGTTGGTGGAGTCCTGGCTT
GCTATAGCTTGCTAGTAACAGTGGCCTTTATTATTTTCTGGGTGAGGAGTAAGA
GGAGCAGGCTCCTGCACAGTGACTACATGAACATGACTCCCCGCCGCCCCGGG
CCCACCCGCAAGCATTACCAGCCCTATGCCCCACCACGCGACTTCGCAGCCTAT
CGCTCC
hCD3Z IC
(SEQ ID NO: 57)
RVKFSRSADAPAYQQGQNQLYNELNLGRREEYDVLDKRRGRDPEMGGKPRRKNP
QEGLYNELQKDKMAEAYSEIGMKGERRRGKGHDGLYQGLSTATKDTYDALHMQ
ALPPR
(SEQ ID NO: 76)
AGAGTGAAGTTCAGCAGGAGCGCAGACGCCCCCGCGTACCAGCAGGGCCAGAA
CCAGCTCTATAACGAGCTCAATCTAGGACGAAGAGAGGAGTACGATGTTTTGG
ACAAGAGACGTGGCCGGGACCCTGAGATGGGGGGAAAGCCGAGAAGGAAGAA
CCCTCAGGAAGGCCTGTACAATGAACTGCAGAAAGATAAGATGGCGGAGGCCT
ACAGTGAGATTGGGATGAAAGGCGAGCGCCGGAGGGGCAAGGGGCACGATGG
CCTTTACCAGGGTCTCAGTACAGCCACCAAGGACACCTACGACGCCCTTCACAT
GCAGGCCCTGCCCCCTCGC
P2A with preceding GSG
(SEQ ID NO: 77)
GSGATNFSLLKQAGDVEENPGP
(SEQ ID NO: 78)
GGATCTGGAGCAACAAACTTCTCACTACTCAAACAAGCAGGTGACGTGGAGGA
GAATCCCGGACCC
IL-2 signal peptide
(SEQ ID NO: 79)
MGYRMQLLSCIALSLALVTNS
(SEQ ID NO: 80)
ATGGGTTACAGGATGCAACTCCTGTCTTGCATTGCACTAAGTCTTGCACTTGTCA
CAAACAGT
IL-18, human
(SEQ ID NO: 81)
GYFGKLESKLSVIRNLNDQVLFIDQGNRPLFEDMTDSDCRDNAPRTIFIISMYKDSQP
RGMAVTISVKCEKISTLSCENKIISFKEMNPPDNIKDTKSDIIFFQRSVPGHDNKMQF
ESSSYEGYFLACEKERDLFKLILKKEDELGDRSIMFTVQNED
(SEQ ID NO: 82)
GGCTACTTTGGCAAGCTTGAATCTAAATTATCAGTCATAAGAAATTTGAATGAC
CAAGTTCTCTTCATTGACCAAGGAAATCGGCCTCTATTTGAAGATATGACTGAT
TCTGACTGTAGAGATAATGCACCCCGGACCATATTTATTATAAGTATGTATAAA
GATAGCCAGCCTAGAGGTATGGCTGTAACTATCTCTGTGAAGTGTGAGAAAATT
TCAACTCTCTCCTGTGAGAACAAAATTATTTCCTTTAAGGAAATGAATCCTCCTG
ATAACATCAAGGATACAAAAAGTGACATCATATTCTTTCAGAGAAGTGTCCCAG
GACATGATAATAAGATGCAATTTGAATCTTCATCATACGAAGGATACTTTCTAG
CTTGTGAAAAAGAGAGAGACCTTTTTAAACTCATTTTGAAAAAAGAGGATGAA
TTGGGGGATAGATCTATAATGTTCACTGTTCAAAACGAAGAC
MycTag_hSAVY (YSNV)_hCD3Z_P2A hIL18
MycTag
(SEQ ID NO: 72)
EQKLISEEDL
(SEQ ID NO: 73)
GAACAGAAACTGATCTCTGAAGAAGACCTG
hCD28 with YSNV mutation (SAVY) EC/TM/IC with preceding AAA
(ExtraCellular/TransMembrane/IntraCellular)
(SEQ ID NO: 83)
AAAIEVMYPPPYLDNEKSNGTIIHVKGKHLCPSPLFPGPSKPFWVLVVVGGVLACY
SLLVTVAFIIFWVRSKRSRLLHSDYSNVTPRRPGPTRKHYQPYAPPRDFAAYRS
(SEQ ID NO: 84)
GCGGCCGCAATTGAAGTTATGTATCCTCCTCCTTACCTAGACAATGAGAAGAGC
AATGGAACCATTATCCATGTGAAAGGGAAACACCTTTGTCCAAGTCCCCTATTT
CCCGGACCTTCTAAGCCCTTTTGGGTGCTGGTGGTGGTTGGTGGAGTCCTGGCTT
GCTATAGCTTGCTAGTAACAGTGGCCTTTATTATTTTCTGGGTGAGGAGTAAGA
GGAGCAGGCTCCTGCACAGTGACTACTCAAATGTTACTCCCCGCCGCCCCGGGC
CCACCCGCAAGCATTACCAGCCCTATGCCCCACCACGCGACTTCGCAGCCTATC
GCTCC
hCD3Z IC
(SEQ ID NO: 57)
RVKFSRSADAPAYQQGQNQLYNELNLGRREEYDVLDKRRGRDPEMGGKPRRKNP
QEGLYNELQKDKMAEAYSEIGMKGERRRGKGHDGLYQGLSTATKDTYDALHMQ
ALPPR
(SEQ ID NO: 76)
AGAGTGAAGTTCAGCAGGAGCGCAGACGCCCCCGCGTACCAGCAGGGCCAGAA
CCAGCTCTATAACGAGCTCAATCTAGGACGAAGAGAGGAGTACGATGTTTTGG
ACAAGAGACGTGGCCGGGACCCTGAGATGGGGGGAAAGCCGAGAAGGAAGAA
CCCTCAGGAAGGCCTGTACAATGAACTGCAGAAAGATAAGATGGCGGAGGCCT
ACAGTGAGATTGGGATGAAAGGCGAGCGCCGGAGGGGCAAGGGGCACGATGG
CCTTTACCAGGGTCTCAGTACAGCCACCAAGGACACCTACGACGCCCTTCACAT
GCAGGCCCTGCCCCCTCGC
P2A with preceding GSG
(SEQ ID NO: 77)
GSGATNFSLLKQAGDVEENPGP
(SEQ ID NO: 78)
GGATCTGGAGCAACAAACTTCTCACTACTCAAACAAGCAGGTGACGTGGAGGA
GAATCCCGGACCC
IL-2 signal peptide
(SEQ ID NO: 79)
MGYRMQLLSCIALSLALVTNS
(SEQ ID NO: 80)
ATGGGTTACAGGATGCAACTCCTGTCTTGCATTGCACTAAGTCTTGCACTTGTCA
CAAACAGT
IL-18, human
(SEQ ID NO: 81)
GYFGKLESKLSVIRNLNDQVLFIDQGNRPLFEDMTDSDCRDNAPRTIFIISMYKDSQP
RGMAVTISVKCEKISTLSCENKIISFKEMNPPDNIKDTKSDIIFFQRSVPGHDNKMQF
ESSSYEGYFLACEKERDLFKLILKKEDELGDRSIMFTVQNED
(SEQ ID NO: 82)
GGCTACTTTGGCAAGCTTGAATCTAAATTATCAGTCATAAGAAATTTGAATGAC
CAAGTTCTCTTCATTGACCAAGGAAATCGGCCTCTATTTGAAGATATGACTGAT
TCTGACTGTAGAGATAATGCACCCCGGACCATATTTATTATAAGTATGTATAAA
GATAGCCAGCCTAGAGGTATGGCTGTAACTATCTCTGTGAAGTGTGAGAAAATT
TCAACTCTCTCCTGTGAGAACAAAATTATTTCCTTTAAGGAAATGAATCCTCCTG
ATAACATCAAGGATACAAAAAGTGACATCATATTCTTTCAGAGAAGTGTCCCAG
GACATGATAATAAGATGCAATTTGAATCTTCATCATACGAAGGATACTTTCTAG
CTTGTGAAAAAGAGAGAGACCTTTTTAAACTCATTTTGAAAAAAGAGGATGAA
TTGGGGGATAGATCTATAATGTTCACTGTTCAAAACGAAGAC

Additionally or alternatively, in some embodiments, the engineered immune cell of the present technology further comprises a signal peptide that is operably linked to the N-terminus of the IL-18 polypeptide. In certain embodiments, the signal peptide operably linked to the N-terminus of the IL-18 polypeptide comprises the amino acid sequence of SEQ ID NO: 79.

In any and all embodiments of the engineered immune cell described herein, the IL-18 polypeptide comprises the amino acid sequence of SEQ ID NO: 81. Additionally or alternatively, in some embodiments, the engineered immune cell of the present technology comprises a CD28 polypeptide comprising the amino acid sequence of SEQ ID NO: 74 or SEQ ID NO: 83.

### Polynucleotides, Polypeptides and Analogs

Also included in the presently disclosed subject matter are polypeptides including extracellular antigen-binding fragments that specifically bind to a U5 snRNP200 antigen (e.g., a human U5 snRNP200 antigen) (e.g., an scFv (e.g., a human scFv), a Fab, or a (Fab) 2), CD3, CD8, CD28, etc. or fragments thereof, and polynucleotides encoding the same, that are modified in ways that enhance their biological activity when expressed in an engineered immune cell. The presently disclosed subject matter provides methods for optimizing an amino acid sequence or a nucleic acid sequence by producing an alteration in the sequence. Such alterations may comprise certain mutations, deletions, insertions, or post-translational modifications. The presently disclosed subject matter further comprises analogs of any naturally-occurring polypeptide of the presently disclosed subject matter. Analogs can differ from a naturally-occurring polypeptide of the presently disclosed subject matter by amino acid sequence differences, by post-translational modifications, or by both. Analogs of the presently disclosed subject matter can generally exhibit at least about 85%, about 90%, about 91%, about 92%, about 93%, about 94%, about 95%, about 96%, about 97%, about 98%, about 99% or more identity or homology with all or part of a naturally-occurring amino acid sequence of the presently disclosed subject matter. The length of sequence comparison is at least about 5, about 10, about 15, about 20, about 25, about 50, about 75, about 100 or more amino acid residues. Again, in an exemplary approach to determining the degree of identity, a BLAST program may be used, with a probability score between e⁻³ and e⁻¹⁰⁰ indicating a closely related sequence. Modifications comprise in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring polypeptides of the presently disclosed subject matter by alterations in primary sequence. These include genetic variants, both natural and induced (for example, resulting from random mutagenesis by irradiation or exposure to ethanemethyl sulfate or by site-specific mutagenesis as described in Sambrook, Fritsch and Maniatis, Molecular Cloning: A Laboratory Manual (2nd ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., beta (β) or gamma (γ) amino acids.

In addition to full-length polypeptides, the presently disclosed subject matter also provides fragments of any one of the polypeptides or peptide domains of the presently disclosed subject matter. A fragment can be at least about 5, about 10, about 13, or about 15 amino acids. In some embodiments, a fragment is at least about 20 contiguous amino acids, at least about 30 contiguous amino acids, or at least about 50 contiguous amino acids. In some embodiments, a fragment is at least about 60 to about 80, about 100, about 200, about 300 or more contiguous amino acids. Fragments of the presently disclosed subject matter can be generated by methods known to those of ordinary skill in the art or may result from normal protein processing (e.g., removal of amino acids from the nascent polypeptide that are not required for biological activity or removal of amino acids by alternative mRNA splicing or alternative protein processing events).

Non-protein analogs have a chemical structure designed to mimic the functional activity of a protein of the present technology. Such analogs are administered according to methods of the presently disclosed subject matter. Such analogs may exceed the physiological activity of the original polypeptide. Methods of analog design are well known in the art, and synthesis of analogs can be carried out according to such methods by modifying the chemical structures such that the resultant analogs increase the antineoplastic activity of the original polypeptide when expressed in an engineered immune cell. These chemical modifications include, but are not limited to, substituting alternative R groups and varying the degree of saturation at specific carbon atoms of a reference polypeptide. The protein analogs can be relatively resistant to in vivo degradation, resulting in a more prolonged therapeutic effect upon administration. Assays for measuring functional activity include, but are not limited to, those described in the Examples below.

In accordance with the presently disclosed subject matter, the polynucleotides encoding an extracellular antigen-binding fragment that specifically binds to a U5 snRNP200 antigen (e.g., human U5 snRNP200 antigen) (e.g., an scFv (e.g., a human scFv), a Fab, or a (Fab) 2), CD3, CD8, CD28 can be modified by codon optimization. Codon optimization can alter both naturally occurring and recombinant gene sequences to achieve the highest possible levels of productivity in any given expression system. Factors that are involved in different stages of protein expression include codon adaptability, mRNA structure, and various cis-elements in transcription and translation. Any suitable codon optimization methods or technologies that are known to ones skilled in the art can be used to modify the polynucleotides of the presently disclosed subject matter, including, but not limited to, OptimumGene™, Encor optimization, and Blue Heron.

### Vectors

Many expression vectors are available and known to those of skill in the art and can be used for expression of polypeptides provided herein. The choice of expression vector will be influenced by the choice of host expression system. Such selection is well within the level of skill of the skilled artisan. In general, expression vectors can include transcriptional promoters and optionally enhancers, translational signals, and transcriptional and translational termination signals. Expression vectors that are used for stable transformation typically have a selectable marker which allows selection and maintenance of the transformed cells. In some cases, an origin of replication can be used to amplify the copy number of the vector in the cells.

Vectors also can contain additional nucleotide sequences operably linked to the ligated nucleic acid molecule, such as, for example, an epitope tag such as for localization, e.g., a hexa-his tag (SEQ ID NO: 85) or a myc tag, hemagglutinin tag or a tag for purification, for example, a GST fusion, and a sequence for directing protein secretion and/or membrane association.

Expression of antibodies or antigen binding fragments thereof can be controlled by any promoter/enhancer known in the art. Suitable bacterial promoters are well known in the art and described herein below. Other suitable promoters for mammalian cells, yeast cells and insect cells are well known in the art and some are exemplified below. Selection of the promoter used to direct expression of a heterologous nucleic acid depends on the particular application and is within the level of skill of the skilled artisan. Promoters which can be used include but are not limited to eukaryotic expression vectors containing the SV40 early promoter (Bernoist and Chambon, Nature 290:304-310 (1981)), the promoter contained in the 3′ long terminal repeat of Rous sarcoma virus (Yamamoto et al., Cell 22:787-797 (1980)), the herpes thymidine kinase promoter (Wagner et al., Proc. Natl. Acad. Sci. USA 75:1441-1445 (1981)), the regulatory sequences of the metallothionein gene (Brinster et al., Nature 296:39-42 (1982)); prokaryotic expression vectors such as the β-lactamase promoter (Jay et al., Proc. Natl. Acad. Sci. USA 75:5543 (1981)) or the tac promoter (DeBoer et al., Proc. Natl. Acad. Sci. USA 50:21-25 (1983)); see also “Useful Proteins from Recombinant Bacteria”: in Scientific American 242:79-94 (1980)); plant expression vectors containing the nopaline synthetase promoter (Herrera-Estrella et al., Nature 505:209-213 (1984)) or the cauliflower mosaic virus 35S RNA promoter (Gardner et al., Nucleic Acids Res. 9: 2871 (1981)), and the promoter of the photosynthetic enzyme ribulose bisphosphate carboxylase (Herrera-Estrella et al., Nature 510:1 15-120 (1984)); promoter elements from yeast and other fungi such as the Gal4 promoter, the alcohol dehydrogenase promoter, the phosphoglycerol kinase promoter, the alkaline phosphatase promoter, and the following animal transcriptional control regions that exhibit tissue specificity and have been used in transgenic animals: elastase I gene control region which is active in pancreatic acinar cells (Swift et al., Cell 55:639-646 (1984); Ornitz et al., Cold Spring Harbor Symp. Quant. Biol. 50:399-409 (1986); MacDonald, Hepatology 7:425-515 (1987)); insulin gene control region which is active in pancreatic beta cells (Hanahan et al., Nature 515:115-122 (1985)), immunoglobulin gene control region which is active in lymphoid cells (Grosschedl et al., Cell 55:647-658 (1984); Adams et al., Nature 515:533-538 (1985); Alexander et al., Mol. Cell Biol. 7:1436-1444 (1987)), mouse mammary tumor virus control region which is active in testicular, breast, lymphoid and mast cells (Leder et al., Cell 15:485-495 (1986)), albumin gene control region which is active in liver (Pinckert et al., Genes and Devel. 1:268-276 (1987)), alpha-fetoprotein gene control region which is active in liver (Krumlauf et al., Mol. Cell. Biol. 5:1639-403 (1985)); Hammer et al., Science 255:53-58 (1987)), alpha-1 antitrypsin gene control region which is active in liver (Kelsey et al., Genes and Devel. 7:161-171 (1987)), beta globin gene control region which is active in myeloid cells (Magram et al., Nature 515:338-340 (1985)); Kollias et al., Cell 5:89-94 (1986)), myelin basic protein gene control region which is active in oligodendrocyte cells of the brain (Readhead et al., Cell 15:703-712 (1987)), myosin light chain-2 gene control region which is active in skeletal muscle (Shani, Nature 514:283-286 (1985)), and gonadotrophic releasing hormone gene control region which is active in gonadotrophs of the hypothalamus (Mason et al., Science 254:1372-1378 (1986)).

In addition to the promoter, the expression vector typically contains a transcription unit or expression cassette that contains all the additional elements required for the expression of an antibody, or antigen binding fragment thereof, in host cells. A typical expression cassette contains a promoter operably linked to the nucleic acid sequence encoding the polypeptide chains of interest and signals required for efficient polyadenylation of the transcript, ribosome binding sites and translation termination. Additional elements of the cassette can include enhancers. In addition, the cassette typically contains a transcription termination region downstream of the structural gene to provide for efficient termination. The termination region can be obtained from the same gene as the promoter sequence or can be obtained from different genes.

Some expression systems have markers that provide gene amplification such as thymidine kinase and dihydrofolate reductase. Alternatively, high yield expression systems not involving gene amplification are also suitable, such as using a baculovirus vector in insect cells, with a nucleic acid sequence encoding a germline antibody chain under the direction of the polyhedron promoter or other strong baculovirus promoter.

Any methods known to those of skill in the art for the insertion of DNA fragments into a vector can be used to construct expression vectors containing a nucleic acid encoding any of the polypeptides provided herein. These methods can include in vitro recombinant DNA and synthetic techniques and in vivo recombinants (genetic recombination). The insertion into a cloning vector can, for example, be accomplished by ligating the DNA fragment into a cloning vector which has complementary cohesive termini. If the complementary restriction sites used to fragment the DNA are not present in the cloning vector, the ends of the DNA molecules can be enzymatically modified. Alternatively, any site desired can be produced by ligating nucleotide sequences (linkers) onto the DNA termini; these ligated linkers can contain specific chemically synthesized nucleic acids encoding restriction endonuclease recognition sequences.

Exemplary plasmid vectors useful to produce the polypeptides provided herein contain a strong promoter, such as the HCMV immediate early enhancer/promoter or the MHC class I promoter, an intron to enhance processing of the transcript, such as the HCMV immediate early gene intron A, and a polyadenylation (poly A) signal, such as the late SV40 polyA signal.

Genetic modification of engineered immune cells (e.g., T cells, NK cells) can be accomplished by transducing a substantially homogeneous cell composition with a recombinant DNA or RNA construct. The vector can be a retroviral vector (e.g., gamma retroviral), which is employed for the introduction of the DNA or RNA construct into the host cell genome. For example, a polynucleotide encoding the U5 snRNP200-specific receptor (e.g., a U5 snRNP200-specific chimeric antigen receptor) can be cloned into a retroviral vector and expression can be driven from its endogenous promoter, from the retroviral long terminal repeat, or from an alternative internal promoter.

Non-viral vectors or RNA may be used as well. Random chromosomal integration, or targeted integration (e.g., using a nuclease, transcription activator-like effector nucleases (TALENs), Zinc-finger nucleases (ZFNs), and/or clustered regularly interspaced short palindromic repeats (CRISPRs), or transgene expression (e.g., using a natural or chemically modified RNA) can be used.

For initial genetic modification of the cells to provide U5 snRNP200-specific receptor (e.g., a U5 snRNP200-specific chimeric antigen receptor) expressing cells, a retroviral vector is generally employed for transduction, however any other suitable viral vector or non-viral delivery system can be used. For subsequent genetic modification of the cells to provide cells comprising an antigen presenting complex comprising at least two co-stimulatory ligands, retroviral gene transfer (transduction) likewise proves effective. Combinations of retroviral vector and an appropriate packaging line are also suitable, where the capsid proteins will be functional for infecting human cells. Various amphotropic virus-producing cell lines are known, including, but not limited to, PA12 (Miller, et al., Mol. Cell. Biol. 5:431-437 (1985)); PA317 (Miller, et al., Mol. Cell. Biol. 6:2895-2902 (1986)); and CRIP (Danos, et al. Proc. Natl. Acad. Sci. USA 85:6460-6464 (1988)). Non-amphotropic particles are suitable too, e.g., particles pseudotyped with VSVG, RD114 or GALV envelope and any other known in the art.

Possible methods of transduction also include direct co-culture of the cells with producer cells, e.g., by the method of Bregni, et al., Blood 80:1418-1422 (1992), or culturing with viral supernatant alone or concentrated vector stocks with or without appropriate growth factors and polycations, e.g., by the method of Xu, et al., Exp. Hemat. 22:223-230 (1994); and Hughes, et al., J. Clin. Invest. 89:1817 (1992).

Transducing viral vectors can be used to express a co-stimulatory ligand and/or secretes a cytokine (e.g., 4-1BBL and/or IL-12) in an engineered immune cell. In some embodiments, the chosen vector exhibits high efficiency of infection and stable integration and expression (see, e.g., Cayouette et al., Human Gene Therapy 8:423-430 (1997); Kido et al., Current Eye Research 15:833-844 (1996); Bloomer et al., Journal of Virology 71:6641- 6649, 1997; Naldini et al., Science 272:263 267 (1996); and Miyoshi et al., Proc. Natl. Acad. Sci. U.S.A. 94:10319, (1997)). Other viral vectors that can be used include, for example, adenoviral, lentiviral, and adeno-associated viral vectors, vaccinia virus, a bovine papilloma virus, or a herpes virus, such as Epstein-Barr Virus (also see, for example, the vectors of Miller, Human Gene Therapy 15-14, (1990); Friedman, Science 244:1275-1281 (1989); Eglitis et al., BioTechniques 6:608-614, (1988); Tolstoshev et al., Current Opinion in Biotechnology 1:55-61 (1990); Sharp, The Lancet 337:1277-1278 (1991); Cornetta et al., Nucleic Acid Research and Molecular Biology 36:311-322 (1987); Anderson, Science 226:401-409 (1984); Moen, Blood Cells 17:407-416 (1991); Miller et al., Biotechnology 7:980-990 (1989); Le Gal La Salle et al., Science 259:988-990 (1993); and Johnson, Chest 107:77S-83S (1995)). Retroviral vectors are particularly well developed and have been used in clinical settings (Rosenberg et al., N. Engl. J. Med 323:370 (1990); Anderson et al., U.S. Pat. No. 5,399,346).

In certain non-limiting embodiments, the vector expressing a presently disclosed U5 snRNP200-specific receptor (e.g., a U5 snRNP200-specific chimeric antigen receptor) is a retroviral vector, e.g., an oncoretroviral vector. In some instances, the retroviral vector is a SFG retroviral vector or murine stem cell virus (MSCV) retroviral vector. In certain non-limiting embodiments, the vector expressing a presently disclosed U5 snRNP200-specific receptor (e.g., a U5 snRNP200-specific chimeric antigen receptor) is a lentiviral vector or a transposon vector.

Non-viral approaches can also be employed for the expression of a protein in a cell. For example, a nucleic acid molecule can be introduced into a cell by administering the nucleic acid in the presence of lipofection (Feigner et al., Proc. Nat'l. Acad. Sci. U.S.A. 84:7413, (1987); Ono et al., Neuroscience Letters 17:259 (1990); Brigham et al., Am. J. Med. Sci. 298:278, (1989); Staubinger et al., Methods in Enzymology 101:512 (1983)), asialoorosomucoid-polylysine conjugation (Wu et al., Journal of Biological Chemistry 263:14621 (1988); Wu et al., Journal of Biological Chemistry 264:16985 (1989)), or by micro-injection under surgical conditions (Wolff et al., Science 247:1465 (1990)). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g., Zinc finger nucleases, meganucleases, or TALE nucleases). Transient expression may be obtained by RNA electroporation.

cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g., the elongation factor 1a enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

The resulting cells can be grown under conditions similar to those for unmodified cells, whereby the modified cells can be expanded and used for a variety of purposes.

### Administration

Engineered immune cells expressing the U5 snRNP200-specific receptor (e.g., a U5 snRNP200-specific chimeric antigen receptor) of the presently disclosed subject matter can be provided systemically or directly to a subject for treating cancer, such as AML or B cell malignancies (e.g., Diffuse large B-cell lymphoma (DLBCL), B-cell acute lymphoblastic leukemia (B-ALL), Follicular lymphoma, Chronic lymphocytic leukemia (CLL)/small lymphocytic lymphoma (SLL), Mantle cell lymphoma (MCL), Extranodal marginal zone B-cell lymphoma, Nodal marginal zone B-cell lymphoma, Splenic marginal zone B-cell lymphoma, Burkitt lymphoma, Lymphoplasmacytic lymphoma (Waldenstrom macroglobulinemia), Hairy cell leukemia, Primary central nervous system (CNS) lymphoma) or lung cancer (e.g., non-small cell lung cancer). In certain embodiments, engineered immune cells are directly injected into an organ of interest. Additionally or alternatively, the engineered immune cells are provided indirectly to the organ of interest, for example, by administration into the circulatory system (e.g., the tumor vasculature) or into the tissue of interest. Expansion and differentiation agents can be provided prior to, during or after administration of cells and compositions to increase production of T cells in vitro or in vivo.

Engineered immune cells of the presently disclosed subject matter can be administered in any physiologically acceptable vehicle, systemically or regionally, normally intravascularly, intraperitoneally, intrathecally, or intrapleurally, although they may also be introduced into bone or other convenient site where the cells may find an appropriate site for regeneration and differentiation (e.g., thymus). In certain embodiments, at least 1×10⁵ cells can be administered, eventually reaching 1×10¹⁰ or more. In certain embodiments, at least 1×10⁶ cells can be administered. A cell population comprising engineered immune cells can comprise a purified population of cells. Those skilled in the art can readily determine the percentage of engineered immune cells in a cell population using various well-known methods, such as fluorescence activated cell sorting (FACS). The ranges of purity in cell populations comprising engineered immune cells can be from about 50% to about 55%, from about 55% to about 60%, about 60% to about 65%, from about 65% to about 70%, from about 70% to about 75%, from about 75% to about 80%, from about 80% to about 85%; from about 85% to about 90%, from about 90% to about 95%, or from about 95 to about 100%. Dosages can be readily adjusted by those skilled in the art (e.g., a decrease in purity may require an increase in dosage). The engineered immune cells can be introduced by injection, catheter, or the like. If desired, factors can also be included, including, but not limited to, interleukins, e.g., IL-2, IL-3, IL 6, IL-11, IL-7, IL-12, IL-15, IL-21, as well as the other interleukins, the colony stimulating factors, such as G-, M- and GM-CSF, interferons, e.g., γ-interferon.

In certain embodiments, compositions of the presently disclosed subject matter comprise pharmaceutical compositions comprising engineered immune cells expressing a U5 snRNP200-specific receptor (e.g., a U5 snRNP200-specific chimeric antigen receptor) with a pharmaceutically acceptable carrier. Administration can be autologous or non-autologous. For example, engineered immune cells expressing a U5 snRNP200-specific receptor (e.g., a U5 snRNP200-specific chimeric antigen receptor) and compositions comprising the same can be obtained from one subject, and administered to the same subject or a different, compatible subject. Peripheral blood derived T cells of the presently disclosed subject matter or their progeny (e.g., in vivo, ex vivo or in vitro derived) can be administered via localized injection, including catheter administration, systemic injection, localized injection, intravenous injection, or parenteral administration. When administering a pharmaceutical composition of the presently disclosed subject matter (e.g., a pharmaceutical composition comprising engineered immune cells expressing a U5 snRNP200-specific receptor (e.g., a U5 snRNP200-specific chimeric antigen receptor)), it can be formulated in a unit dosage injectable form (solution, suspension, emulsion).

### Formulations

Engineered immune cells expressing a U5 snRNP200-specific receptor (e.g., a U5 snRNP200-specific chimeric antigen receptor) and compositions comprising the same can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the compositions of the presently disclosed subject matter, e.g., a composition comprising engineered immune cells, in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the presently disclosed subject matter, however, any vehicle, diluent, or additive used would have to be compatible with the engineered immune cells of the presently disclosed subject matter.

The compositions can be isotonic, i.e., they can have the same osmotic pressure as blood and lacrimal fluid. The desired isotonicity of the compositions of the presently disclosed subject matter may be accomplished using sodium chloride, or other pharmaceutically acceptable agents such as dextrose, boric acid, sodium tartrate, propylene glycol or other inorganic or organic solutes. Sodium chloride is suitable particularly for buffers containing sodium ions.

Viscosity of the compositions, if desired, can be maintained at the selected level using a pharmaceutically acceptable thickening agent. Methylcellulose can be used because it is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Those skilled in the art will recognize that the components of the compositions should be selected to be chemically inert and will not affect the viability or efficacy of the engineered immune cells as described in the presently disclosed subject matter. This will present no problem to those skilled in chemical and pharmaceutical principles, or problems can be readily avoided by reference to standard texts or by simple experiments (not involving undue experimentation), from this disclosure and the documents cited herein.

One consideration concerning the therapeutic use of the engineered immune cells of the presently disclosed subject matter is the quantity of cells necessary to achieve an optimal effect. The quantity of cells to be administered will vary for the subject being treated. In certain embodiments, from about 10² to about 10¹², from about 10³ to about 10¹¹, from about 10⁴ to about 10¹⁰, from about 10⁵ to about 10⁹, or from about 10⁶ to about 10⁸ engineered immune cells of the presently disclosed subject matter are administered to a subject. More effective cells may be administered in even smaller numbers. In some embodiments, at least about 1×10⁸, about 2×10⁸, about 3×10⁸, about 4×10⁸, about 5×10⁸, about 1×10⁹, about 5×10⁹, about 1×10¹⁰, about 5×10¹⁰, about 1×10¹¹, about 5×10¹¹, about 1×10¹² or more engineered immune cells of the presently disclosed subject matter are administered to a human subject. The precise determination of what would be considered an effective dose may be based on factors individual to each subject, including their size, age, sex, weight, and condition of the particular subject. Dosages can be readily ascertained by those skilled in the art from this disclosure and the knowledge in the art. Generally, engineered immune cells are administered at doses that are nontoxic or tolerable to the patient.

The skilled artisan can readily determine the amount of cells and optional additives, vehicles, and/or carrier in compositions to be administered in methods of the presently disclosed subject matter. Typically, any additives (in addition to the active cell(s) and/or agent(s)) are present in an amount of from about 0.001% to about 50% by weight) solution in phosphate buffered saline, and the active ingredient is present in the order of micrograms to milligrams, such as from about 0.0001 wt % to about 5 wt %, from about 0.0001 wt % to about 1 wt %, from about 0.0001 wt % to about 0.05 wt %, from about 0.001 wt % to about 20 wt %, from about 0.01 wt % to about 10 wt %, or from about 0.05 wt % to about 5 wt %. For any composition to be administered to an animal or human, and for any particular method of administration, toxicity should be determined, such as by determining the lethal dose (LD) and LD50 in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

### Therapeutic Uses of the Engineered Immune Cells of the Present Technology

For treatment, the amount of the engineered immune cells provided herein administered is an amount effective in producing the desired effect, for example, treatment or amelioration of the effects of cancer such as AML or B cell malignancies (e.g., Diffuse large B-cell lymphoma (DLBCL), B-cell acute lymphoblastic leukemia (B-ALL), Follicular lymphoma, Chronic lymphocytic leukemia (CLL)/small lymphocytic lymphoma (SLL), Mantle cell lymphoma (MCL), Extranodal marginal zone B-cell lymphoma, Nodal marginal zone B-cell lymphoma, Splenic marginal zone B-cell lymphoma, Burkitt lymphoma, Lymphoplasmacytic lymphoma (Waldenstrom macroglobulinemia), Hairy cell leukemia, Primary central nervous system (CNS) lymphoma), lung cancer (e.g., non-small cell lung cancer), or one or more symptoms thereof. An effective amount can be provided in one or a series of administrations of the engineered immune cells provided herein. An effective amount can be provided in a bolus or by continuous perfusion. For adoptive immunotherapy using antigen-specific T cells, while cell doses in the range of about 10⁶ to about 10¹⁰ are typically infused, lower doses of the engineered immune cells may be administered, e.g., about 10⁴ to about 10⁸.

Upon administration of the engineered immune cells into the subject, the engineered immune cells are induced that are specifically directed against a U5 snRNP200 antigen. The engineered immune cells of the presently disclosed subject matter can be administered by any methods known in the art, including, but not limited to, pleural administration, intravenous administration, subcutaneous administration, intranodal administration, intratumoral administration, intrathecal administration, intrapleural administration, intraperitoneal administration, and direct administration to the thymus. In certain embodiments, the engineered immune cells and the compositions comprising the same are intravenously administered to the subject in need. Methods for administering cells for adoptive cell therapies, including, for example, donor lymphocyte infusion and engineered immune cell therapies, and regimens for administration are known in the art and can be employed for administration of the engineered immune cells provided herein.

The presently disclosed subject matter provides various methods of using the engineered immune cells (e.g., T cells) provided herein, expressing a U5 snRNP200-specific receptor (e.g., a CAR).

For example, the presently disclosed subject matter provides methods of reducing tumor burden in a subject. In one non-limiting example, the method of reducing tumor burden comprises administering an effective amount of the presently disclosed engineered immune cells to the subject and administering a suitable antibody targeted to the tumor, thereby inducing tumor cell death in the subject. In some embodiments, the engineered immune cells and the antibody are administered at different times. For example, in some embodiments, the engineered immune cells are administered and then the antibody is administered. In some embodiments, the antibody is administered 1 hour, 2 hours, 3 hours, 4 hours, 5 hours, 6 hours, 7 hours, 8 hours, 9 hours, 10 hours, 11 hours, 12 hours, 18 hours, 24 hours, 30 hours, 26 hours, 48 hours or more than 48 hours after the administration of the engineered immune cells of the present technology.

The presently disclosed subject matter provides various methods of using the engineered immune cells (e.g., T cells) provided herein, expressing a U5 snRNP200-specific receptor (e.g., a CAR) disclosed herein. In some embodiments, the receptor of the engineered immune cell is a CAR, caTCR, or eTCR. For example, the presently disclosed subject matter provides methods of reducing tumor burden in a subject. In one non-limiting example, the method of reducing tumor burden comprises administering an effective amount of the presently disclosed engineered immune cells to the subject, thereby inducing tumor cell death in the subject.

The presently disclosed engineered immune cells can reduce the number of tumor cells, reduce tumor size, and/or eradicate the tumor in the subject. In certain embodiments, the method of reducing tumor burden comprises administering an effective amount of engineered immune cells to the subject, thereby inducing tumor cell death in the subject. Non-limiting examples of suitable tumors include acute myeloid leukemia (AML), B cell malignancies (e.g., Diffuse large B-cell lymphoma (DLBCL), B-cell acute lymphoblastic leukemia (B-ALL), Follicular lymphoma, Chronic lymphocytic leukemia (CLL)/small lymphocytic lymphoma (SLL), Mantle cell lymphoma (MCL), Extranodal marginal zone B-cell lymphoma, Nodal marginal zone B-cell lymphoma, Splenic marginal zone B-cell lymphoma, Burkitt lymphoma, Lymphoplasmacytic lymphoma (Waldenstrom macroglobulinemia), Hairy cell leukemia, Primary central nervous system (CNS) lymphoma), lung cancer (e.g., non-small cell lung cancer), and metastases thereof. In some embodiments, the cancer is a relapsed or refractory cancer. In some embodiments, the cancer is resistant to one or more cancer therapies, e.g., one or more chemotherapeutic drugs.

The presently disclosed subject matter also provides methods of increasing or lengthening survival of a subject with cancer (e.g., a tumor). In one non-limiting example, the method of increasing or lengthening survival of a subject with cancer (e.g., a tumor) comprises administering an effective amount of the presently disclosed engineered immune cell to the subject, thereby increasing or lengthening survival of the subject. The presently disclosed subject matter further provides methods for treating or preventing cancer (e.g., a tumor) in a subject, comprising administering the presently disclosed engineered immune cells to the subject. Also provided herein are methods for treating of inhibiting tumor growth or metastasis in a subject comprising contacting a tumor cell with an effective amount of any of the engineered immune cells provided herein.

Cancers whose growth may be inhibited using the engineered immune cells of the presently disclosed subject matter include cancers typically responsive to immunotherapy. Non-limiting examples of cancers for treatment include acute myeloid leukemia (AML), B cell malignancies (e.g., Diffuse large B-cell lymphoma (DLBCL), B-cell acute lymphoblastic leukemia (B-ALL), Follicular lymphoma, Chronic lymphocytic leukemia (CLL)/small lymphocytic lymphoma (SLL), Mantle cell lymphoma (MCL), Extranodal marginal zone B-cell lymphoma, Nodal marginal zone B-cell lymphoma, Splenic marginal zone B-cell lymphoma, Burkitt lymphoma, Lymphoplasmacytic lymphoma (Waldenstrom macroglobulinemia), Hairy cell leukemia, Primary central nervous system (CNS) lymphoma), lung cancer (e.g., non-small cell lung cancer), and metastases thereof.

Additionally, the presently disclosed subject matter provides methods of increasing immune-activating cytokine production in response to a cancer cell in a subject in need thereof. In one non-limiting example, the method comprises administering the presently disclosed engineered immune cell to the subject. The immune-activating cytokine can be granulocyte macrophage colony stimulating factor (GM-CSF), IFNα, IFN-β, IFN-γ, TNF-α, IL-2, IL-3, IL-6, IL-11, IL-7, IL-12, IL-15, IL-21, interferon regulatory factor 7 (IRF7), and combinations thereof. In certain embodiments, the engineered immune cells including a U5 snRNP200 antigen-specific CAR of the presently disclosed subject matter increase the production of GM-CSF, IFN-γ, and/or TNF-α.

Suitable human subjects for therapy typically comprise two treatment groups that can be distinguished by clinical criteria. Subjects with “advanced disease” or “high tumor burden” are those who bear a clinically measurable tumor. A clinically measurable tumor is one that can be detected on the basis of tumor mass (e.g., by palpation, CAT scan, sonogram, mammogram or X-ray; positive biochemical or histopathologic markers on their own are insufficient to identify this population). A pharmaceutical composition embodied in the presently disclosed subject matter is administered to these subjects to elicit an anti-tumor response, with the objective of palliating their condition. Ideally, reduction in tumor mass occurs as a result, but any clinical improvement constitutes a benefit. Clinical improvement comprises decreased risk or rate of progression or reduction in pathological consequences of the tumor.

Another group of suitable subjects is known in the art as the “adjuvant group.” These are individuals who have had a history of neoplasia, but have been responsive to another mode of therapy. The prior therapy can have included, but is not restricted to, surgical resection, radiotherapy, and traditional chemotherapy. As a result, these individuals have no clinically measurable tumor. However, they are suspected of being at risk for progression of the disease, either near the original tumor site, or by metastases. This group can be further subdivided into high-risk and low-risk individuals. The subdivision is made on the basis of features observed before or after the initial treatment. These features are known in the clinical arts, and are suitably defined for each different neoplasia. Features typical of high-risk subgroups are those in which the tumor has invaded neighboring tissues, or who show involvement of lymph nodes. Another group has a genetic predisposition to neoplasia but has not yet evidenced clinical signs of neoplasia.

The subjects can have an advanced form of disease, in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

Further modification can be introduced to the U5 snRNP200-specific receptor (e.g., a U5 snRNP200-specific chimeric antigen receptor)-expressing engineered immune cells (e.g., T cells) to avert or minimize the risks of immunological complications (known as “malignant T-cell transformation”), e.g., graft versus-host disease (GvHD). Modification of the engineered immune cells can include engineering a suicide gene into the U5 snRNP200-specific receptor (e.g., a U5 snRNP200-specific chimeric antigen receptor)-expressing T cells. Suitable suicide genes include, but are not limited to, Herpes simplex virus thymidine kinase (hsv-tk), inducible Caspase 9 Suicide gene (iCasp-9), and a truncated human epidermal growth factor receptor (EGFRt) polypeptide. In certain embodiments, the suicide gene is an EGFRt polypeptide. The EGFRt polypeptide can enable T cell elimination by administering anti-EGFR monoclonal antibody (e.g., cetuximab). EGFRt can be covalently joined to the C-terminus of the intracellular domain of the U5 snRNP200-specific receptor (e.g., a U5 snRNP200-specific chimeric antigen receptor). The suicide gene can be included within the vector comprising nucleic acids encoding the presently disclosed U5 snRNP200-specific receptor (e.g., a U5 snRNP200-specific chimeric antigen receptor). The incorporation of a suicide gene into a presently disclosed U5 snRNP200-specific receptor (e.g., a U5 snRNP200-specific chimeric antigen receptor) gives an added level of safety with the ability to eliminate the majority of CAR T cells within a very short time period. A presently disclosed engineered immune cell (e.g., a T cell) incorporated with a suicide gene can be pre-emptively eliminated at a given time point post CAR T cell infusion, or eradicated at the earliest signs of toxicity.

### Combination Therapy

The compositions of the present technology may be employed in conjunction with other therapeutic agents useful in the treatment of cancers, such as AML and B cell malignancies (e.g., Diffuse large B-cell lymphoma (DLBCL), B-cell acute lymphoblastic leukemia (B-ALL), Follicular lymphoma, Chronic lymphocytic leukemia (CLL)/small lymphocytic lymphoma (SLL), Mantle cell lymphoma (MCL), Extranodal marginal zone B-cell lymphoma, Nodal marginal zone B-cell lymphoma, Splenic marginal zone B-cell lymphoma, Burkitt lymphoma, Lymphoplasmacytic lymphoma (Waldenstrom macroglobulinemia), Hairy cell leukemia, Primary central nervous system (CNS) lymphoma) or lung cancer (e.g., non-small cell lung cancer). For example, engineered immune cells expressing a receptor comprising an anti-U5 snRNP200 antigen binding fragment (e.g., scFv) disclosed herein may be separately, sequentially or simultaneously administered with at least one additional cancer therapy. In some embodiments, the receptor is a CAR, caTCR, or eTCR.

In some embodiments, the additional cancer therapy is selected from among a chemotherapy, a radiation therapy, an immunotherapy, a monoclonal antibody, an anti-cancer nucleic acid, an anti-cancer protein, an anti-cancer virus or microorganism, a cytokine, or any combination thereof.

Radiation therapy includes, but is not limited to, exposure to radiation, e.g., ionizing radiation, UV radiation, as known in the art. Exemplary dosages include, but are not limited to, a dose of ionizing radiation at a range from at least about 2 Gy to not more than about 10 Gy or a dose of ultraviolet radiation at a range from at least about 5 J/m2 to not more than about 50 J/m2, usually about 10 J/m2.

The methods of the present technology may further comprise sequentially, separately, or simultaneously administering to the subject at least one chemotherapeutic agent, optionally selected from the group consisting of cytarabine (cytosine arabinoside or ara-C), anthracycline drugs (such as daunorubicin (daunomycin) or idarubicin), cladribine (2-CdA), fludarabine, mitoxantrone, etoposide (VP-16), 6-thioguanine (6-TG), hydroxyurea, corticosteroid drugs (e.g., prednisone or dexamethasone), methotrexate (MTX), 6-mercaptopurine (6-MP), azacytidine, and decitabine.

The methods of the present technology may further comprise sequentially, separately, or simultaneously administering to the subject at least one targeted therapy selected from among FLT3 inhibitors (e.g., Midostaurin, Gilteritinib), IDH inhibitors (e.g., Ivosidenib, Olutasidenib, Enasidenib, Gemtuzumab ozogamicin), BCL-2 inhibitors (e.g., Venetoclax), Hedgehog pathway inhibitors (e.g., Glasdegib), Tafasitamab, blinatumomab, loncastuximab tesirine, Epratuzumab, Inotuzumab ozogamicin, and hypomethylating agents.

In some embodiments, the methods further comprise sequentially, separately, or simultaneously administering an immunotherapy to the subject. In some embodiments, the immunotherapy regulates immune checkpoints. In further embodiments, the immunotherapy comprises, or consists essentially of, or yet further consists of an immune checkpoint inhibitor, such as an Cytotoxic T-Lymphocyte Associated Protein 4 (CTLA4) inhibitor, or a Programmed Cell Death 1 (PD-1) inhibitor, or a Programmed Death Ligand 1 (PD-L1) inhibitor, BiTE/DART (e.g., Flotetuzumab, Vibecotamab), Antibody drug conjugates (e.g., IMGN632), Magrolimab, and MBG453+HMA.

In yet further embodiments, the immune checkpoint inhibitor comprises, or consists essentially of, or yet further consists of an antibody or an equivalent thereof recognizing and binding to an immune checkpoint protein, such as an antibody or an equivalent thereof recognizing and binding to CTLA4 (for example, Yervoy (ipilimumab), CP-675,206 (tremelimumab), AK104 (cadonilimab), or AGEN1884 (zalifrelimab)), or an antibody or an equivalent thereof recognizing and binding to PD-1 (for example, Keytruda (pembrolizumab), Opdivo (nivolumab), Libtayo (cemiplimab), Tyvyt (sintilimab), BGB-A317 (tislelizumab), JS001 (toripalimab), SHR1210 (camrelizumab), GB226 (geptanolimab), JS001 (toripalimab), AB122 (zimberelimab), AK105 (penpulimab), HLX10 (serplulimab), BCD-100 (prolgolimab), AGEN2034 (balstilimab), MGA012 (retifanlimab), AK104 (cadonilimab), HX008 (pucotenlimab), PF-06801591 (sasanlimab), JNJ-63723283 (cetrelimab), MGD013 (tebotelimab), CT-011 (pidilizumab), or Jemperli (dostarlimab)), or an antibody or an equivalent thereof recognizing and binding to PD-L1 (for example, Tecentriq (atezolizumab), Imfinzi (durvalumab), Bavencio (avelumab), CS1001 (sugemalimab), or KN035 (envafolimab)).

In some embodiments, the methods further comprise sequentially, separately, or simultaneously administering a cytokine to the subject. In some embodiments, the cytokine is administered prior to, during, or subsequent to administration of the one or more engineered immune cells. In some embodiments, the cytokine is selected from the group consisting of interferon α, interferon β, interferon γ, complement C5a, IL-2, TNFα, CD40L, IL12, IL-23, IL15, IL17, CCL1, CCL11, CCL12, CCL13, CCL14-1, CCL14-2, CCL14-3, CCL15-1, CCL15-2, CCL16, CCL17, CCL18, CCL19, CCL19, CCL2, CCL20, CCL21, CCL22, CCL23-1, CCL23-2, CCL24, CCL25-1, CCL25-2, CCL26, CCL27, CCL28, CCL3, CCL3L1, CCL4, CCL4L1, CCL5, CCL6, CCL7, CCL8, CCL9, CCR10, CCR2, CCR5, CCR6, CCR7, CCR8, CCRL1, CCRL2, CX3CL1, CX3CR, CXCL1, CXCL10, CXCL11, CXCL12, CXCL13, CXCL14, CXCL15, CXCL16, CXCL2, CXCL3, CXCL4, CXCL5, CXCL6, CXCL7, CXCL8, CXCL9, CXCL9, CXCR1, CXCR2, CXCR4, CXCR5, CXCR6, CXCR7 and XCL2.

### Kits

The presently disclosed subject matter provides kits for the treatment or prevention of a disease, such as AML and B cell malignancies (e.g., Diffuse large B-cell lymphoma (DLBCL), B-cell acute lymphoblastic leukemia (B-ALL), Follicular lymphoma, Chronic lymphocytic leukemia (CLL)/small lymphocytic lymphoma (SLL), Mantle cell lymphoma (MCL), Extranodal marginal zone B-cell lymphoma, Nodal marginal zone B-cell lymphoma, Splenic marginal zone B-cell lymphoma, Burkitt lymphoma, Lymphoplasmacytic lymphoma (Waldenstrom macroglobulinemia), Hairy cell leukemia, Primary central nervous system (CNS) lymphoma) or lung cancer (e.g., non-small cell lung cancer). In certain embodiments, the kit comprises a therapeutic or prophylactic composition containing an effective amount of a nucleic acid encoding a receptor comprising an anti-U5 snRNP200 antigen binding fragment (e.g., scFv) or an engineered immune cell expressing the receptor. In some embodiments, the receptor is a CAR, caTCR, or eTCR. In particular embodiments, the engineered immune cell further expresses at least one co-stimulatory ligand.

In one aspect, the kits of the present technology comprise a therapeutic or prophylactic composition including an effective amount of any of the engineered immune cells disclosed herein in unit dosage form. In some embodiments, the kit comprises a sterile container which contains a therapeutic or prophylactic composition; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired, the nucleic acid encoding a receptor comprising a U5 snRNP200 antigen binding fragment (e.g., scFv) can be provided together with instructions for manufacturing engineered immune cells and administering the engineered immune cells to a subject having or at risk of developing cancer, such as AML and B cell malignancies (e.g., Diffuse large B-cell lymphoma (DLBCL), B-cell acute lymphoblastic leukemia (B-ALL), Follicular lymphoma, Chronic lymphocytic leukemia (CLL)/small lymphocytic lymphoma (SLL), Mantle cell lymphoma (MCL), Extranodal marginal zone B-cell lymphoma, Nodal marginal zone B-cell lymphoma, Splenic marginal zone B-cell lymphoma, Burkitt lymphoma, Lymphoplasmacytic lymphoma (Waldenstrom macroglobulinemia), Hairy cell leukemia, Primary central nervous system (CNS) lymphoma) or lung cancer (e.g., non-small cell lung cancer). The instructions will generally include information about the use of the composition for the treatment or prevention of cancer such as AML and B cell malignancies (e.g., Diffuse large B-cell lymphoma (DLBCL), B-cell acute lymphoblastic leukemia (B-ALL), Follicular lymphoma, Chronic lymphocytic leukemia (CLL)/small lymphocytic lymphoma (SLL), Mantle cell lymphoma (MCL), Extranodal marginal zone B-cell lymphoma, Nodal marginal zone B-cell lymphoma, Splenic marginal zone B-cell lymphoma, Burkitt lymphoma, Lymphoplasmacytic lymphoma (Waldenstrom macroglobulinemia), Hairy cell leukemia, Primary central nervous system (CNS) lymphoma) or lung cancer (e.g., non-small cell lung cancer).

In other embodiments, the instructions include at least one of the following: description of the therapeutic agent; dosage schedule and administration for treatment or prevention of cancer such as AML and B cell malignancies (e.g., Diffuse large B-cell lymphoma (DLBCL), B-cell acute lymphoblastic leukemia (B-ALL), Follicular lymphoma, Chronic lymphocytic leukemia (CLL)/small lymphocytic lymphoma (SLL), Mantle cell lymphoma (MCL), Extranodal marginal zone B-cell lymphoma, Nodal marginal zone B-cell lymphoma, Splenic marginal zone B-cell lymphoma, Burkitt lymphoma, Lymphoplasmacytic lymphoma (Waldenstrom macroglobulinemia), Hairy cell leukemia, Primary central nervous system (CNS) lymphoma), lung cancer (e.g., non-small cell lung cancer), or symptoms thereof; precautions; warnings; indications; counter-indications; overdose information; adverse reactions; animal pharmacology; clinical studies; and/or references. The instructions may be printed directly on the container (when present), or as a label applied to the container, or as a separate sheet, pamphlet, card, or folder supplied in or with the container.

In some embodiments, the at least one engineered immune cell of the present technology binds to target cells that express U5 snRNP200 on the cell surface. The at least one engineered immune cell of the present technology may be provided in the form of a prefilled syringe or autoinjection pen containing a sterile, liquid formulation or lyophilized preparation (e.g., Kivitz et al., Clin. Ther. 28:1619-29 (2006)).

A device capable of delivering the kit components through an administrative route may be included. Examples of such devices include syringes (for parenteral administration).

The kit components may be packaged together or separated into two or more containers. In some embodiments, the containers may be vials that contain sterile, lyophilized formulations of engineered immune cell composition that are suitable for reconstitution. A kit may also contain one or more buffers suitable for reconstitution and/or dilution of other reagents. Other containers that may be used include, but are not limited to, a pouch, tray, box, tube, or the like. Kit components may be packaged and maintained sterilely within the containers.

Also provided herein are kits for use in the manufacture of an engineered immune cell that expresses a receptor comprising an anti-U5 snRNP200 antigen binding fragment (e.g., scFv). In certain embodiments, the kit comprises a vector comprising a nucleic acid encoding an engineered receptor receptor (e.g., CAR, caTCR, or eTCR) or other cell-surface ligand including an anti-U5 snRNP200 antigen binding fragment (e.g., scFv).

### EXAMPLES

The present technology is further illustrated by the following Examples, which should not be construed as limiting in any way.

### Example 1: Material and Methods

### Cell Lines.

293 Glv9-packaging cells were maintained in Dulbecco's modified Eagle's medium (DMEM) supplemented with 10% fetal bovine serum (FBS), non-essential amino acids (NEAA) (Atlanta Biological Flowery Branch), 2 mM L-glutamine (Invitrogen), and 1% penicillin/streptomycin (P/S) (Invitrogen). U937 human acute monocytic leukemia cell line, OCI-AML2 human acute myeloid leukemia (AML) cell line, OCI-AML3 human AML cell line, MOLM14 human AML cell lines, K562 chronic myeloid leukemia cell line, FARAGE diffuse large B-cell lymphoma (DLBCL) cell line, WSU-DLCL2 DLBCL cell line, SU-DHL-4 DLBCL cell line, NALM6 B acute lymphoblastic leukemia (B-ALL) cell line, CCRF-SB B-ALL cell line, TMD8 DLBCL cell line, KARPAS DLBCL cell line, and RS4; 11 B-ALL cell line were transduced with firefly luciferase-GFP virus. All tumor cell lines were maintained in RPMI-1640 supplemented with 10% FBS, NEAA, 10 mM HEPES (Invitrogen), 2 mM L-glutamine, 1% P/S, and 11 mM glucose (Invitrogen).

U937 CD32A knock out cells generated previously⁶ and CD32A re-expression was achieved via introduction of the full-length sequence cloned into the PiggyBac vector. Murine RN2 cells (MLL-AF9+NRAS^{G12D}) were generated as previously described⁷ and cultured in RPMI+10% fetal bovine serum+1% penicillin/streptomycin and passaged every 2-3 days to maintain a density of less than 1×10⁶ cells per mL.

### Animals

8-10 week-old CD45.1⁺ mice were purchased from Jackson Laboratory and maintained until age 12 weeks prior to use for transplant studies. The inv (3) (3q21q26) mouse strain⁸ (RBRC09508) was provided by RIKEN BRC through the National BioResource Project of the MEXT/AMED, Japan. Mx1-Cre Sf3bl^{K700E/WT} inv (3)(q21q26) CD45.2⁺ cells⁹ were serially transplanted in CD45.1⁺ mice. Mice were bred and maintained in individual ventilated cages and fed with autoclaved food and water at Memorial Sloan Kettering Animal Facility. All animal procedures were completed in accordance with the Guidelines for the Care and Use of Laboratory Animals and were approved by the Institutional Animal Care and Use Committees at MSKCC. All mouse experiments were performed in accordance with a protocol approved by the MSKCC Institutional Animal Care and Use Committees (13-04-003).

### Generation of Recombinant Fc Receptor Engineered Antibodies

Anti-snRNP200 (clone 23) heavy and light chain variable regions were obtained from the patent literature, and subsequently cloned into expression constructs for the various murine subclass variants (e.g., mIgG1, mIgG1-D265A, mIgG2b, mIgG2b, hIgG1) as previously⁵. Recombinant antibodies were generated by transient transfection of Expi293 cells with heavy and light chain expression plasmids using previously described protocols. Before transfection, plasmid sequences were validated by direct sequencing (Genewiz). Recombinant IgG antibodies were purified from cell-free supernatants by affinity purification using protein G or protein A sepharose beads (GE Healthcare). Purified proteins were dialysed in PBS, filter-sterilized (0.22 μm), and stored at 4° C.

### Genome-Scale CRISPR/Cas9 Screening

GFP⁺ lentivirus (U6 promoter driving expression of sgRNA and RPBSA promoter driving Puromycin and Zsgreen) carrying the genome-wide human Brunello library (77,441 sgRNAs targeting 19,114 genes, and 1000 non-targeting control sgRNAs) was produced in 293T cells. Virus titer was determined by measuring the percentage of puromycin resistant cells following transduction. A titer resulting in approximately 30% transduction efficiency (puromycin resistant) was used for the following experiments to ensure only 1 viral integration per cell. U937 and K562 cells expressing Cas9 were transduced with Brunello lentivirus and puromycin selection (8 μg/mL for U937 or 4 μg/ml for K562) was performed for 2 days prior to FACS (Aria, BD Bioscience) for GFP⁺ cells. After an additional 8 days in culture, GFP⁺ cells were stained with APC labeled U5 snRNP200 antibody and subjected to FACS where the bottom and top 10% U5 snRNP200 expressing cell populations were collected. Cell pellets from each population were lysed and genomic DNA was extracted using the QIAamp DNA Mini Kit (Qiagen) and quantified by Qubit (ThermoScientific). gRNAs amplicons were amplified by PCR using TaKaRa ExTaq® DNA Polymerase (Takara) to add Illumina sequencing adapters and multiplexing barcodes. Amplicons were quantified by Qubit and Bioanalyzer (Agilent), multiplexed, and sequenced on an Illumina NextSeq 500 to obtain 75b.p. single-end reads. Demultiplexed FASTQ files were trimmed from both the 5′ and 3′ end to remove sequencing adapter and sgRNA derived sequences using cutadapt (v2.5) to yield the 20 nucleotide sequence of the sgRNA using the following parameters: -g TGTGGAAAGGACGAAACACCG-a GTTTTAGAGCTAGAAATAGCAAG (SEQ ID NO: 71)—maximum-length 20. Next, the frequency of each sgRNA was determined using the ‘count’ function of the MAGeCK (v0.5.9.4) software package. sgRNA counts were normalized to sequencing depth by applying the following parameters: —norm-method total. The corresponding normalized counts matrix was used to perform the indicated pairwise statistical comparisons using the ‘test’ function of the MAGeCK package. All visualization of these CRISPR/Cas9 screening data was performed in Rstudio (v1.3.1073) using the ggplot2 (v3.3.5) package. Function and pathway enrichment against existing gene ontology (GO) signatures was performed using ToppFun, part of the ToppGene Suite, and included GO terms and pathways from the KEGG, Reactome, and BioCarta databases. GO term or pathway was identified as significant under a Benjamini-Hochberg multiple correction procedure at a FDR cut-off of 0.05. As input for ToppFun, the top 150 enriched genes (ranked by FDR) were manually selected as identified by MAGeCK. GO term data were plotted in GraphPad Prism 8.

### Western Blotting

K562 expressing HaloTagged U5 snRNP200 cells (Synthego) were collected by centrifugation and subcellular fractions were obtained using a sub-cellular protein fractionation kit (ThermoFischer). Protein concentrations were measured with the BCA reagent and 10 mcg was loaded per lane onto 4-12% bis-tris protein gels. After transfer, PVDF membranes were probed with anti-snRNP200 (Bethyl Laboratories), anti-HaloTag antibody (Promega), sodium-potassium ATPase (Cell Signaling Technologies), tubulin (Cell Signaling Technologies), or SP1 (Cell Signaling Technologies) followed by appropriate peroxidase conjugated secondary antibodies and visualized as previously described.

### Flow Cytometry

For conventional flow cytometry experiments, cells were collected by centrifugation and washed once in cold PBS prior to application of Fixable LIVE/DEAD NIR (ThermoFischer). Mouse or human Fc receptor blocking was applied prior to staining (for all experiments where Fc receptors were not profiled individually) with antibody cocktail containing Brilliant Stain Buffer (BD Bioscience) and monocyte blocker (Biolegend). After cocktail staining, cells were washed twice with cold PBS prior to data acquisition using the Attune cytometer (ThermoFischer). The 36 color/parameter spectral flow cytometry panel was developed with guidance from Cytek Biosciences and recently published 40 color peripheral blood spectral flow cytometry panel (OMIP6910), including individual antibody titrations and comparison of performance in single versus multicolor staining. For the spectral flow cytometry experiments, human bone marrow samples were thawed using pre-warmed BD BSA stain buffer (BD Bioscience), washed twice with ice cold buffer, and counted. After application of Fixable LIVE/DEAD NIR (diluted 1:3000 in PBS; ThermoFischer) to a maximum of 5 million cells, sequential staining included Fc receptor antibody cocktail containing Brilliant Stain Buffer and monocyte blocker followed by CXCR5 antibody, and finally the remaining surface antibody panel cocktail. Prior to acquisition using the Aurora cytometer (Cytek), samples were washed twice in cold BD BSA stain buffer. Post-acquisition unmixing was performed using SpectroFlow Software version 3.0 (Cytek). Analysis of samples including scaling, data clean up gating, manual gating, UMAP generation, and heatmap generation was conducted using OMIQ software (online platform).

### Generation of Retroviral Constructs

CAR constructs were cloned into the SFG gammaretroviral vector with human signaling domains. Gpg29 fibroblasts (H29) were transfected with retroviral constructs encoding the CAR using CaPO4 (Promega) according to the manufacturer's instructions to generate retroviral producer cell lines. 293Glv9 cells were then transduced with retroviral supernatant from the H29 cells to produce stable retroviral producer cell lines.

### Bone Marrow (BM) Transplantation

Freshly dissected femora and tibiae were isolated from CD45.1⁺WT and CD45.2⁺ Mx1-Cre Sf3b1^{K700E/WT} inv (3)(q21q26) mice. The bones were spun at 1500 rpm by benchtop centrifugation and RBCs were lysed in ammonium chloride-potassium bicarbonate lysis buffer for 5 min. After centrifugation, cells were resuspended in ice cold sterile PBS, passed through a 100 μm cell strainer, and counted. Finally, 0.5 million total BM cells from CD45.2⁺ Mx1-Cre Sf3b1^{K700E/WT} inv (3)(q21q26) mice were mixed with 0.5 million WT CD45.1⁺ support BM and transplanted via tail vein injection into lethally irradiated (two times 450 cGy) CD45.1⁺ recipient mice. Engraftment was measured by flow cytometry from the peripheral blood 10 days after transplant. For syngeneic RN2 cell transplantation experiments, 50,000 cells were injected into sub-lethally irradiated (550 cGy) CD45.1⁺ recipient mice.

### Animal Antibody Treatments

Mice engrafted with RN2 or EVI1-rearranged Mx1-cre Sf3b1^{K700E/WT} AML were treated with 400 ul intraperitoneal (IP) injection of control (PBS) or antibody (1 mg/ml) as per indicated experiment schemas. Azacitidine was dissolved in 20% 2-Hydroxypropyl-β-cyclodextrin in sterile PBS and was dosed for 3 days (d1-3, RN2 model) or 5 days (d10-14, EVI1 model) at 3 mg/kg via IP injection. All whole-body bioluminescent imaging was performed by IP injection of Luciferin (Goldbio) at a 50 mg/kg concentration and imaging was performed after 5 minute incubation via IVIS. Bioluminescent signals (radiance) were quantified using Living Image software with standard regions of interests (ROI) rectangles.

### T Cell Isolation

Human T cells were derived from fresh blood-derived leukocyte concentrate (Leukopack) obtained from the New York Blood Center. Cell suspensions were lysed with ACK (Ammonium-Chloride-Potassium) Lysing Buffer (Lonza) and then mononuclear cells were separated by density gradient centrifugation with Accu-Prep cell separation media (Accurate Chemical & Scientific Corporation). T cells were isolated using EasySep Human T Cell Isolation Kit (STEMCELL Technologies) and were subsequently activated with 100 IU/mL recombinant human IL-2 (Prometheus Lab) and Dynabeads Human T-Activator CD3/CD28 (ThermoFisher Scientific) at a bead: cell ratio of 1:5. Purified T cells were cultured in RPMI 1640 with 100 IU/mL recombinant human IL-2.

### Retroviral Transduction

Retroviral supernatant collected from 293Glv9 packaging cells was loaded onto retronectin (Takara Bio)-coated plates. Activated T cells were spinoculated with retroviral supernatant and were subsequently transduced for 72 hours. CAR expression on the surface of T cells was detected using anti-Myc antibodies (Cell signaling Technology, #3739). Transduction efficiency of the CAR construct was determined by mCherry positivity.

### Quantitative Cytotoxicity Assay

CAR T cells were cocultured with 10,000 tumor cells expressing firefly luciferase at the different effector-to-target ratios in 96 well plates (Greiner Bio-One). 24 hours later, 15 μg D-Luciferin (Gold Biotechnology) dissolved in 50 μl of PBS was added to each well. Emitted luminescence of each sample was detected in a Spark plate reader (Tecan) and measured using SparkControl software (Tecan).

### Live Cell Imaging

CAR T cells were cocultured with 20,000 tumor cells expressing enhanced GFP at the different effector-to-target ratios in 96 well plates (Greiner Bio-One). mCherry positive CAR T cells and GFP positive tumor cells were enumerated every 8 hours with the Incucyte S3 Live-Cell Analysis Instrument (Sartorius).

### Intracellular Staining

CAR T cells were fixed with Cytofix/Cytoperm solution (BD Biosciences) for 10 min at room temperature and permeabilized with 90% methanol for 30 min at 4° C. Cells were then incubated with fluorochrome-conjugated anti-phospho-AKT1/2/3 (R&D Systems, IC7794P), anti-phospho-ERK1/2 (Invitrogen, 46-9109-42), and anti-phospho-ZAP70 (Invitrogen, 25-9006-42) antibodies for 30 min at 4° C.

### Cytokine Secretion Analysis

50,000 CAR T cells were cocultured with 50,000 tumor cells in 96 well plates. 24 hours later, the supernatant of culture media was collected to be analyzed for cytokine secretion analysis with Luminex IS100 instrument. Luminex FlexMap3D system, Luminex xPONENT 4.2, and 12-plex Human panel (Millipore) were used to detect cytokines. Heatmaps were generated by “ComplexHeatmap” package in the R software.

### In Vivo Bioluminescence Imaging

5×10⁴ U937 cells expressing firefly luciferase and 1×10⁶ U5 snRNP200 CAR T cells were transplanted into NCG mice on day 0 and day 3, respectively. Tumor burden was measured weekly (day 7, 14, 21, 28, and 35) by in vivo bioluminescence imaging using the Xenogen IVIS Imaging system (Xenogen) and analyzed on Living Image software (Xenogen).

### Example 2: Differential U5 snRNP200 Expression on AML Cells Versus Normal Hematopoietic Precursors

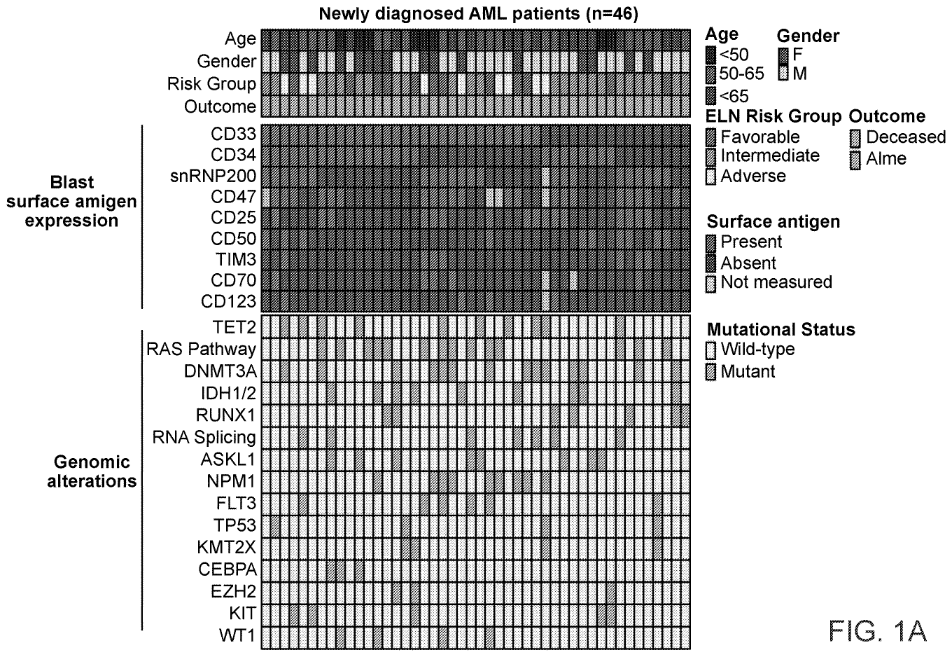

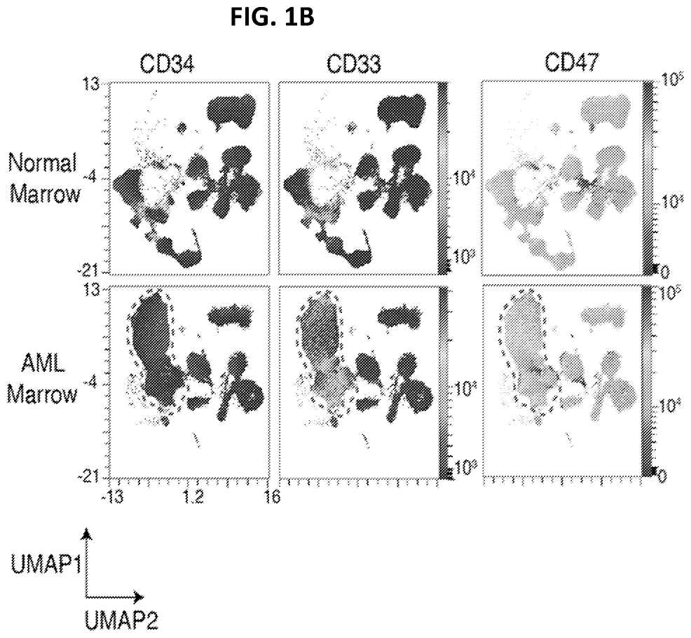

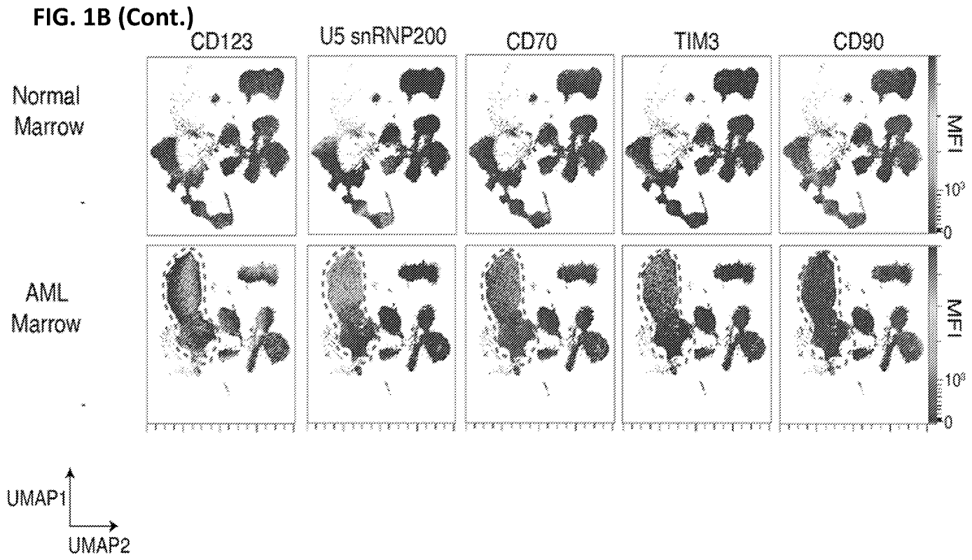

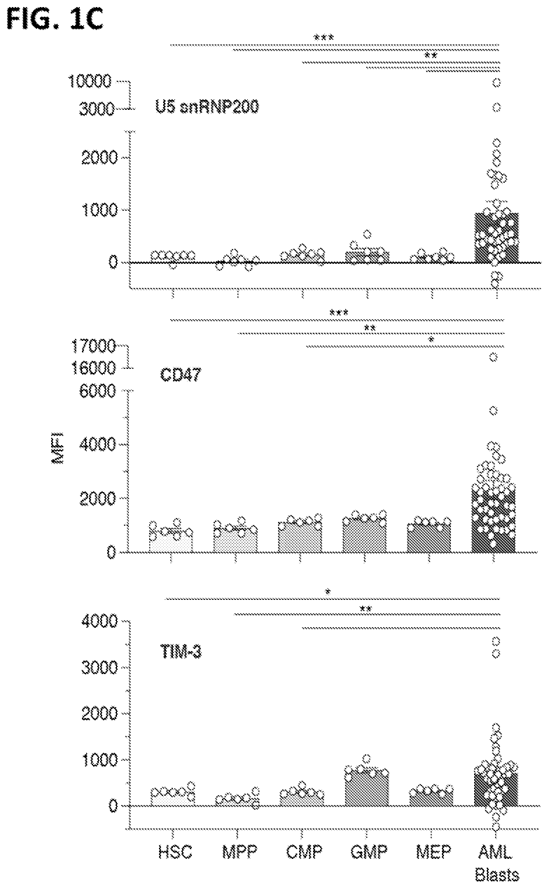

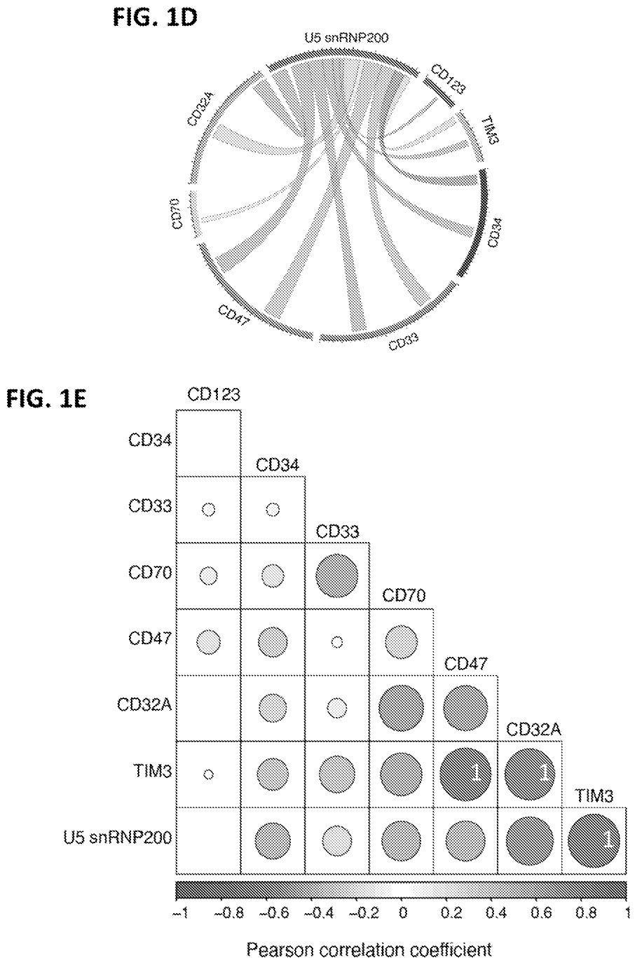

A custom 36-parameter spectral flow cytometry panel optimized to simultaneously interrogate AML blast surface phenotype, normal HSPC subsets, mature immune cells, and individual activating and inhibitory Fc receptors in human bone marrow was developed. This assay included assessment of antigens (CD123, TIM3, CD33, CD47, CD90, CD38, CD25, CD70, and U5 snRNP200) being actively assessed as potential AML cell surface targets in clinical trials or previously described as putative AML-associated antigens.¹⁻³ This panel was applied to bone marrow samples from 46 newly diagnosed clinically and genetically annotated adult AML patients. This cohort represents the heterogeneous range of disease features in newly diagnosed AML patients with a median age of 58 years and with 20% and 65% of patients being of intermediate or adverse risk, respectively, according to 2022 European LeukemiaNet risk classification (FIG. 1A).

Using live cell populations from bone marrow samples from 6 normal donors (median age 41.5) and AML patients as input, UMAP projections were generated to objectively delineate the malignant blast compartment from normal cell populations in an unbiased manner (FIG. 1B). One of the main challenges of current AML antibody-based therapeutics is on-target off-tumor side effects due to expression of the antibody target on normal HSPCs. Comparison of surface expression of antigens under evaluation for AML therapeutic targeting revealed increased abundance of CD47 (p=0.0005), TIM-3 (p=0.028), and U5 snRNP200 (p=0.0006) on the surface of AML cells relative to normal HSCs (Lin⁻CD34⁺CD45^{dim}CD90⁺CD38⁻) from age-matched healthy subjects consistent with prior reports (FIG. 1C). At the same time, the most significant differentially expressed antigen between AML blasts and normal CD34⁺ hematopoietic precursors was U5 snRNP200 as this antigen (originally identified as a potential AML-specific antigen by Gillissen et al.) was totally absent from normal HSCs, multipotent progenitors, and any downstream myeloid progenitor population (FIG. 1C). Of note, U5 snRNP200 was present on blasts from 50% of newly diagnosed AML patients.

Co-expression of antigens on AML blasts was also analyzed with the aim of defining antigen combinations suitable for multi- or bi-specific antibody or multi-antigen CAR-T therapy, approaches being actively pursued in hopes of reducing on-target off-tumor side effects. Indeed, several patterns of co-expression of antigens on AML blasts (FIGS. 1D-1E) were observed including statistically significant co-expression of U5 snRNP200 with CD47 (p=0.002) and TIM3 (p<0.0001), two antigens under evaluation using separate therapeutic antibodies in phase 2/3 clinical trials for patients with AML/MDS currently.

### Example 3: Localization and Surface Membrane Regulation of U5 snRNP200 Expression in AML

U5 snRNP200 is an ATP-dependent RNA helicase 250 kDa in size which is an essential, evolutionarily conserved core component of the spliceosome.⁴ Its function and molecular mechanism have been exquisitely defined as serving to unwind the duplex RNAs formed by the U4 and U6 small nuclear RNAs required for formation of the catalytic spliceosome. It was therefore unexpected that a nuclear enzyme involved in RNA splicing would be present on the cell membrane.

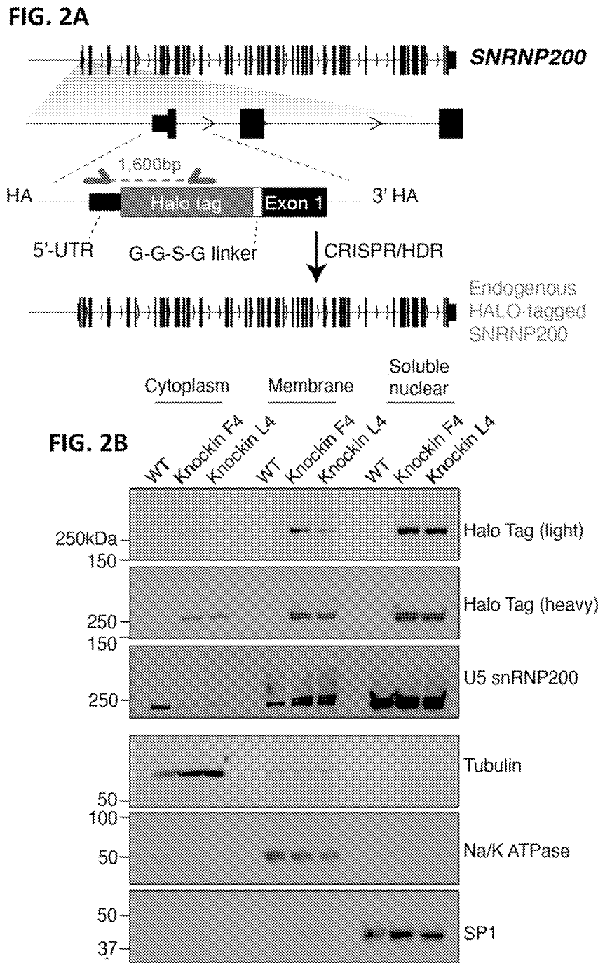

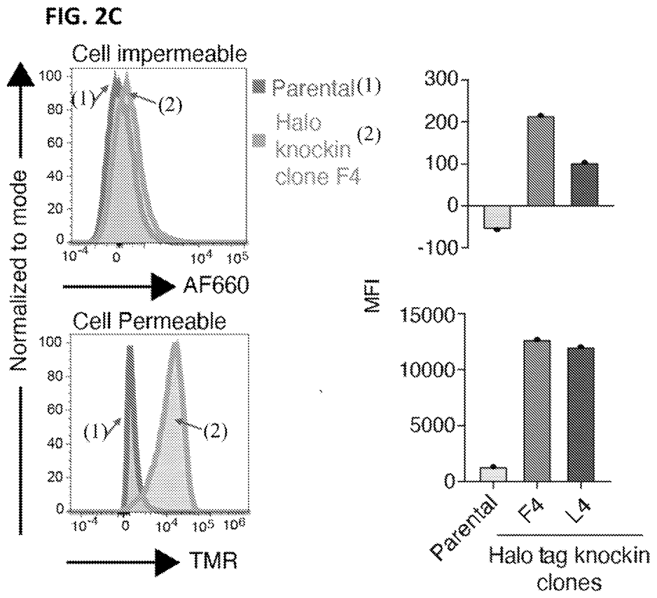

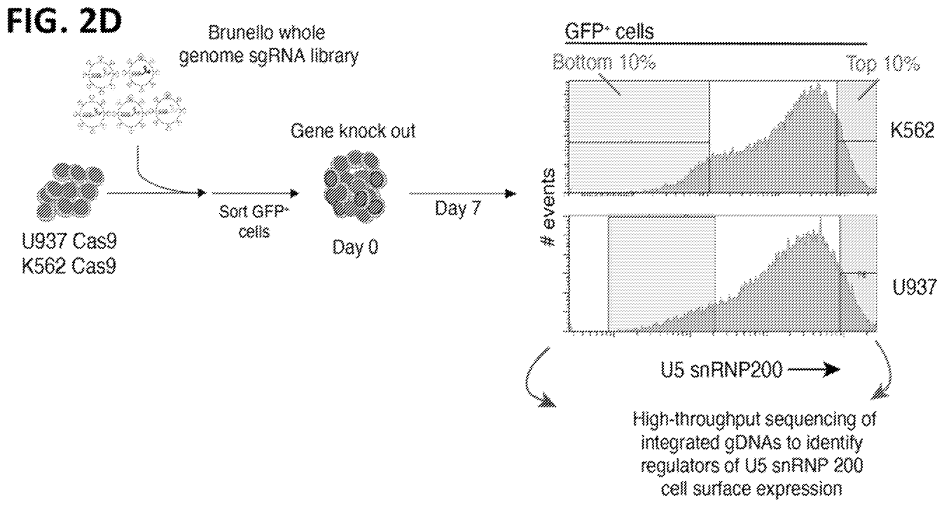

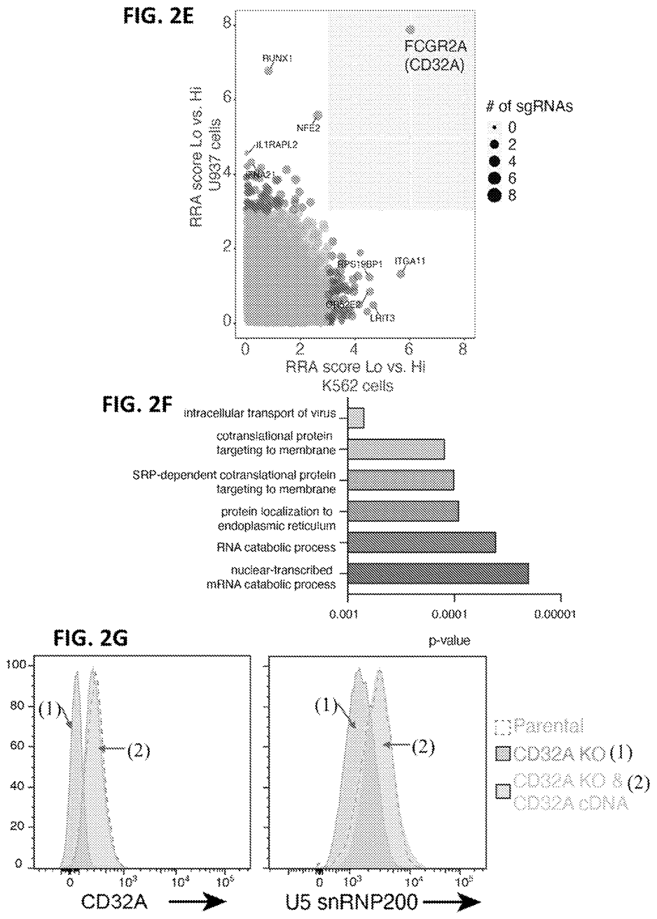

Given that antibody-based detection of the U5 snRNP200 alone may not reliably prove presence of full length U5 snRNP200 on the plasma membrane, this observation was validated by introducing the sequence encoding a HaloTag epitope in-frame into the N-terminus of SNRNP 200 in K562 human AML cells using CRISPR-mediated HDR editing (FIG. 2A). Sub-cellular fractionation of HaloTag knockin K562 cell clones and controls followed by Western blotting for HaloTag and U5 snRNP200 confirmed the presence of endogenous U5 snRNP200 in the nuclear fraction in all cells and at its full size of 250 kDa in the two HaloTag knockin clones. Moreover, Western blotting of lysates from distinct cellular compartments revealed localization of the full-length U5 snRNP200 (as indicated by the HaloTag) on the cell membrane (FIG. 2B). Localization of endogenous U5 snRNP200 at the cell membrane was further confirmed by utilizing cell impermeable fluorescent ligands that interact with the HaloTag (FIG. 2C). At the same time, the abundance of cell membrane localized U5 snRNP200 was only a fraction of U5 snRNP200 present within the cell as revealed by membrane permeable fluorescent ligands that interact with the HaloTag (FIG. 2C).

Given the unexpected presence of surface-membrane localized U5 snRNP200, the molecular regulators of surface U5 snRNP200 expression were investigated. To this end, the genome wide Brunello sgRNA library was applied to two human AML cell lines expressing cell surface U5 snRNP200 (K562 and U937 cells) and subsequently sorted the highest (top 10%) and lowest (bottom 10%) surface U5 snRNP200 expressing populations (FIG. 2D). Interestingly, sequencing of sgRNAs in these two populations revealed that knockout of FCGR2A, the gene that encodes the activating Fc receptor CD32A, was highly associated with loss of surface U5 snRNP200 expression in both cell lines (p<0.05; FIG. 2E). Moreover, gene ontology analysis indicated numerous genes encoding proteins required for subcellular protein trafficking that were also required for cell surface U5 snRNP200 expression (FIG. 2F).

To confirm the role of CD32A in surface U5 snRNP200 expression, flow cytometry was performed using U937 cells with CRISPR-mediated stable knockout of CD32A using sgRNA independent from those used in the CRISPR screen. Flow cytometric staining of CD32A confirmed diminished expression in CD32A knockout cells as expected (FIG. 2G). Importantly, U5 snRNP200 cell surface expression mirrored that of CD32A as U5 snRNP200 cell surface abundance was also abolished with CD32A KO. Moreover, restoration of surface CD32A expression using CD32A cDNA impervious to sgRNA knockout rescued both cell surface U5 snRNP200 and CD32A expression (FIG. 2G). Collectively, these studies rigorously validate surface U5 snRNP200 expression on AML cells and elucidate CD32A as a key regulator of this phenomenon.

### Example 4: In Vivo Efficacy of Anti-U5 snRNP200 Antibodies in Syngeneic Immunocompetent AML Models

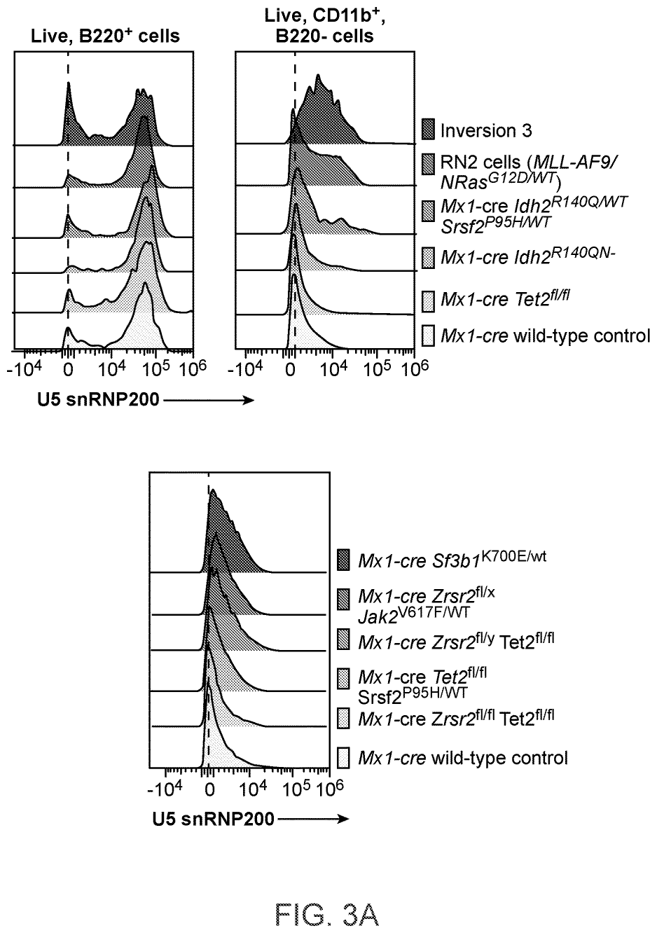

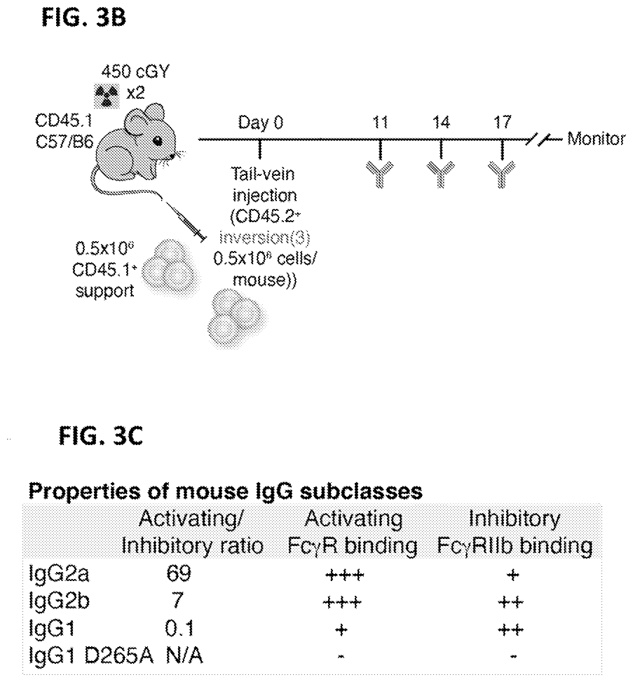

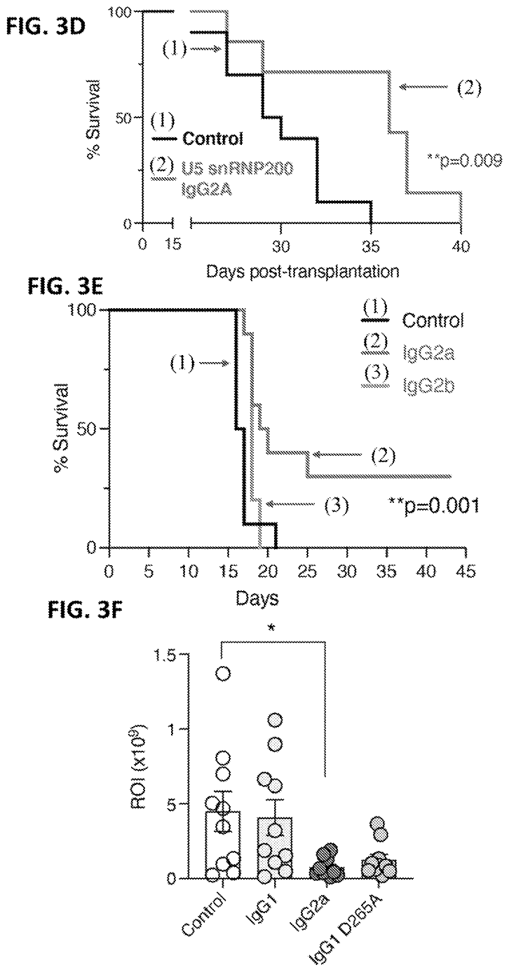

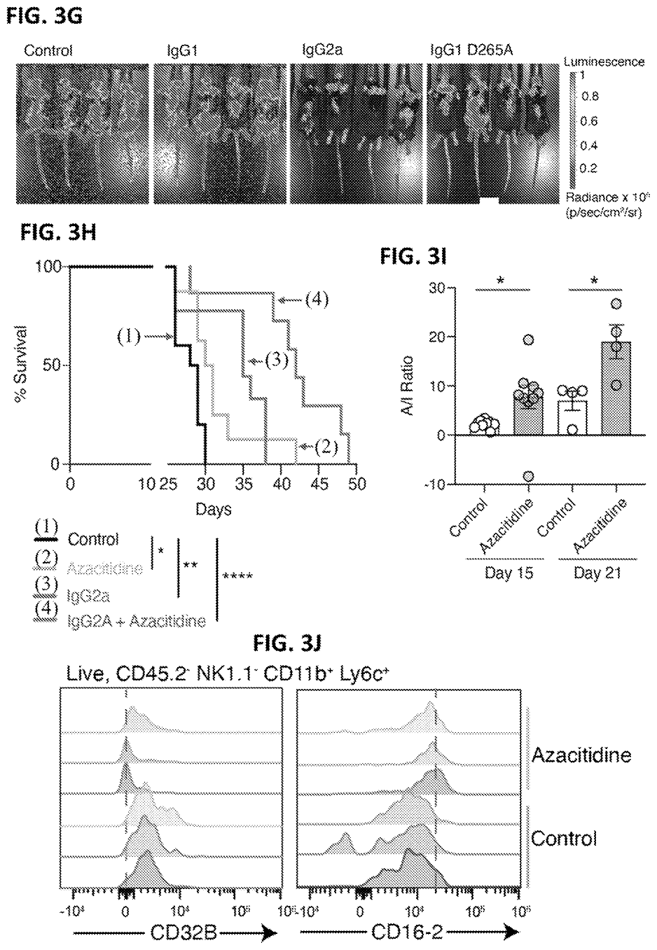

The presence of U5 snRNP200 on the surface of AML cells and not on normal HSPCs highlights U5 snRNP200 as an attractive therapeutic target in AML. To investigate the anti-leukemic effects of U5 snRNP200 antibodies in syngeneic immunocompetent AML models, surface U5 snRNP200 expression was first assessed in murine models of AML and control wild-type C57/B6 mice. While consistent expression of U5 snRNP200 surface expression on B220⁺ B-lymphocytes (FIG. 3A) was observed as seen in humans, a range of U5 snRNP200 surface expression on malignant myeloid cells was observed across a number of genetically engineered myeloid leukemia mouse models (FIG. 3A). Across 9 models, expression of U5 snRNP200 was most prominent on AML cells from mice bearing the humanized inversion chromosome 3q21q26 allele (“inversion 3 mice”) as well as simultaneous overexpression of MLL-AF9 and NRAS^{G12D} CDNAs (so called “RN2” cells).

Using the inversion 3 mouse AML model (FIG. 3B), an anti-U5 snRNP200 antibody featuring a murine Fc region optimized to engage activating Fc receptors on immune cell subsets that also has reduced binding to the inhibitory receptor FcRIIB (IgG2a; FIG. 3C) was tested. Single agent treatment with the IgG2a anti-U5 snRNP200 antibody yielded robust anti-leukemic activity leading to a survival benefit compared to control PBS treated mice (FIG. 3D). The fact that IgG2a U5 snRNP200 antibody provided therapeutic benefit suggests that the cellular mechanism of action of these antibodies may be via ADCC or ADCP. To test this hypothesis, U5 snRNP200 antibodies with murine subclass variant Fc regions that have lower activating: inhibitory ratios for Fc receptor engagement compared to the IgG2a variant were generated (FIG. 3C). This included generation of anti-U5 snRNP200 antibodies with murine IgG2b or IgG1 Fc regions as well as a engineered version of IgG1 with a D265A substitution which does not bind Fc receptors.⁵ As observed in the inversion 3 mouse model, in vivo treatment of animals engrafted with RN2 cells with the IgG2a variant antibody yielded a consistent survival benefit compared to the antibody variant IgG2b that is not optimized for binding to activating Fc receptors (FIG. 3E). Therapeutic benefit was also evaluated based on quantification of bioluminescent imaging (as RN2 cells also contain a luciferase vector) (FIGS. 3F-3G), which again supported IgG2a as the variant with maximum anti-leukemic effect as it provided significant disease control compared to variants that do not activate FcRs (IgG1 and IgG1 D265A).

Given the limited clinical success with any single agent therapy for overt AML, the therapeutic impact of combining IgG2a anti-U5 snRNP200 antibody with a commonly used therapeutic for AML patients—the nucleoside analog azacytidine was also investigated. Azacitidine is currently being combined with other antibody-based therapeutic approaches in AML including anti-CD47 and anti-TIM-3 antibodies, which provided further motivation for testing in combination with U5 snRNP antibodies. Importantly, combined azacitidine and anti-U5 snRNP200 antibody treatment provided greater survival benefit to recipient mice engrafted with inversion 3 cells than either agent alone or control (FIG. 3H). To interrogate the possible mechanism underlying the superior outcome of the combination therapy group, Fc receptor expression on immune cells in response to in vivo azacitidine therapy was profiled. Interestingly, azacitidine treatment was associated with increased expression of the activating Fc receptor CD16.2 (FcRIV) but also decreased expression of the inhibitory Fc receptor CD32B (FcRIIB) on monocytes and macrophages (CD45.2⁻NK1.1⁻CD11b⁺Ly6c⁺) that resulted in a statistically significant improvement in the ratio of activating to inhibitory receptor expression (FIGS. 3I-3J). These results support U5 snRNP200 targeting as a promising novel therapeutic for AML, identify that anti-U5 snRNP200 antibodies exert maximum therapeutic benefit via activation of FcRs, and highlight a contribution of azacitidine treatment to improving the balance of activating: inhibitory Fc receptor distribution in the AML microenvironment.

### Example 5: Development of Anti-U5 snRNP200 CAR T Cells

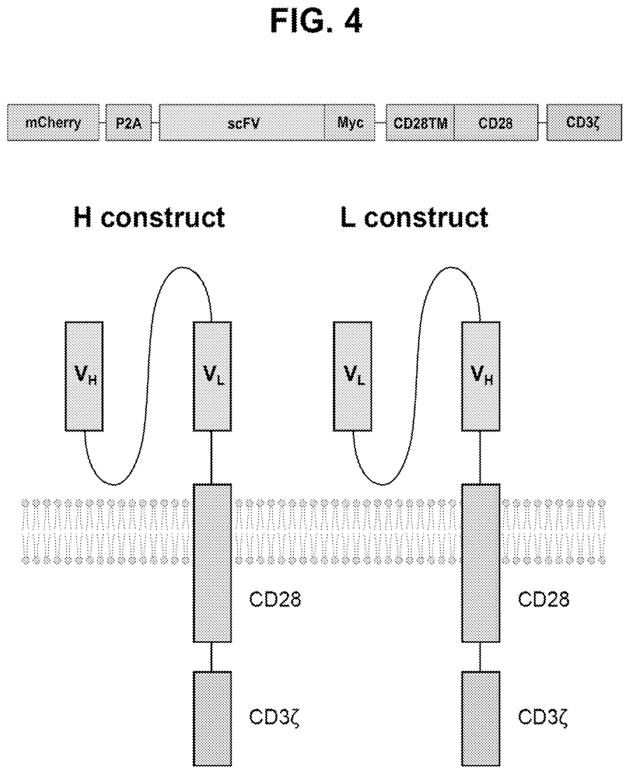

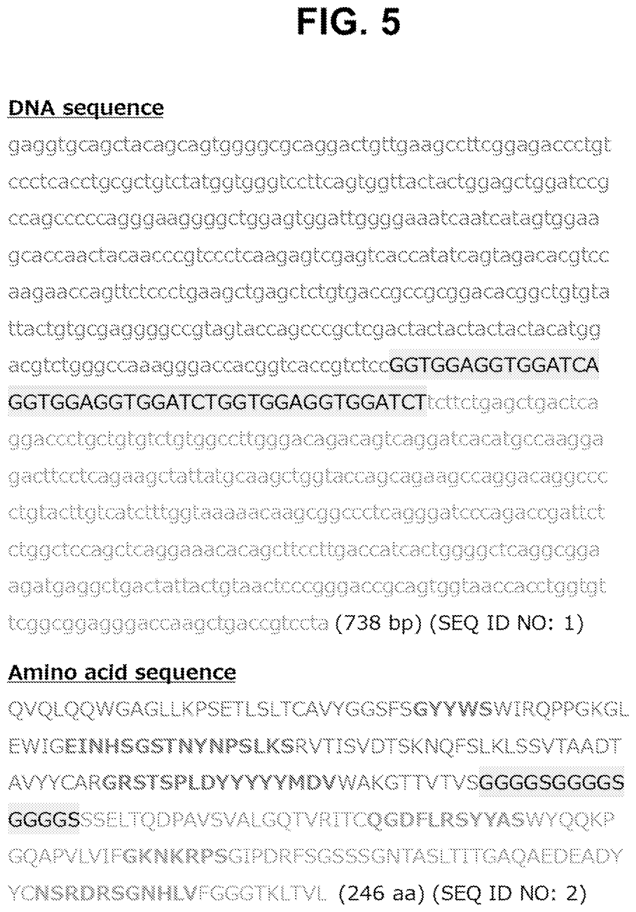

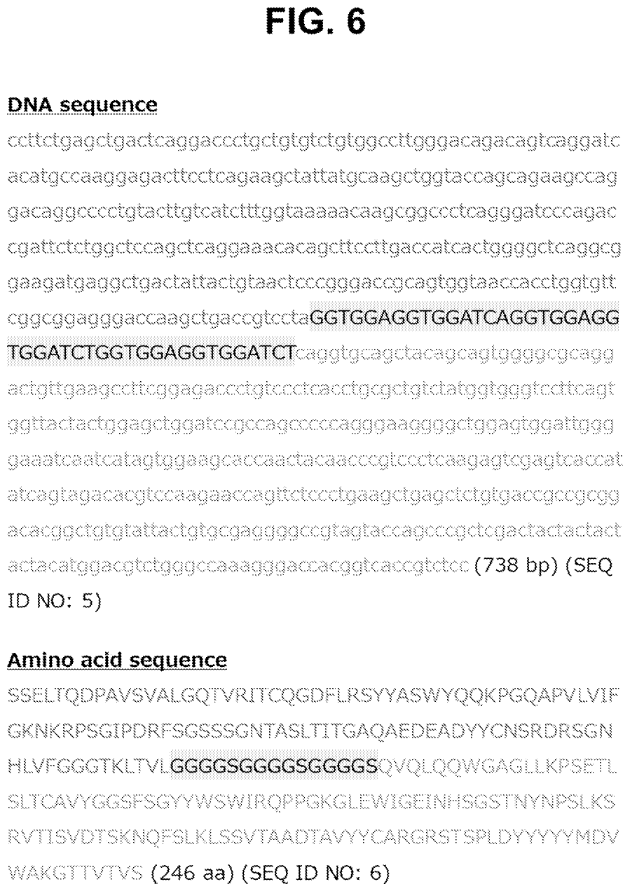

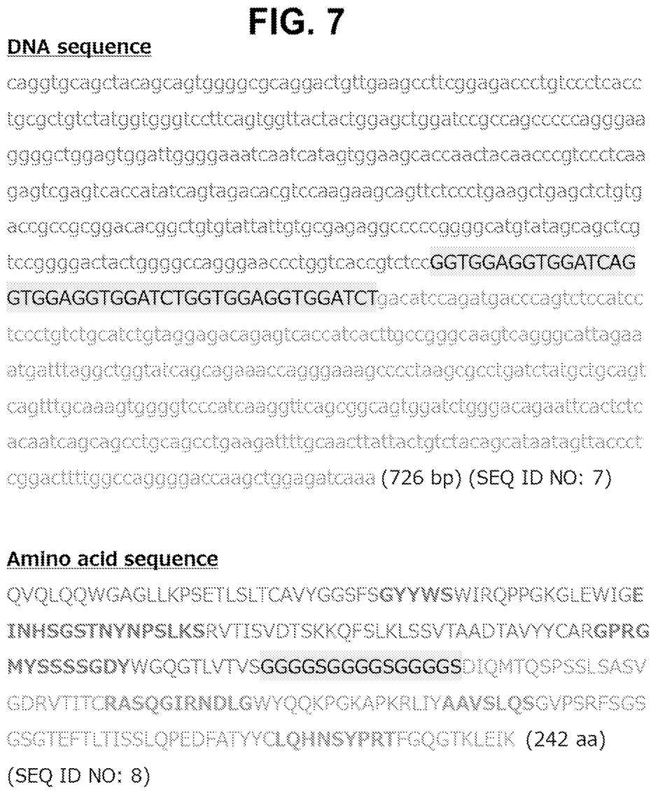

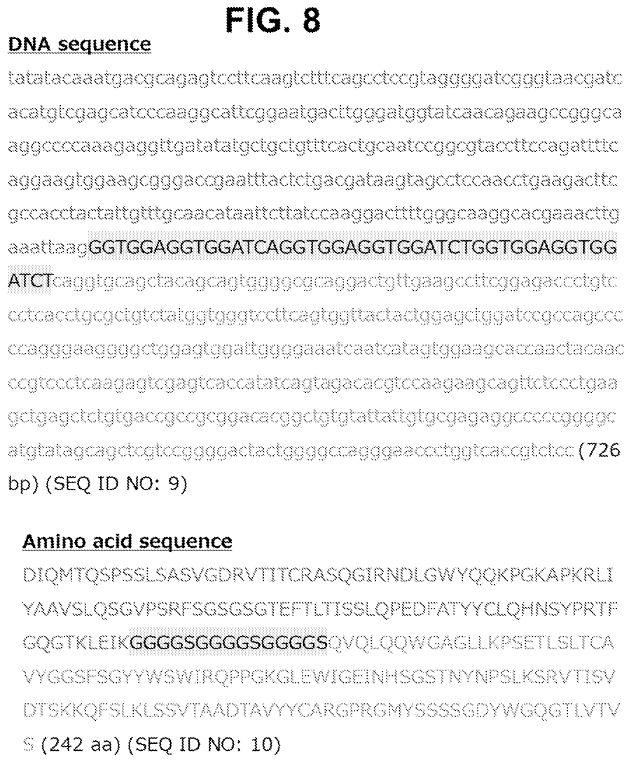

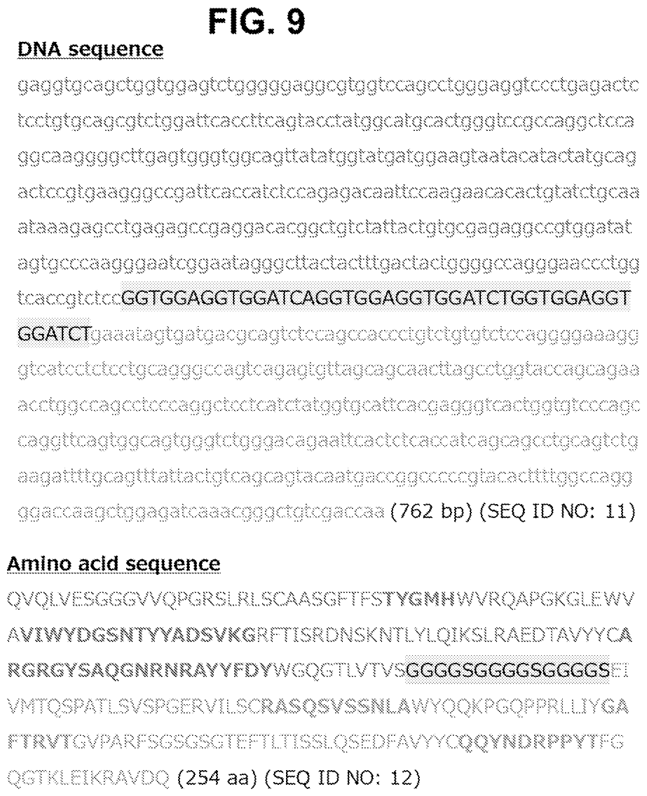

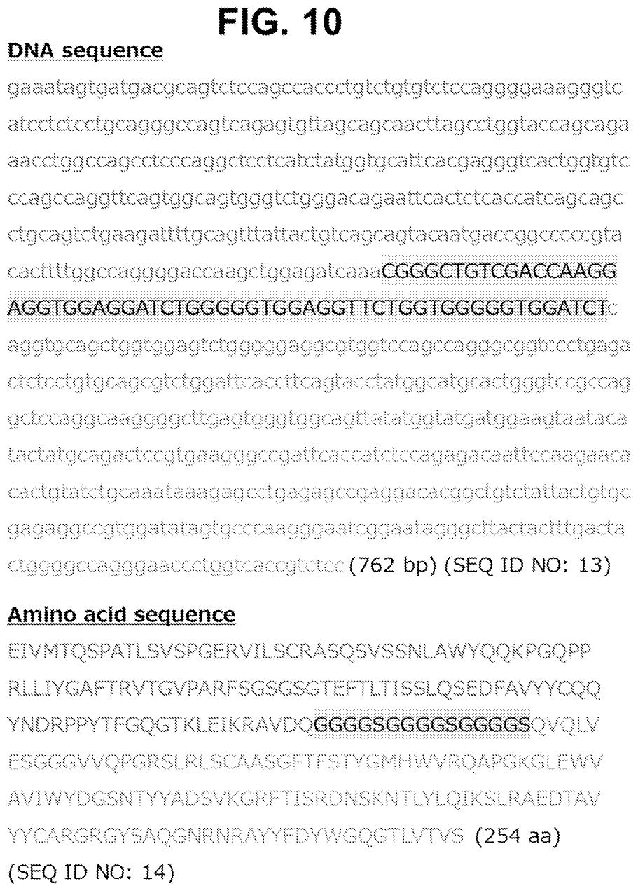

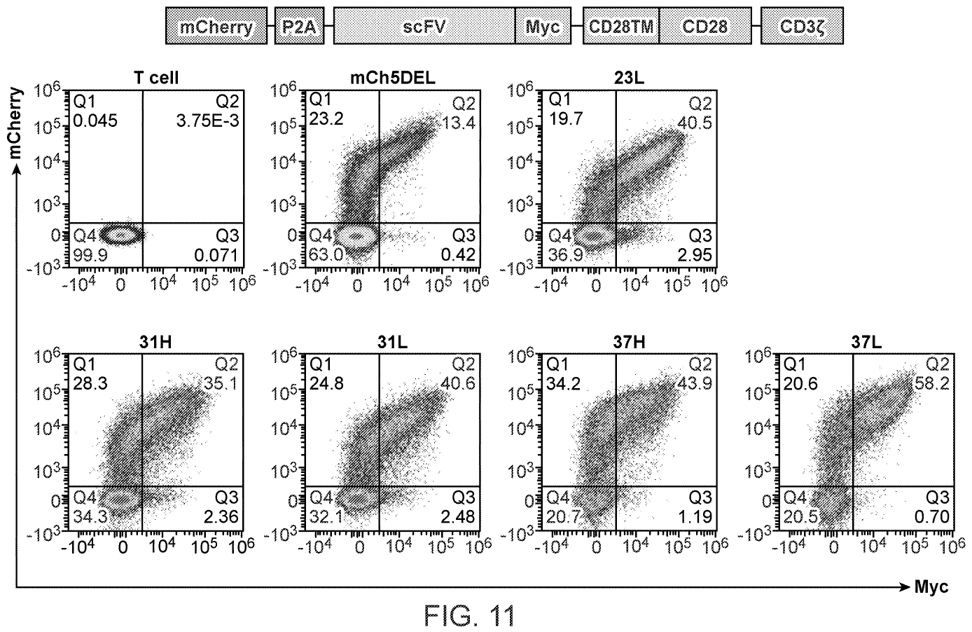

Three U5 snRNP200-directed antibodies [clones 23, 31, and 37] were converted into six single-chain variable fragments (scFvs) with either heavy-chain/light-chain (H), or light-chain/heavy-chain (L) orientation (FIGS. 4-10). These scFvs (23H, 23L, 31H, 31L, 37H and 37L) were successfully integrated into a CD28-based second-generation CAR T cell platform (FIG. 11).

### Example 6: Cytotoxic Activity and Anti-Tumor Effects of Anti-U5 snRNP200 CAR T Cells Against AML Cell Lines

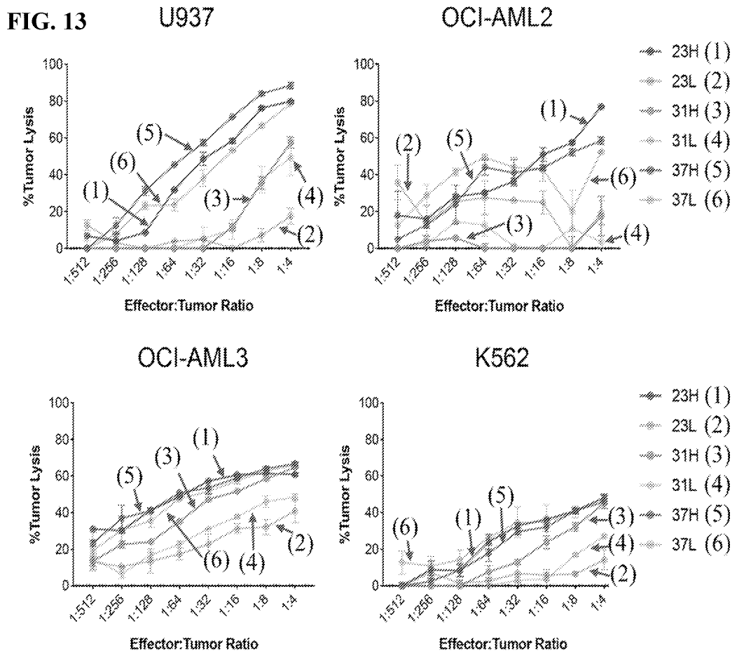

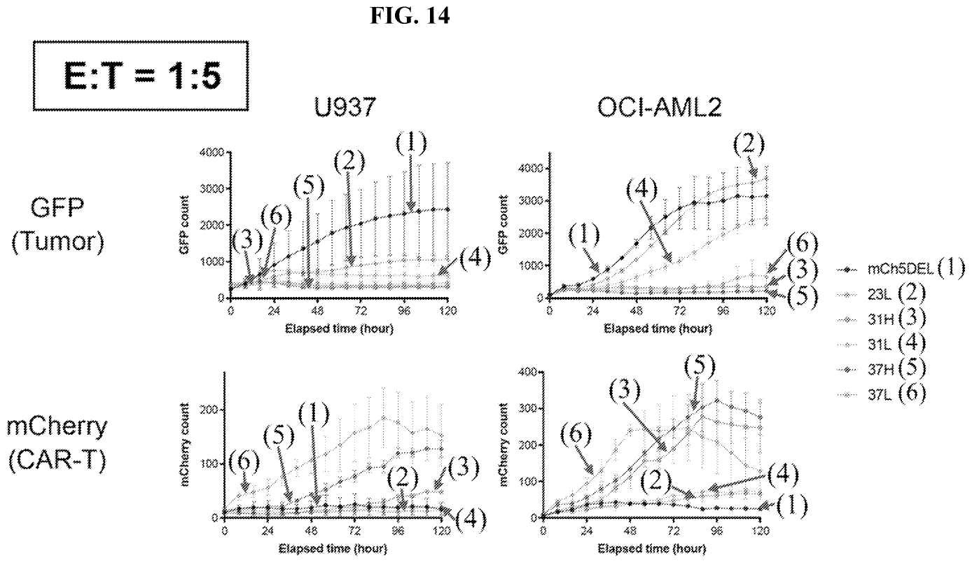

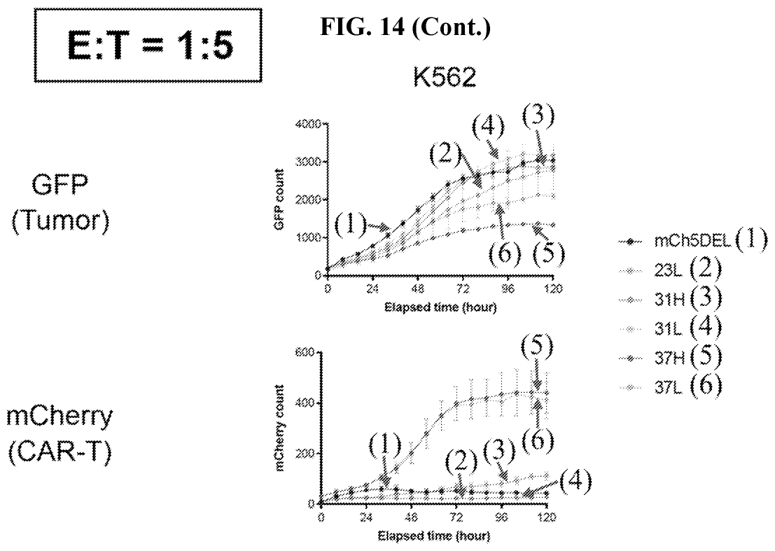

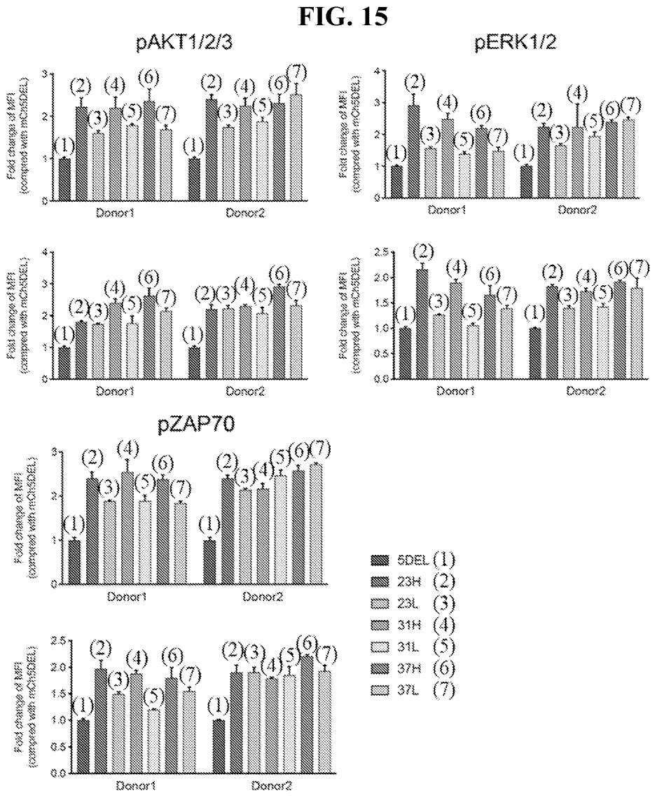

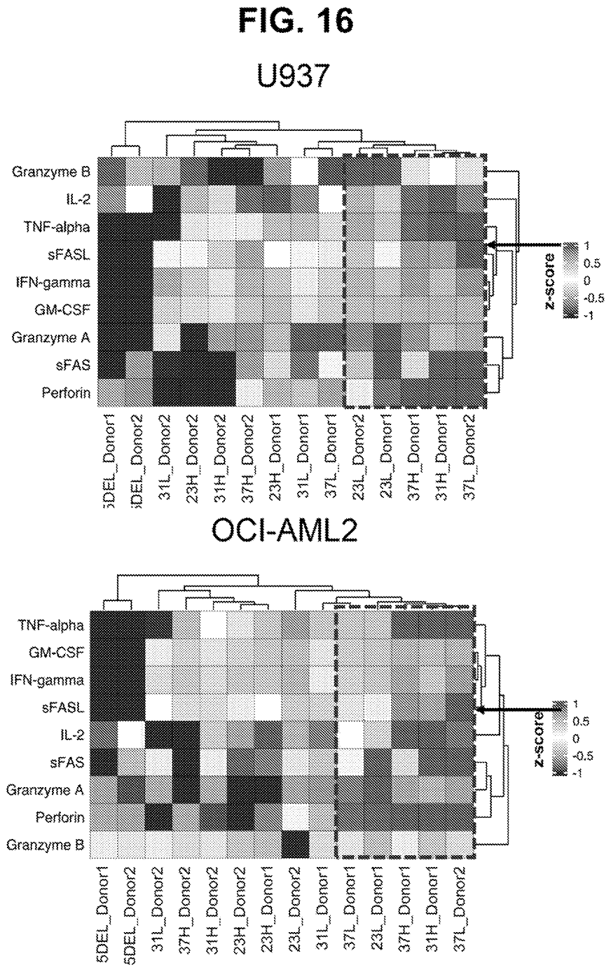

Consistent with the previous findings, the surface expression of U5 snRNP200 was confirmed in a series of AML cell lines (FIG. 12). U5 snRNP200 CAR T cells demonstrated a clear cytotoxic activity and a capacity to proliferate against these cell lines in vitro (FIGS. 13-14) accompanied by activation of T cell receptor signaling and secretion of multiple effector molecules (FIGS. 15-16).

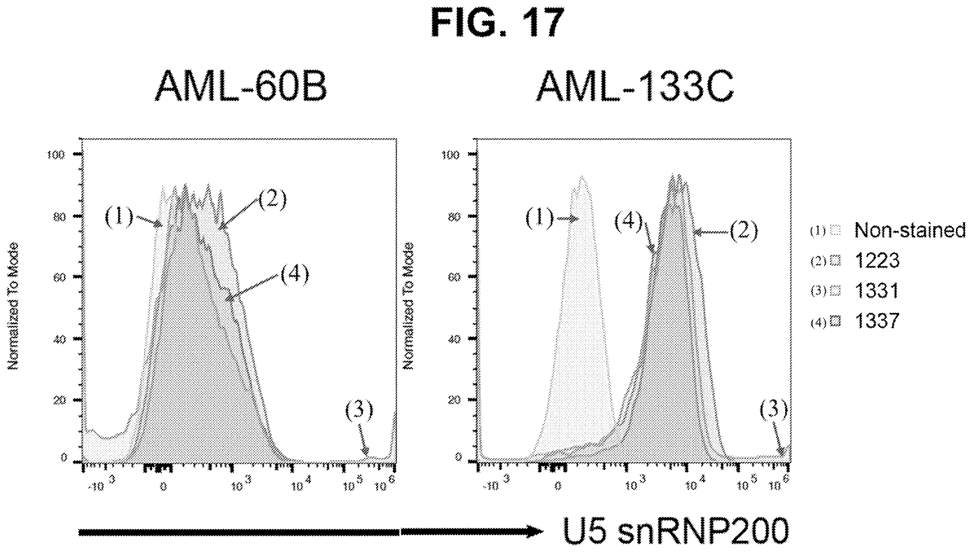

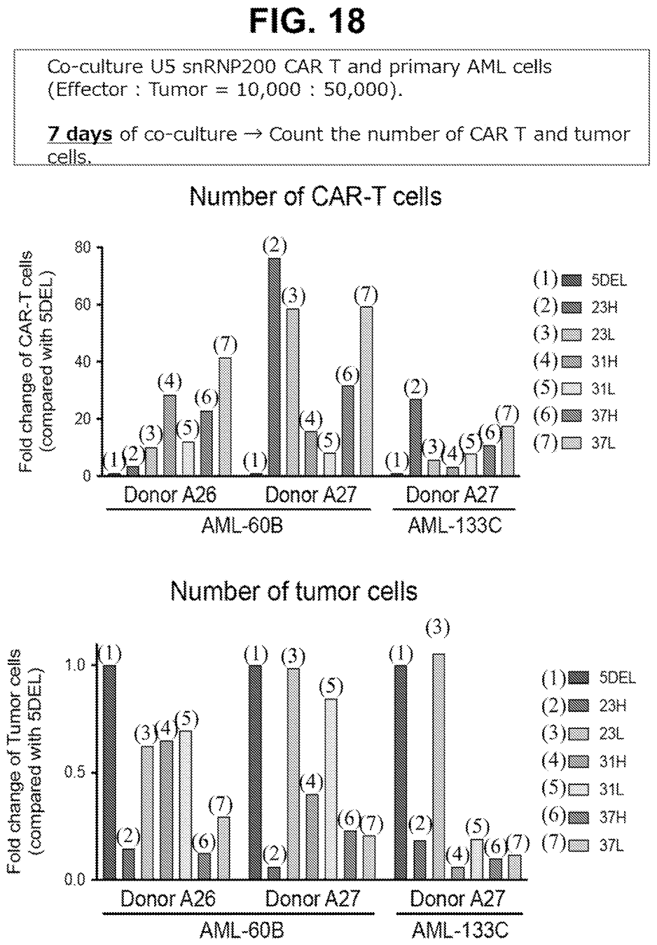

U5 snRNP200 was expressed on the cell surface of primary AML cells derived from two patients (FIG. 17). U5 snRNP200 CAR T cells were proliferative and exhibited anti-tumor effect on these primary AML cells in vitro (FIG. 18).

To evaluate the anti-tumor activity of U5 snRNP200 CAR T cells in vivo, U937 human AML cells and U5 snRNP200 CAR T cells were transplanted into immunodeficient NOD-Prkdc^{em26Cd52}Il2rg^{em26Cd22}/NjuCrl (NCG) mice. U5 snRNP200 CAR T cells expressing 37H and 37L constructs suppressed growth of U937 cells, whereas those expressing 23H did not (FIG. 21), despite the fact that U5 snRNP200 clone 23 antibodies showed higher signal intensity compared with clone 37 antibodies in AML cell lines (FIG. 12).

These results demonstrate that the U5 snRNP200-specific engineered immune cells of the present technology are useful for treating AML.

### Example 7: Efficacy of Anti-U5 snRNP200 CAR T Cells Against B Cell Malignancies

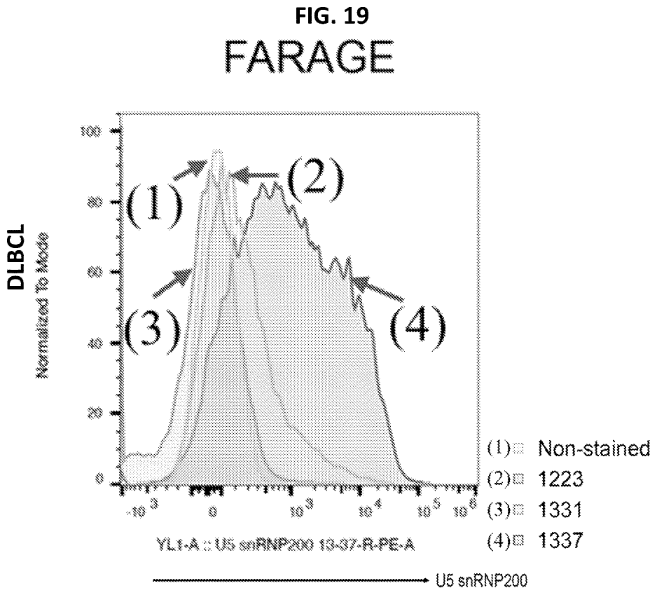

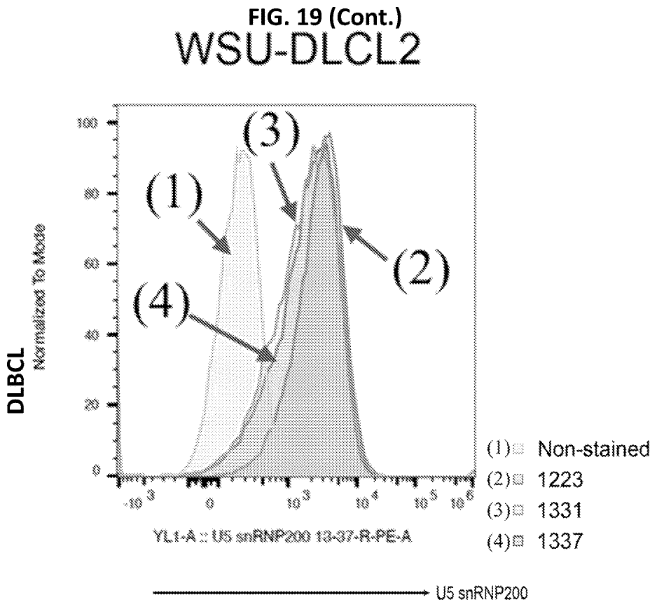

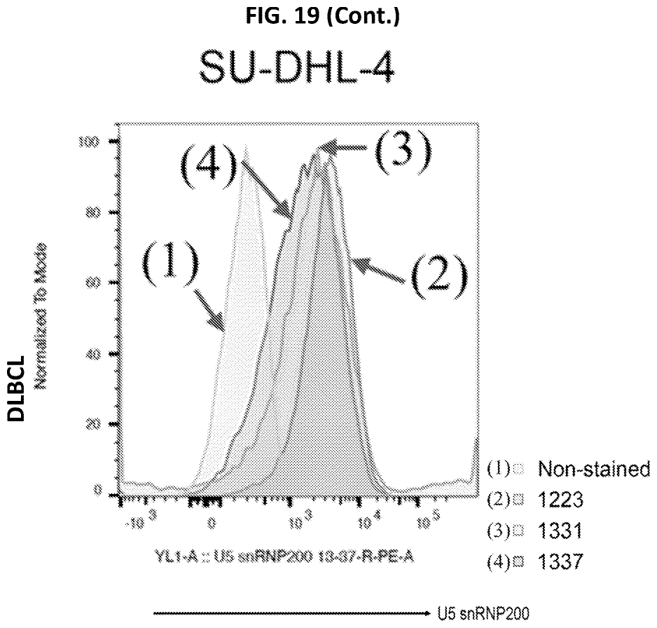

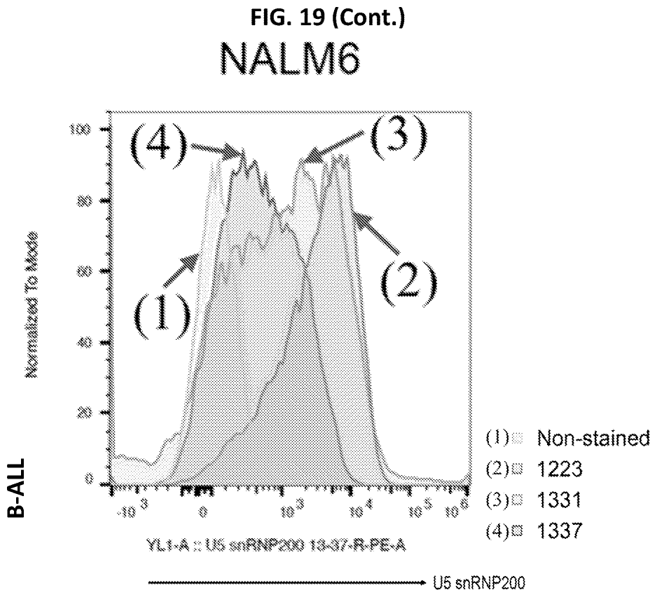

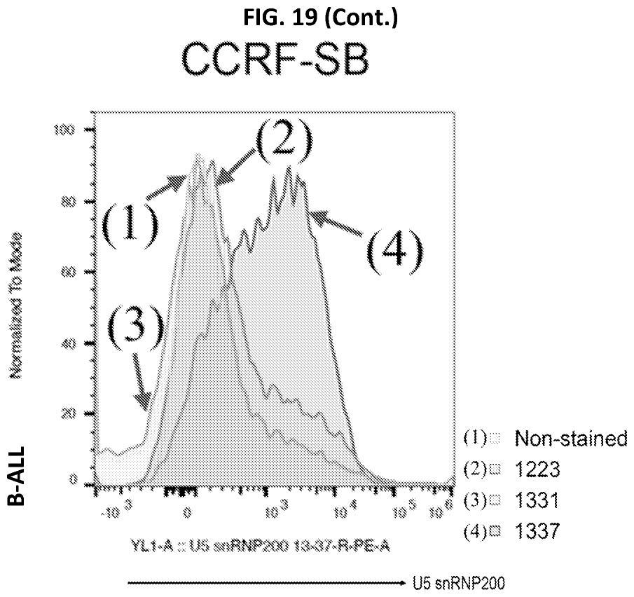

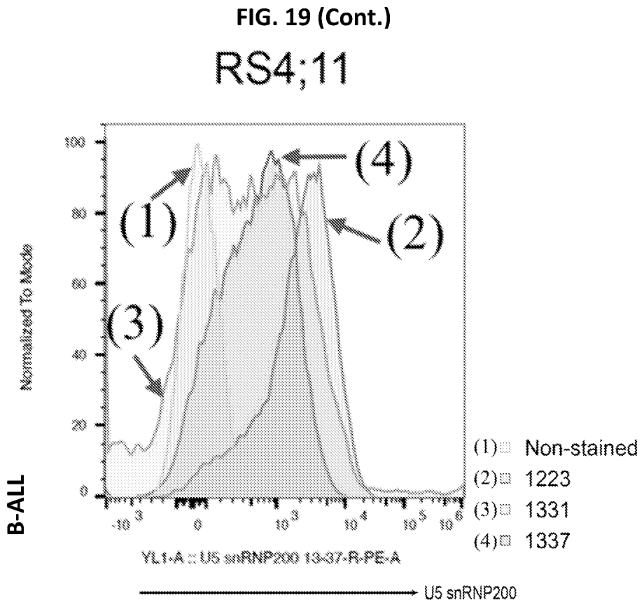

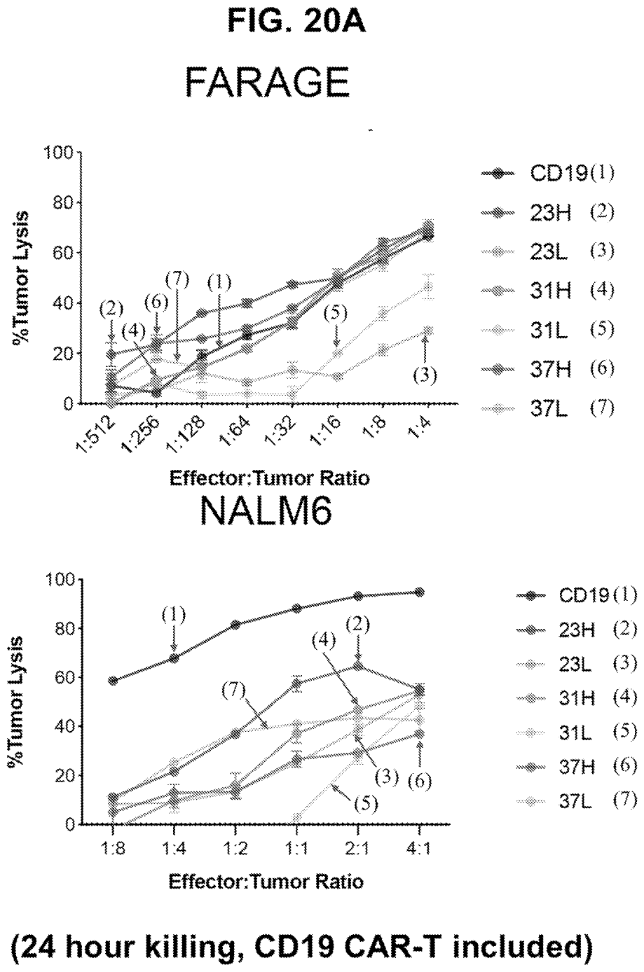

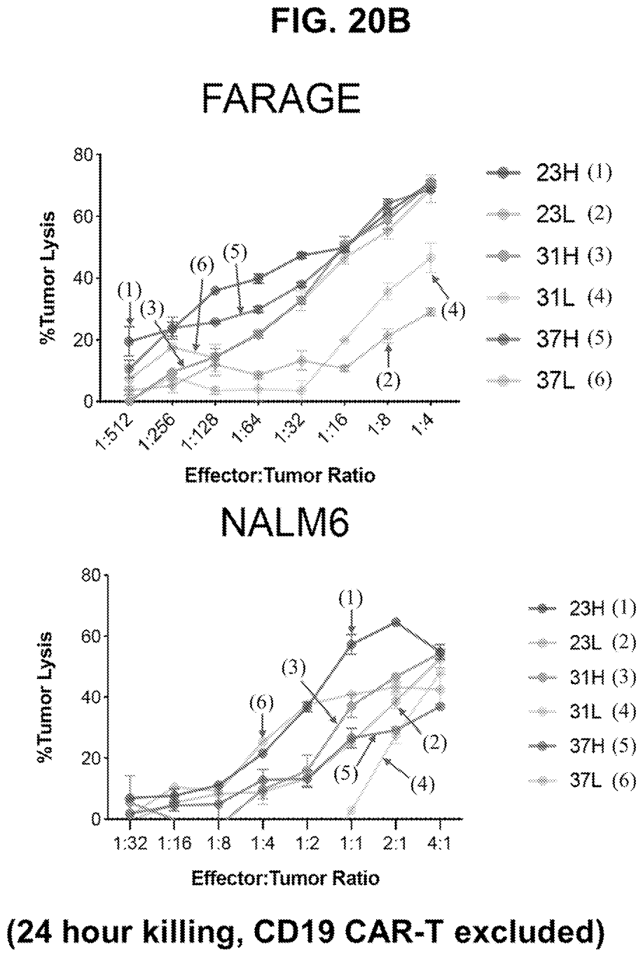

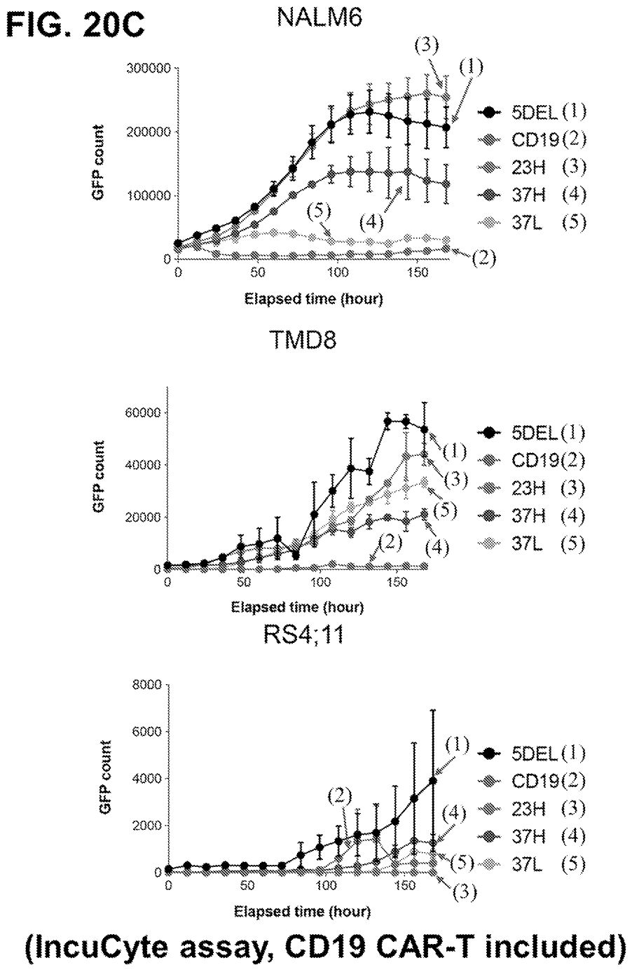

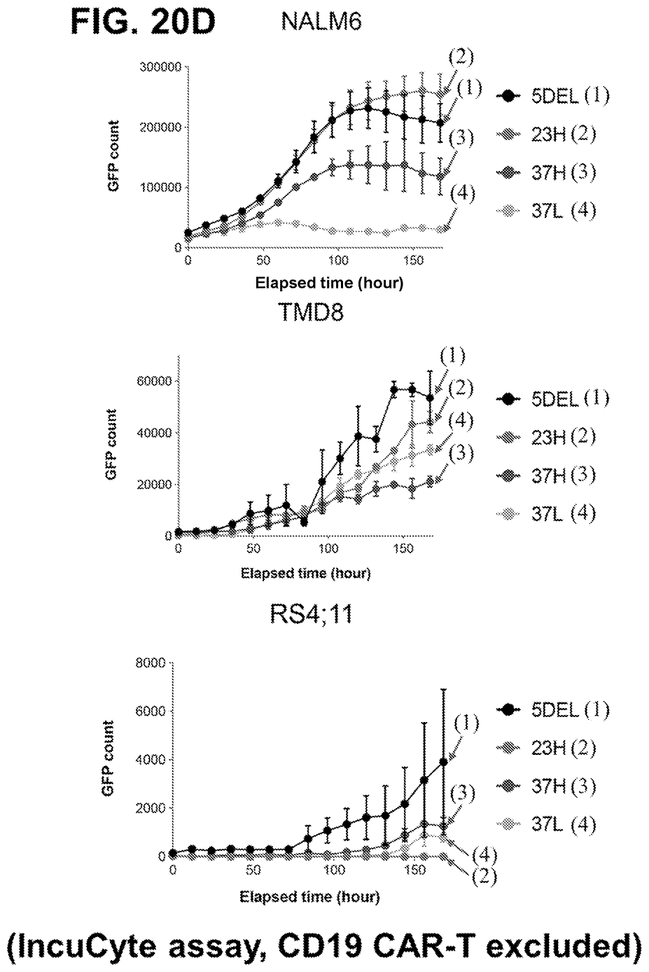

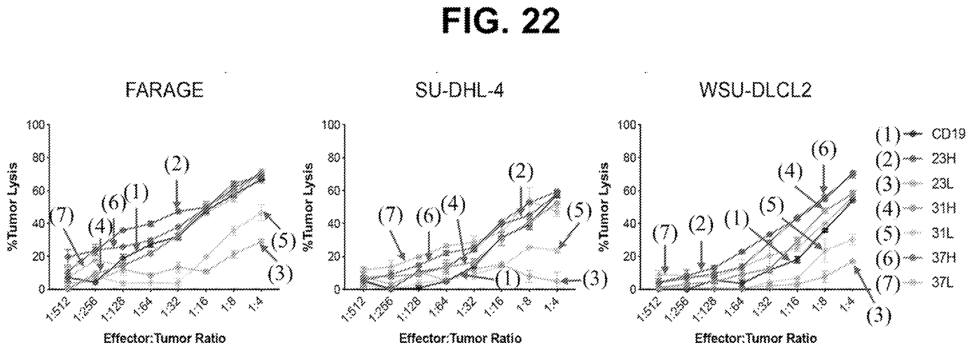

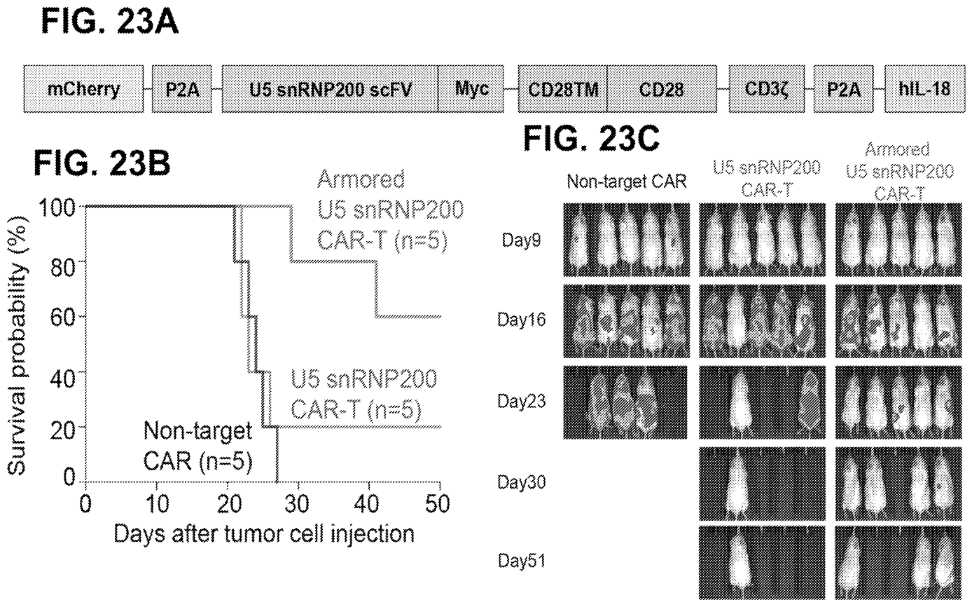

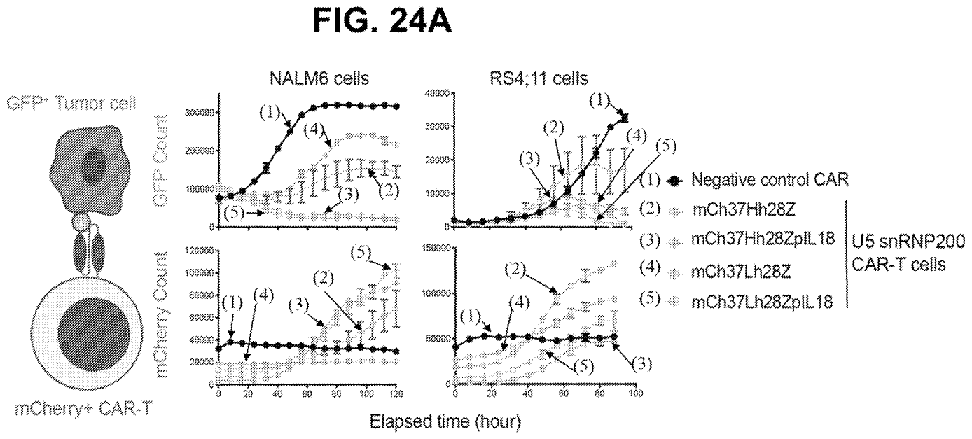

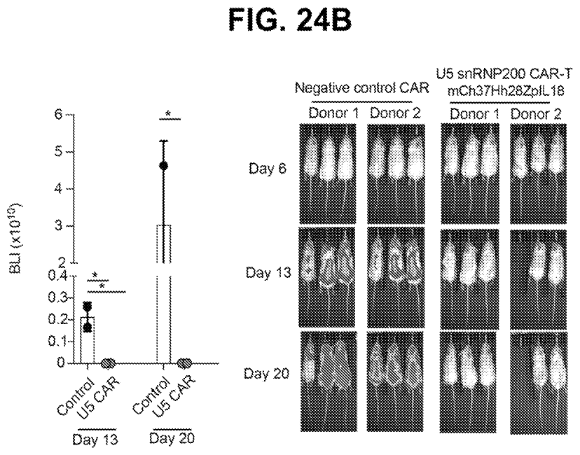

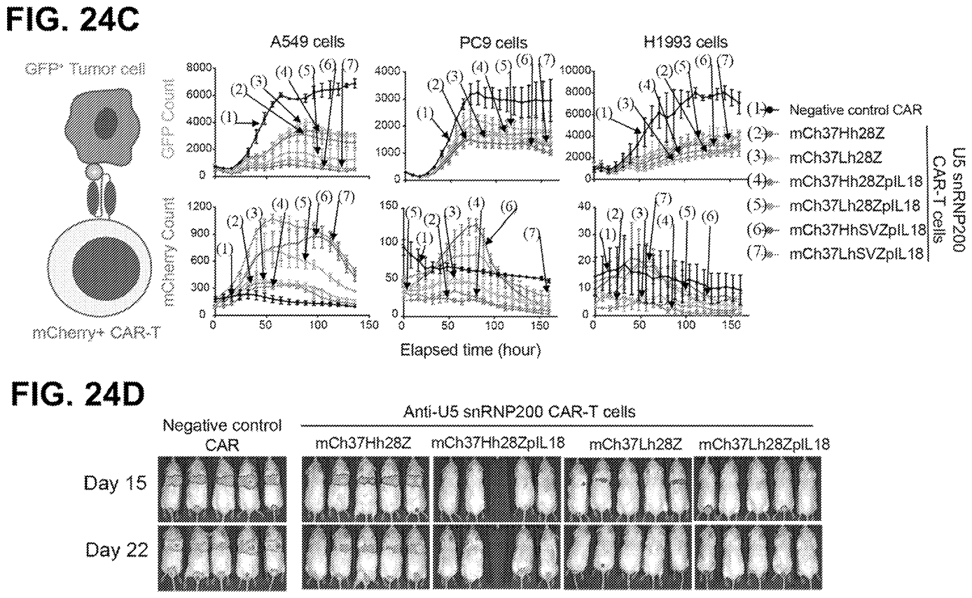

Given the expression of U5 snRNP200 on the surface of normal B-cells, the expression of this antigen on the surface of diffuse large B cell lymphoma (DLBCL) and B-cell acute lymphoblastic leukemia (B-ALL) cell lines was evaluated. These cell lines expressed U5 snRNP200 and were susceptible to U5 snRNP200 CAR T cells (FIGS. 19-20, 22).

These results demonstrate that the U5 snRNP200-specific engineered immune cells of the present technology are useful for treating B cell malignancies.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

### REFERENCES

1. Buccisano F, Rossi F M, Venditti A, et al. CD90/Thy-1 is preferentially expressed on blast cells of high risk acute myeloid leukaemias. Br J Haematol. 2004; 125 (2): 203-212.
2. Arnone M, Konantz M, Hanns P, et al. Acute Myeloid Leukemia Stem Cells: The Challenges of Phenotypic Heterogeneity. Cancers (Basel). 2020; 12 (12).
3. Gillissen M A, Kedde M, Jong G, et al. AML-specific cytotoxic antibodies in patients with durable graft-versus-leukemia responses. Blood. 2018; 131 (1): 131-143.
4. Agafonov D E, Kastner B, Dybkov O, et al. Molecular architecture of the human U4/U6.U5 tri-snRNP. Science. 2016; 351 (6280): 1416-1420.
5. Nimmerjahn F, Ravetch J V. Divergent immunoglobulin g subclass activity through selective Fc receptor binding. Science. 2005; 310 (5753): 1510-1512.
6. Thulin N K, Brewer R C, Sherwood R, et al. Maternal Anti-Dengue IgG Fucosylation Predicts Susceptibility to Dengue Disease in Infants. Cell Rep. 2020; 31 (6): 107642.
7. Zuber J, Shi J, Wang E, et al. RNAi screen identifies Brd4 as a therapeutic target in acute myeloid leukaemia. Nature. 2011; 478 (7370): 524-528.
8. Yamazaki H, Suzuki M, Otsuki A, et al. A remote GATA2 hematopoietic enhancer drives leukemogenesis in inv (3)(q21; q26) by activating EVIL expression. Cancer Cell. 2014; 25 (4): 415-427.
9. Tanaka A, Nakano T A, Nomura M, et al. Aberrant EVI1 splicing contributes to EVI1-rearranged leukemia. Blood. 2022; 140 (8): 875-888.
10. Park L M, Lannigan J, Jaimes M C. OMIP-069: Forty-Color Full Spectrum Flow Cytometry Panel for Deep Immunophenotyping of Major Cell Subsets in Human Peripheral Blood. Cytometry A. 2020; 97 (10): 1044-1051.

1. An engineered immune cell including a receptor that comprises a U5 snRNP200 antigen binding fragment comprising:
a V_{H}CDR1 sequence, a V_{H}CDR2 sequence, and a V_{H}CDR3 sequence of
GYYWS (SEQ ID NO: 17), EINHSGSTNYNPSLKS (SEQ ID NO: 18), and GRSTSPLDYYYYYMDV (SEQ ID NO: 19); or
GYYWS (SEQ ID NO: 23), EINHSGSTNYNPSLKS (SEQ ID NO: 24), and GPRGMYSSSSGDY (SEQ ID NO: 25); or
TYGMH (SEQ ID NO: 29), VIWYDGSNTYYADSVKG (SEQ ID NO: 30), and ARGRGYSAQGNRNRAYYFDY (SEQ ID NO: 31) respectively; and/or
a V_{L}CDR1 sequence, a V_{L}CDR2 sequence, and a V_{L}CDR3 sequence of:
QGDFLRSYYAS (SEQ ID NO: 20), GKNKRPS (SEQ ID NO: 21), and NSRDRSGNHLV (SEQ ID NO: 22); or
RASQGIRNDLG (SEQ ID NO: 26), AAVSLQS (SEQ ID NO: 27), and LOHNSYPRT (SEQ ID NO: 28); or
RASQSVSSNLA (SEQ ID NO: 32), GAFTRVT (SEQ ID NO: 33), and QQYNDRPPYT (SEQ ID NO: 34), respectively, and/or
a nucleic acid encoding the receptor, wherein the receptor is a T cell receptor or a chimeric antigen receptor.

2. The engineered immune cell of claim 1, wherein the U5 snRNP200 antigen binding fragment comprises
a V_{H}CDR1 sequence, a V_{H}CDR2 sequence, and a V_{H}CDR3 sequence of GYYWS (SEQ ID NO: 17), EINHSGSTNYNPSLKS (SEQ ID NO: 18), and GRSTSPLDYYYYYMDV (SEQ ID NO: 19), respectively; and a V_{L}CDR1 sequence, a V_{L}CDR2 sequence, and a V_{L}CDR3 sequence of: QGDFLRSYYAS (SEQ ID NO: 20), GKNKRPS (SEQ ID NO: 21), and NSRDRSGNHLV (SEQ ID NO: 22), respectively; or
a V_{H}CDR1 sequence, a V_{H}CDR2 sequence, and a V_{H}CDR3 sequence of GYYWS (SEQ ID NO: 23), EINHSGSTNYNPSLKS (SEQ ID NO: 24), and GPRGMYSSSSGDY (SEQ ID NO: 25), respectively; and a V_{L}CDR1 sequence, a V_{L}CDR2 sequence, and a V_{L}CDR3 sequence of: RASQGIRNDLG (SEQ ID NO: 26), AAVSLQS (SEQ ID NO: 27), and LQHNSYPRT (SEQ ID NO: 28), respectively; or
a V_{H}CDR1 sequence, a V_{H}CDR2 sequence, and a V_{H}CDR3 sequence of TYGMH (SEQ ID NO: 29), VIWYDGSNTYYADSVKG (SEQ ID NO: 30), and ARGRGYSAQGNRNRAYYFDY (SEQ ID NO: 31) respectively; and a V_{L}CDR1 sequence, a V_{L}CDR2 sequence, and a V_{L}CDR3 sequence of: RASQSVSSNLA (SEQ ID NO: 32), GAFTRVT (SEQ ID NO: 33), and QQYNDRPPYT (SEQ ID NO: 34) respectively; or
wherein the U5 snRNP200 antigen binding fragment comprises a V_{H} amino acid sequence of any one of SEQ ID NOs: 35-37 and/or a V_{L} amino acid sequence of SEQ ID NOs: 38-40, optionally wherein the U5 snRNP200 antigen binding fragment comprises the V_{H} amino acid sequence and the V_{L} amino acid sequence of SEQ ID NO: 35 and SEQ ID NO: 38; SEQ ID NO: 36 and SEQ ID NO: 39; and SEQ ID NO: 37 and SEQ ID NO: 40, respectively.

3. (canceled)

4. (canceled)

5. An engineered immune cell including a receptor that comprises a U5 snRNP200 antigen binding fragment comprising an amino acid sequence selected from the group consisting of: SEQ ID NO: 2, SEQ ID NO: 6, SEQ ID NO: 8, SEQ ID NO: 10, SEQ ID NO: 12, and SEQ ID NO: 14; and/or a nucleic acid encoding the receptor.

6. The engineered immune cell of claim 1, wherein the receptor is a non-native cell receptor, a T cell receptor, a chimeric antigen receptor; or
wherein the nucleic acid encoding the receptor is operably linked to a promoter, optionally wherein the promoter is a constitutive promoter or a conditional promoter, optionally wherein the conditional promoter is induced by binding of the receptor to a U5 snRNP200 antigen; or
wherein the U5 snRNP200 antigen binding fragment is an scFv, a Fab, or a F(ab)2; or
wherein the receptor is linked to a reporter or a selection marker or an IL-18 polypeptide, optionally wherein the reporter or selection marker is GFP or LNGFR or wherein the receptor is linked to the reporter or selection marker or the IL-18 polypeptide via a self-cleaving linker; or
wherein the engineered immune cell is a lymphocyte, optionally wherein the lymphocyte is a T cell, a B cell, or a natural killer (NK) cell, optionally wherein the T cell is a CD4+ T cell or a CD8+ T cell; or
wherein the engineered immune cell is a tumor infiltrating lymphocyte; or
wherein the engineered immune cell is derived from an autologous donor or an allogenic donor.

7. (canceled)

8. (canceled)

9. (canceled)

10. (canceled)

11. (canceled)

12. (canceled)

13. (canceled)

14. (canceled)

15. (canceled)

16. (canceled)

17. The engineered immune cell of claim 6, wherein the chimeric antigen receptor comprises (i) an extracellular antigen binding domain; (ii) a transmembrane domain; and (iii) an intracellular domain; optionally
wherein the extracellular antigen binding domain comprises a single chain variable fragment (scFv), optionally wherein the scFv is a human scFv; or
wherein the extracellular antigen binding domain comprises an anti-U5 snRNP200 scFv having at least 80%, 85%, 90%, 95%, 96%, 97%, 98%, or 99% sequence identity to any one of SEQ ID NOs: 2, 6, 8, 10, 12, or 14, optionally wherein the extracellular antigen binding domain comprises an anti-U5 snRNP200 scFv has the sequence identity of any one of SEQ ID NOs: 2, 6, 8, 10, 12, or 14; or
wherein the extracellular antigen binding domain comprises a signal peptide that is operably linked to the N-terminus of the extracellular antigen binding domain; or
wherein the transmembrane domain comprises a CD8 transmembrane domain or a CD28 transmembrane domain; or
wherein the intracellular domain comprises one or more costimulatory domains, optionally wherein the one or more costimulatory domains are selected from among a CD28 costimulatory domain, a 4-1BB costimulatory domain, an OX40 costimulatory domain, an ICOS costimulatory domain, a DAP-10 costimulatory domain, a PD-1 costimulatory domain, a CTLA-4 costimulatory domain, a LAG-3 costimulatory domain, a 2B4 costimulatory domain, a BTLA costimulatory domain, a CD32-chain, or any combination thereof.

18. (canceled)

19. (canceled)

20. (canceled)

21. (canceled)

22. (canceled)

23. (canceled)

24. (canceled)

25. (canceled)

26. The engineered immune cell of claim 6, further comprising a signal peptide that is operably linked to the N-terminus of the IL-18 polypeptide, optionally wherein the signal peptide operably linked to the N-terminus of the IL-18 polypeptide comprises the amino acid sequence of SEQ ID NO: 79, optionally wherein the IL-18 polypeptide comprises the amino acid sequence of SEQ ID NO: 81, and/or wherein the engineered immune cell comprises a CD28 polypeptide comprising the amino acid sequence of SEQ ID NO: 74 or SEQ ID NO: 83.

27. (canceled)

28. (canceled)

29. (canceled)

30. (canceled)

31. (canceled)

32. (canceled)

33. A polypeptide comprising a chimeric antigen receptor comprising an amino acid sequence of any one of SEQ ID NOs: 2, 6, 8, 10, 12, 14, or 35-40.

34. The polypeptide of claim 33,
wherein the peptide further comprises a self-cleaving peptide located between the chimeric antigen receptor and any one or more of a reporter, a selection marker or an IL-18 polypeptide, optionally wherein the self-cleaving peptide is a P2A self-cleaving peptide; or
wherein the chimeric antigen receptor further comprises a leader sequence, optionally wherein the leader sequence comprises an amino acid sequence of SEQ ID NO: 41, SEQ ID NO: 43, SEQ ID NO: 45 or SEQ ID NO: 47; or
wherein the chimeric antigen receptor comprises (i) an extracellular antigen binding domain; (ii) a transmembrane domain; and (iii) an intracellular domain, optionally wherein the extracellular antigen binding domain binds to a U5 snRNP200 antigen; or wherein the extracellular antigen binding domain comprises a single chain variable fragment (scFv); or wherein the extracellular antigen binding domain comprises an anti-U5 snRNP200 scFv having at least 80%, 85%, 90%, 95%, 96%, 97%, 98%, or 99% sequence identity to any one of SEQ ID NOs: 2, 6, 8, 10, 12, or 14, optionally wherein the extracellular antigen binding domain comprises an anti-U5 snRNP200 scFv has the sequence identity of any one of SEQ ID NOs: 2, 6, 8, 10, 12, or 14; or wherein the transmembrane domain comprises a CD8 transmembrane domain or a CD28 transmembrane domain; or wherein the intracellular domain comprises one or more costimulatory domains, optionally wherein the one or more costimulatory domains are selected from among a CD28 costimulatory domain, a 4-1BB costimulatory domain, an OX40 costimulatory domain, an ICOS costimulatory domain, a DAP-10 costimulatory domain, a PD-1 costimulatory domain, a CTLA-4 costimulatory domain, a LAG-3 costimulatory domain, a 2B4 costimulatory domain, a BTLA costimulatory domain, a CD3C-chain, or any combination thereof.

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. A nucleic acid encoding the polypeptide of claim 34.

47. The nucleic acid of claim 46, wherein the nucleic acid encoding the polypeptide is operably linked to a promoter optionally wherein the promoter is a constitutive promoter or an inducible promoter, optionally wherein the conditional promoter is inducible by the chimeric antigen receptor binding to a U5 snRNP200 antigen.

48. (canceled)

49. (canceled)

50. (canceled)

51. A vector comprising the nucleic acid of claim 46, optionally wherein the vector is a viral vector, a retroviral vector, or a plasmid.

52. (canceled)

53. (canceled)

54. A host cell comprising the nucleic acid of claim 46.

55. A kit comprising the engineered immune cell of claim 1, and instructions for use.

56. A method for preparing immune cells for cancer therapy comprising
isolating immune cells from a donor subject; and
transducing the immune cells with the nucleic acid of claim 46.

57. A method of treatment comprising
isolating immune cells from a donor subject;
transducing the immune cells with the nucleic acid of claim 46; and
administering the transduced immune cells to a recipient subject.

58. The method of claim 57, wherein the donor subject and the recipient subject are the same or are different; or
wherein the immune cells isolated from the donor subject comprise one or more lymphocytes, optionally wherein the one or more lymphocytes is a T cell, a B cell, or a natural killer (NK) cell and optionally wherein the T cell is a CD4+ T cell or a CD8+T; or
wherein the immune cells isolated from the donor subject comprise tumor infiltrating lymphocytes.

59. (canceled)

60. (canceled)

61. (canceled)

62. (canceled)

63. (canceled)

64. A method for treating cancer in a subject in need thereof comprising administering to the subject an effective amount of the engineered immune cell of claim 1.

65. The method of claim 64,
wherein the method further comprises administering to the subject a tumor specific monoclonal antibody; or
wherein the method comprises administering the engineered cell intravenously, intraperitoneally, subcutaneously, intramuscularly, or intratumorally; or
wherein the method further comprises administering an additional cancer therapy, optionally wherein the additional cancer therapy is selected from among chemotherapy, radiation therapy, immunotherapy, monoclonal antibodies, anti-cancer nucleic acids or proteins, anti-cancer viruses or microorganisms, and any combinations thereof; or
wherein the method further comprises administering a cytokine to the subject, optionally wherein the cytokine is selected from the group consisting of interferon α, interferon β, interferon γ, complement C5a, IL-2, TNFalpha, CD40L, IL12, IL-23, IL15, IL17, CCL1, CCL11, CCL12, CCL13, CCL14-1, CCL14-2, CCL14-3, CCL15-1, CCL15-2, CCL16, CCL17, CCL18, CCL19, CCL19, CCL2, CCL20, CCL21, CCL22, CCL23-1, CCL23-2, CCL24, CCL25-1, CCL25-2, CCL26, CCL27, CCL28, CCL3, CCL3L1, CCL4, CCL4L1, CCL5, CCL6, CCL7, CCL8, CCL9, CCR10, CCR2, CCR5, CCR6, CCR7, CCR8, CCRL1, CCRL2, CX3CL1, CX3CR, CXCL1, CXCL10, CXCL11, CXCL12, CXCL13, CXCL14, CXCL15, CXCL16, CXCL2, CXCL3, CXCL4, CXCL5, CXCL6, CXCL7, CXCL8, CXCL9, CXCL9, CXCR1, CXCR2, CXCR4, CXCR5, CXCR6, CXCR7 and XCL2; or
wherein the method further comprises sequentially, separately, or simultaneously administering to the subject at least one therapeutic agent, optionally wherein the at least one therapeutic agent is selected from the group consisting of cytarabine (cytosine arabinoside or ara-C), anthracycline drugs (such as daunorubicin (daunomycin) or idarubicin), cladribine (2-CdA), fludarabine, mitoxantrone, etoposide (VP-16), 6-thioguanine (6-TG), hydroxyurea, corticosteroid drugs (e.g., prednisone or dexamethasone), methotrexate (MTX), 6-mercaptopurine (6-MP), azacytidine, and decitabine or wherein the at least one therapeutic agent is selected from the group consisting of FLT3 inhibitors (e.g., Midostaurin, Gilteritinib), IDH inhibitors (e.g., Ivosidenib, Olutasidenib, Enasidenib, Gemtuzumab ozogamicin), BCL-2 inhibitors (e.g., Venetoclax), Hedgehog pathway inhibitors (e.g., Glasdegib), Tafasitamab, blinatumomab, loncastuximab tesirine, Epratuzumab, Inotuzumab ozogamicin, and hypomethylating agents, or wherein the at least one therapeutic agent is selected from the group consisting of Cytotoxic T-Lymphocyte Associated Protein 4 (CTLA4) inhibitor, a Programmed Cell Death 1 (PD-1) inhibitor, a Programmed Death Ligand 1 (PD-L1) inhibitor, BiTE/DART (e.g., Flotetuzumab, Vibecotamab), Antibody drug conjugates (e.g., IMGN632), Magrolimab, and MBG453+HMA.

66. A method for treating of inhibiting tumor growth or metastasis in a subject with cancer comprising contacting a tumor cell with an effective amount of the engineered immune cell of claim 1.

67. (canceled)

68. (canceled)

69. (canceled)

70. (canceled)

71. (canceled)

72. The method of claim 64, wherein the cancer or tumor is selected from among acute myeloid leukemia (AML), Diffuse large B-cell lymphoma (DLBCL), B-cell acute lymphoblastic leukemia (B-ALL), Follicular lymphoma, Chronic lymphocytic leukemia (CLL)/small lymphocytic lymphoma (SLL), Mantle cell lymphoma (MCL), Extranodal marginal zone B-cell lymphoma, Nodal marginal zone B-cell lymphoma, Splenic marginal zone B-cell lymphoma, Burkitt lymphoma, Lymphoplasmacytic lymphoma (Waldenstrom macroglobulinemia), Hairy cell leukemia, Primary central nervous system (CNS) lymphoma, lung cancer (e.g., non-small cell lung cancer), and metastases thereof or wherein the cancer is a relapsed or refractory cancer.

73. (canceled)

74. (canceled)

75. (canceled)

76. (canceled)

77. (canceled)


SEQUENCE LISTING (86 sequences)

SEQ ID NO: 1 - DNA, 738 bp, synthetic construct
  source 1..738: mol_type = other DNA
gaggtgcagctacagcagtggggcgcaggactgttgaagccttcggagaccctgtccctcacctgcgctgtctatggtgggtccttcagtggttactactggagctggatccgccagcccccagggaaggggctggagtggattggggaaatcaatcatagtggaagcaccaactacaacccgtccctcaagagtcgagtcaccatatcagtagacacgtccaagaaccagttctccctgaagctgagctctgtgaccgccgcggacacggctgtgtattactgtgcgaggggccgtagtaccagcccgctcgactactactactactacatggacgtctgggccaaagggaccacggtcaccgtctccggtggaggtggatcaggtggaggtggatctggtggaggtggatcttcttctgagctgactcaggaccctgctgtgtctgtggccttgggacagacagtcaggatcacatgccaaggagacttcctcagaagctattatgcaagctggtaccagcagaagccaggacaggcccctgtacttgtcatctttggtaaaaacaagcggccctcagggatcccagaccgattctctggctccagctcaggaaacacagcttccttgaccatcactggggctcaggcggaagatgaggctgactattactgtaactcccgggaccgcagtggtaaccacctggtgttcggcggagggaccaagctgaccgtccta

SEQ ID NO: 2 - protein, 246 aa, synthetic construct
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARGRSTSPLDYYYYYMDVWAKGTTVTVSGGGGSGGGGSGGGGSSSELTQDPAVSVALGQTVRITCQGDFLRSYYASWYQQKPGQAPVLVIFGKNKRPSGIPDRFSGSSSGNTASLTITGAQAEDEADYYCNSRDRSGNHLVFGGGTKLTVL

SEQ ID NO: 3 - protein, 15 aa, synthetic construct
GGGGSGGGGSGGGGS

SEQ ID NO: 4 - DNA, 45 bp, synthetic construct
  source 1..45: mol_type = other DNA
ggcggcggcggatctggaggtggtggctcaggtggcggaggctcc

SEQ ID NO: 5 - DNA, 738 bp, synthetic construct
  source 1..738: mol_type = other DNA
ccttctgagctgactcaggaccctgctgtgtctgtggccttgggacagacagtcaggatcacatgccaaggagacttcctcagaagctattatgcaagctggtaccagcagaagccaggacaggcccctgtacttgtcatctttggtaaaaacaagcggccctcagggatcccagaccgattctctggctccagctcaggaaacacagcttccttgaccatcactggggctcaggcggaagatgaggctgactattactgtaactcccgggaccgcagtggtaaccacctggtgttcggcggagggaccaagctgaccgtcctaggtggaggtggatcaggtggaggtggatctggtggaggtggatctcaggtgcagctacagcagtggggcgcaggactgttgaagccttcggagaccctgtccctcacctgcgctgtctatggtgggtccttcagtggttactactggagctggatccgccagcccccagggaaggggctggagtggattggggaaatcaatcatagtggaagcaccaactacaacccgtccctcaagagtcgagtcaccatatcagtagacacgtccaagaaccagttctccctgaagctgagctctgtgaccgccgcggacacggctgtgtattactgtgcgaggggccgtagtaccagcccgctcgactactactactactacatggacgtctgggccaaagggaccacggtcaccgtctcc

SEQ ID NO: 6 - protein, 246 aa, synthetic construct
SSELTQDPAVSVALGQTVRITCQGDFLRSYYASWYQQKPGQAPVLVIFGKNKRPSGIPDRFSGSSSGNTASLTITGAQAEDEADYYCNSRDRSGNHLVFGGGTKLTVLGGGGSGGGGSGGGGSQVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARGRSTSPLDYYYYYMDVWAKGTTVTVS

SEQ ID NO: 7 - DNA, 726 bp, synthetic construct
  source 1..726: mol_type = other DNA
caggtgcagctacagcagtggggcgcaggactgttgaagccttcggagaccctgtccctcacctgcgctgtctatggtgggtccttcagtggttactactggagctggatccgccagcccccagggaaggggctggagtggattggggaaatcaatcatagtggaagcaccaactacaacccgtccctcaagagtcgagtcaccatatcagtagacacgtccaagaagcagttctccctgaagctgagctctgtgaccgccgcggacacggctgtgtattattgtgcgagaggcccccggggcatgtatagcagctcgtccggggactactggggccagggaaccctggtcaccgtctccggtggaggtggatcaggtggaggtggatctggtggaggtggatctgacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcagtcagtttgcaaagtggggtcccatcaaggttcagcggcagtggatctgggacagaattcactctcacaatcagcagcctgcagcctgaagattttgcaacttattactgtctacagcataatagttaccctcggacttttggccaggggaccaagctggagatcaaa

SEQ ID NO: 8 - protein, 242 aa, synthetic construct
QVQLQQWGAGLLKPSETLSLTCAVYGGSFSGYYWSWIRQPPGKGLEWIGEINHSGSTNYNPSLKSRVTISVDTSKKQFSLKLSSVTAADTAVYYCARGPRGMYSSSSGDYWGQGTLVTVSGGGGSGGGGSGGGGSDIQMTQSPSSLSASVGDRVTITCRASQGIRNDLGWYQQKPGKAPKRLIYAAVSLQSGVPSRFSGSGSGTEFTLTISSLQPEDFATYYCLQHNSYPRTFGQGTKLEIK

SEQ ID NO: 9 - DNA, 726 bp, synthetic construct
  source 1..726: mol_type = other DNA
tatatacaaatgacgcagagtccttcaagtctttcagcctccgtaggggatcgggtaacgatcacatgtcgagcatcccaaggcattcggaatgacttgggatggtatcaacagaagccgggcaaggccccaaagaggttgatatatgctgctgtttcactgcaatccggcgtaccttccagattttcaggaagtggaagcgggaccgaatttactctgacgataagtagcctccaacctgaagacttcgccacctactattgtttgcaacataattcttatccaaggacttttgggcaaggcacgaaacttgaaattaagggtggaggtggatcaggtggaggtggatctggtggaggtggatctcaggtgcagctacagcagtggggcgcaggactgttgaagccttcggagaccctgtccctcacctgcgctgtctatggtgggtccttcagtggttactactggagctggatccgccagcccccagggaaggggctggagtggattggggaaatcaatcatagtggaagcaccaactacaacccgtccctcaagagtcgagtcaccatatcagtagacacgtccaagaagcagttctccctgaagctgagctctgtgaccgccgcggacacggctgtgtattattgtgcgagaggcccccggggcatgtatagcagctcgtccggggactactggggccagggaaccctggtcaccgtctcc

SEQ ID NO: 10 - protein, 242 aa, synthetic construct
DIQMTQSPSSLSASVGDRVTITCRASQGIRNDLGWYQQKPGKAPKRLIYAAVSLQSGVPSRFSGSGSGTEFTLTISSLQPEDFATYYCLQHNSYPRTFGQGTKLEIKGGGGSGGGGSGGGGSQVQLQQWGAGLLKPSETLSLTCAVYGGSFSGYYWSWIRQPPGKGLEWIGEINHSGSTNYNPSLKSRVTISVDTSKKQFSLKLSSVTAADTAVYYCARGPRGMYSSSSGDYWGQGTLVTVS

SEQ ID NO: 11 - DNA, 762 bp, synthetic construct
  source 1..762: mol_type = other DNA
gaggtgcagctggtggagtctgggggaggcgtggtccagcctgggaggtccctgagactctcctgtgcagcgtctggattcaccttcagtacctatggcatgcactgggtccgccaggctccaggcaaggggcttgagtgggtggcagttatatggtatgatggaagtaatacatactatgcagactccgtgaagggccgattcaccatctccagagacaattccaagaacacactgtatctgcaaataaagagcctgagagccgaggacacggctgtctattactgtgcgagaggccgtggatatagtgcccaagggaatcggaatagggcttactactttgactactggggccagggaaccctggtcaccgtctccggtggaggtggatcaggtggaggtggatctggtggaggtggatctgaaatagtgatgacgcagtctccagccaccctgtctgtgtctccaggggaaagggtcatcctctcctgcagggccagtcagagtgttagcagcaacttagcctggtaccagcagaaacctggccagcctcccaggctcctcatctatggtgcattcacgagggtcactggtgtcccagccaggttcagtggcagtgggtctgggacagaattcactctcaccatcagcagcctgcagtctgaagattttgcagtttattactgtcagcagtacaatgaccggcccccgtacacttttggccaggggaccaagctggagatcaaacgggctgtcgaccaa

SEQ ID NO: 12 - protein, 254 aa, synthetic construct
QVQLVESGGGVVQPGRSLRLSCAASGFTFSTYGMHWVRQAPGKGLEWVAVIWYDGSNTYYADSVKGRFTISRDNSKNTLYLQIKSLRAEDTAVYYCARGRGYSAQGNRNRAYYFDYWGQGTLVTVSGGGGSGGGGSGGGGSEIVMTQSPATLSVSPGERVILSCRASQSVSSNLAWYQQKPGQPPRLLIYGAFTRVTGVPARFSGSGSGTEFTLTISSLQSEDFAVYYCQQYNDRPPYTFGQGTKLEIKRAVDQ

SEQ ID NO: 13 - DNA, 762 bp, synthetic construct
  source 1..762: mol_type = other DNA
gaaatagtgatgacgcagtctccagccaccctgtctgtgtctccaggggaaagggtcatcctctcctgcagggccagtcagagtgttagcagcaacttagcctggtaccagcagaaacctggccagcctcccaggctcctcatctatggtgcattcacgagggtcactggtgtcccagccaggttcagtggcagtgggtctgggacagaattcactctcaccatcagcagcctgcagtctgaagattttgcagtttattactgtcagcagtacaatgaccggcccccgtacacttttggccaggggaccaagctggagatcaaacgggctgtcgaccaaggaggtggaggatctgggggtggaggttctggtgggggtggatctcaggtgcagctggtggagtctgggggaggcgtggtccagccagggcggtccctgagactctcctgtgcagcgtctggattcaccttcagtacctatggcatgcactgggtccgccaggctccaggcaaggggcttgagtgggtggcagttatatggtatgatggaagtaatacatactatgcagactccgtgaagggccgattcaccatctccagagacaattccaagaacacactgtatctgcaaataaagagcctgagagccgaggacacggctgtctattactgtgcgagaggccgtggatatagtgcccaagggaatcggaatagggcttactactttgactactggggccagggaaccctggtcaccgtctcc

SEQ ID NO: 14 - protein, 254 aa, synthetic construct
EIVMTQSPATLSVSPGERVILSCRASQSVSSNLAWYQQKPGQPPRLLIYGAFTRVTGVPARFSGSGSGTEFTLTISSLQSEDFAVYYCQQYNDRPPYTFGQGTKLEIKRAVDQGGGGSGGGGSGGGGSQVQLVESGGGVVQPGRSLRLSCAASGFTFSTYGMHWVRQAPGKGLEWVAVIWYDGSNTYYADSVKGRFTISRDNSKNTLYLQIKSLRAEDTAVYYCARGRGYSAQGNRNRAYYFDYWGQGTLVTVS

SEQ ID NO: 15 - protein, 8 aa, synthetic construct
  VARIANT 2: note = V or I
  VARIANT 4: note = Any amino acid
DXEXNPGP

SEQ ID NO: 16 - protein, 75 aa, synthetic construct
  VARIANT 1..75: note = This sequence may encompass 1-15 GGGGS repeating units
GGGGSGGGGSGGGGSGGGGSGGGGSGGGGSGGGGSGGGGSGGGGSGGGGSGGGGSGGGGSGGGGSGGGGSGGGGS

SEQ ID NO: 17 - protein, 5 aa, synthetic construct
GYYWS

SEQ ID NO: 18 - protein, 16 aa, synthetic construct
EINHSGSTNYNPSLKS

SEQ ID NO: 19 - protein, 16 aa, synthetic construct
GRSTSPLDYYYYYMDV

SEQ ID NO: 20 - protein, 11 aa, synthetic construct
QGDFLRSYYAS

SEQ ID NO: 21 - protein, 7 aa, synthetic construct
GKNKRPS

SEQ ID NO: 22 - protein, 11 aa, synthetic construct
NSRDRSGNHLV

SEQ ID NO: 23 - protein, 5 aa, synthetic construct
GYYWS

SEQ ID NO: 24 - protein, 16 aa, synthetic construct
EINHSGSTNYNPSLKS

SEQ ID NO: 25 - protein, 13 aa, synthetic construct
GPRGMYSSSSGDY

SEQ ID NO: 26 - protein, 11 aa, synthetic construct
RASQGIRNDLG

SEQ ID NO: 27 - protein, 7 aa, synthetic construct
AAVSLQS

SEQ ID NO: 28 - protein, 9 aa, synthetic construct
LQHNSYPRT

SEQ ID NO: 29 - protein, 5 aa, synthetic construct
TYGMH

SEQ ID NO: 30 - protein, 17 aa, synthetic construct
VIWYDGSNTYYADSVKG

SEQ ID NO: 31 - protein, 20 aa, synthetic construct
ARGRGYSAQGNRNRAYYFDY

SEQ ID NO: 32 - protein, 11 aa, synthetic construct
RASQSVSSNLA

SEQ ID NO: 33 - protein, 7 aa, synthetic construct
GAFTRVT

SEQ ID NO: 34 - protein, 10 aa, synthetic construct
QQYNDRPPYT

SEQ ID NO: 35 - protein, 123 aa, synthetic construct
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARGRSTSPLDYYYYYMDVWAKGTTVTVS

SEQ ID NO: 36 - protein, 120 aa, synthetic construct
QVQLQQWGAGLLKPSETLSLTCAVYGGSFSGYYWSWIRQPPGKGLEWIGEINHSGSTNYNPSLKSRVTISVDTSKKQFSLKLSSVTAADTAVYYCARGPRGMYSSSSGDYWGQGTLVTVS

SEQ ID NO: 37 - protein, 126 aa, synthetic construct
QVQLVESGGGVVQPGRSLRLSCAASGFTFSTYGMHWVRQAPGKGLEWVAVIWYDGSNTYYADSVKGRFTISRDNSKNTLYLQIKSLRAEDTAVYYCARGRGYSAQGNRNRAYYFDYWGQGTLVTVS

SEQ ID NO: 38 - protein, 108 aa, synthetic construct
SSELTQDPAVSVALGQTVRITCQGDFLRSYYASWYQQKPGQAPVLVIFGKNKRPSGIPDRFSGSSSGNTASLTITGAQAEDEADYYCNSRDRSGNHLVFGGGTKLTVL

SEQ ID NO: 39 - protein, 107 aa, synthetic construct
DIQMTQSPSSLSASVGDRVTITCRASQGIRNDLGWYQQKPGKAPKRLIYAAVSLQSGVPSRFSGSGSGTEFTLTISSLQPEDFATYYCLQHNSYPRTFGQGTKLEIK

SEQ ID NO: 40 - protein, 113 aa, synthetic construct
EIVMTQSPATLSVSPGERVILSCRASQSVSSNLAWYQQKPGQPPRLLIYGAFTRVTGVPARFSGSGSGTEFTLTISSLQSEDFAVYYCQQYNDRPPYTFGQGTKLEIKRAVDQ

SEQ ID NO: 41 - protein, 21 aa, Homo sapiens
MALPVTALLLPLALLLHAARP

SEQ ID NO: 42 - DNA, 63 bp, Homo sapiens
  source 1..63: mol_type = genomic DNA
atggccctgccagtaacggctctgctgctgccacttgctctgctcctccatgcagccaggcct

SEQ ID NO: 43 - protein, 18 aa, Homo sapiens
MALPVTALLLPLALLLHA

SEQ ID NO: 44 - DNA, 54 bp, Homo sapiens
  source 1..54: mol_type = genomic DNA
atggctctcccagtgactgccctactgcttcccctagcgcttctcctgcatgca

SEQ ID NO: 45 - protein, 21 aa, Mus sp.
MASPLTRFLSLNLLLLGESII

SEQ ID NO: 46 - DNA, 63 bp, Mus sp.
  source 1..63: mol_type = genomic DNA
atggccagccccctgaccaggttcctgagcctgaacctgctgctgctgggcgagagcatcatc

SEQ ID NO: 47 - protein, 18 aa, Mus sp.
MASPLTRFLSLNLLLLGE

SEQ ID NO: 48 - DNA, 54 bp, Mus sp.
  source 1..54: mol_type = genomic DNA
atggccagccccctgaccaggttcctgagcctgaacctgctgctgctgggcgag

SEQ ID NO: 49 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 50 - DNA, 321 bp, Homo sapiens
  source 1..321: mol_type = genomic DNA
attgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 51 - protein, 234 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPWKSGDKPSLSARYV

SEQ ID NO: 52 - protein, 71 aa, Homo sapiens
PTTTPAPRPPTPAPTIASQPLSLRPEACRPAAGGAVHTRGLDFACDIYIWAPLAGTCGVLLLSLVITLYCN

SEQ ID NO: 53 - DNA, 213 bp, Homo sapiens
  source 1..213: mol_type = genomic DNA
cccaccacgacgccagcgccgcgaccaccaaccccggcgcccacgatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttactgcaac

SEQ ID NO: 54 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 55 - protein, 164 aa, Homo sapiens; identical to SEQ ID NO: 54

SEQ ID NO: 56 - protein, 112 aa, Homo sapiens
RVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 57 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 58 - DNA, 337 bp, Homo sapiens
  source 1..337: mol_type = genomic DNA
agagtgaagttcagcaggagcgcagagccccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcg

SEQ ID NO: 59 - DNA, 339 bp, Homo sapiens
  source 1..339: mol_type = genomic DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 60 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 61 - protein, 42 aa, Homo sapiens
KRGRKKLLYIFKQPFMRPVQTTQEEDGCSCRFPEEEEGGCEL

SEQ ID NO: 62 - DNA, 126 bp, Homo sapiens
  source 1..126: mol_type = genomic DNA
aaacggggcagaaagaagctcctgtatatattcaaacaaccatttatgagaccagtacaaactactcaagaggaagatggctgtagctgccgatttccagaagaagaagaaggaggatgtgaactg

SEQ ID NO: 63 - protein, 277 aa, Homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 64 - protein, 199 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 65 - protein, 4 aa, synthetic construct
YVKM

SEQ ID NO: 66 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 67 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 68 - protein, 525 aa, Homo sapiens
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 69 - protein, 370 aa, Homo sapiens
MLGQVVTLILLLLLKVYQGKGCQGSADHVVSISGVPLQLQPNSIQTKVDSIAWKKLLPSQNGFHHILKWENGSLPSNTSNDRFSFIVKNLSLLIKAAQQQDSGLYCLEVTSISGKVQTATFQVFVFESLLPDKVEKPRLQGQGKILDRGRCQVALSCLVSRDGNVSYAWYRGSKLIQTAGNLTYLDEEVDINGTHTYTCNVSNPVSWESHTLNLTQDCQNAHQEFRFWPFLVIIVILSALFLGTLACFCVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 70 - protein, 289 aa, Homo sapiens
MKTLPAMLGTGKLFWVFFLIPYLDIWNIHGKESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYRLLPLGGLPLLITTCFCLFCCLRRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 71 - DNA, 46 bp, synthetic construct
  source 1..46: mol_type = other DNA
gtgtggaaaggacgaaacaccgagttttagagctagaaatagcaag

SEQ ID NO: 72 - protein, 10 aa, synthetic construct
EQKLISEEDL

SEQ ID NO: 73 - DNA, 30 bp, synthetic construct
  source 1..30: mol_type = other DNA
gaacagaaactgatctctgaagaagacctg

SEQ ID NO: 74 - protein, 110 aa, Homo sapiens
AAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 75 - DNA, 330 bp, Homo sapiens
  source 1..330: mol_type = genomic DNA
gcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 76 - DNA, 336 bp, Homo sapiens
  source 1..336: mol_type = genomic DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 77 - protein, 22 aa, synthetic construct
GSGATNFSLLKQAGDVEENPGP

SEQ ID NO: 78 - DNA, 66 bp, synthetic construct
  source 1..66: mol_type = other DNA
ggatctggagcaacaaacttctcactactcaaacaagcaggtgacgtggaggagaatcccggaccc

SEQ ID NO: 79 - protein, 21 aa, unidentified
MGYRMQLLSCIALSLALVTNS

SEQ ID NO: 80 - DNA, 63 bp, unidentified
  source 1..63: mol_type = other DNA
atgggttacaggatgcaactcctgtcttgcattgcactaagtcttgcacttgtcacaaacagt

SEQ ID NO: 81 - protein, 158 aa, Homo sapiens
GYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 82 - DNA, 474 bp, Homo sapiens
  source 1..474: mol_type = genomic DNA
ggctactttggcaagcttgaatctaaattatcagtcataagaaatttgaatgaccaagttctcttcattgaccaaggaaatcggcctctatttgaagatatgactgattctgactgtagagataatgcaccccggaccatatttattataagtatgtataaagatagccagcctagaggtatggctgtaactatctctgtgaagtgtgagaaaatttcaactctctcctgtgagaacaaaattatttcctttaaggaaatgaatcctcctgataacatcaaggatacaaaaagtgacatcatattctttcagagaagtgtcccaggacatgataataagatgcaatttgaatcttcatcatacgaaggatactttctagcttgtgaaaaagagagagacctttttaaactcattttgaaaaaagaggatgaattgggggatagatctataatgttcactgttcaaaacgaagac

SEQ ID NO: 83 - protein, 110 aa, synthetic construct
AAAIEVMYPPPYLDNEKSNGTIIHVKGKHLCPSPLFPGPSKPFWVLVVVGGVLACYSLLVTVAFIIFWVRSKRSRLLHSDYSNVTPRRPGPTRKHYQPYAPPRDFAAYRS

SEQ ID NO: 84 - DNA, 330 bp, synthetic construct
  source 1..330: mol_type = other DNA
gcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactactcaaatgttactccccgccgccccgggcccacccgcaagcattaccagccctatgccccaccacgcgacttcgcagcctatcgctcc

SEQ ID NO: 85 - protein, 6 aa, synthetic construct
HHHHHH

SEQ ID NO: 86 - protein, 4 aa, synthetic construct
GGSG